(19) United States
(12) Patent Application Publication
(10) Pub. No.: US 2013/0059850 A1
(43) Pub. Date: Mar. 7, 2013

(54) AZA-INDOLE DERIVATIVES USEFUL AS MODULATORS OF FAAH

(71) Applicants: Abbas M Walji, Lansdale, PA (US); Philippe G. Nantermet, Lansdale, PA (US); Keith P. Moore, Bensalem, PA (US); Rachel Anne Storr, Philadelphia, PA (US); Laura Vassallo, Drexel Hill, PA (US); Constantine Kreatsoulas, Elkins Park, PA (US)

(72) Inventors: Abbas M Walji, Lansdale, PA (US); Philippe G. Nantermet, Lansdale, PA (US); Keith P. Moore, Bensalem, PA (US); Rachel Anne Storr, Philadelphia, PA (US); Laura Vassallo, Drexel Hill, PA (US); Constantine Kreatsoulas, Elkins Park, PA (US)

(73) Assignee: MERCK SHARP & DOHME CORP., Rahway, NJ (US)

(21) Appl. No.: 13/696,520

(22) PCT Filed: May 4, 2011
(86) PCT No.: PCT/US2011/035091

§ 371 Date: Nov. 6, 2012

Related U.S. Application Data
(60) Provisional application No. 61/331,974, filed on May 6, 2010.

(51) Int. Cl. C07D 471/04; A61K 31/506; A61K 31/5377; A61P 25/16; A61P 29/00; A61P 25/06; A61P 25/00; A61P 25/28; A61K 31/437; A61P 19/02

(52) U.S. Cl. 514/234.5; 546/113; 544/316; 544/127; 514/300; 514/274

(57) ABSTRACT

The present invention is directed to certain Aza-Indole derivatives which are useful as modulators of Fatty Acid Amide Hydrolase (FAAH) and as FAAH imaging agents. The invention is also concerned with pharmaceutical formulations comprising these compounds as active ingredients and the use of the compounds and their formulations in the treatment of certain disorders, including osteoarthritis, rheumatoid arthritis, diabetic neuropathy, postherpetic neuralgia, skeletomuscular pain, and fibromyalgia, as well as acute pain, migraine, sleep disorder, Alzheimer Disease, and Parkinson's Disease.

### CROSS REFERENCE TO RELATED APPLICATION

This application claims priority from U.S. Provisional Application Ser. No. 61/331,974, filed May 6, 2010.

### BACKGROUND OF THE INVENTION

Disclosed herein are compounds that inhibit the activity of fatty acid amide hydrolase (FAAH), compositions that include the compounds, and methods of their use. Compounds disclosed herein as inhibitors of fatty acid amide hydrolase (FAAH) are useful in the treatment of diseases, disorders, or conditions that would benefit from the inhibition of fatty acid amide hydrolase and increases in endogenous fatty acid amides.

Fatty acid amide hydrolase (FAAH) is an enzyme that is abundantly expressed throughout the CNS (Freund et al. Physiol. Rev. 2003; 83:1017-1066) as well as in peripheral tissues, such as, for example, in the pancreas, brain, kidney, skeletal muscle, placenta, and liver (Giang, D. K. et al., Proc. Natl. Acad. Sci. U.S.A. 1997, 94, 2238-2242; Cravatt et al. Proc. Natl. Acad. Sci. U.S.A. 2004, 101, 29, 10821-10826). FAAH hydrolyzes the fatty acid amide (FAA) family of endogenous signaling lipids. General classes of fatty acid amides include the N-acylethanolamides (NAEs) and fatty acid primary amides (FAPAs). Examples of NAEs include anandamide (AEA), palmitoylethanolamide (PEA) and oleoylethanolamide (OEA). An example of FAPAs includes 9-Z-octadecenamide or oleamide. (McKinney M K and Cravatt B F 2005. Annu Rev Biochem 74:411-32). Another class of fatty acid amide family of endogenous signaling lipids is N-acyl taurines that have also been shown to be elevated upon FAAH deletion or inhibition and appear to act on transient receptor potential (TRP) family of calcium channels, although the functional consequences are not yet clear (Saghatelian A, et al. Biochemistry. 2004, 43:14332-9, Saghatelian A, et al. Biochemistry, 2006, 45:9007-9015). In addition to fatty acid amides, FAAH can also hydrolyze certain fatty acid esters, such as, for example, 2-arachidonylglycerol (2-AG) another endocannabinoid (Mechoulam et al. Biochem. Pharinacol. 1995; 50:83-90; Stella et al. Nature, 1997; 388:773-778; Suguria et al. Biochem. Biophys. Res. Commun. 1995; 215:89-97).

Inhibition of FAAH is expected to lead to an increase in the level of anandamide and other fatty acid amides. This increase in fatty acid amides leads to an increase in the nociceptive threshold. Thus, inhibitors of FAAH are useful in the treatment of pain (Cravatt, B F; Lichtman, A H Current Opinion in Chemical Biology 2003, 7, 469-475). Such inhibitors are useful in the treatment of other disorders that can be treated using fatty acid amides or modulators of cannabinoid receptors, such as, for example, anxiety, sleep disorder, Alzheimer disease, and Parkinson's disease, eating disorders, metabolic disorders, cardiovascular disorders, and inflammation (Simon et al Archives of Gen. Psychiatry, 2006, 63, 824-830. Kunos, G et al. Pharmacol Rev 2006, 58, 389-462). In some embodiments, FAAH inhibitor compounds may be peripherally restricted and may not substantially affect neural disorders, such as, for example, depression and anxiety. Finally, agonism of cannabinoid receptors has also been shown to reduce the progression of atherosclerosis in animal models (see Steffens et al. Nature, 2005, 434, 782-786; and Steffens et al., Curr Opin. Lipid., 2006, 17, 519-526). Thus, increasing the level of endogenous cannabinergic fatty acid amides (e.g., anandamide) is expected to effectively treat or reduce the risk of developing atherosclerosis.

Inhibition of FAAH also leads to elevation of palmitoylethanolamide which is thought to work, in part, through activation of the peroxisome proliferator-activated receptor a (PPAR-α) to regulate multiple pathways including, for example, pain perception in neuropathic and inflammatory conditions such as convulsions, neurotoxicity, spacticity and to reduce inflammation, for example, in atopic eczema and arthritis (LoVerme J et al. The nuclear receptor peroxisome proliferator-activated receptor-alpha mediates the anti-inflammatory actions of palmitoylethanolamide. Mol Pharmacol 2005, 67, 15-19; LoVerme J et al. The search for the palmitoylethanolamide receptor. Life Sci 2005, 77: 1685-1698. Lambert D M et al. The palmitoylethanolamide family: a new class of anti-inflammatory agents? Curr Med Chem 2002, 9: 663-674; Eberlein B, et al. Adjuvant treatment of atopic eczema: assessment of an emollient containing N-palmitoylethanolamine (ATOPA study). J Eur Acad Dermatol Venereol. 2008, 22:73-82. Re G, et al. Palmitoylethanolamide, endocannabinoids and related cannabimimetic compounds in protection against tissue inflammation and pain: potential use in companion animals. Vet J. 2007 173:21-30.). Thus, inhibition of FAAH is useful for the treatment of various pain and inflammatory conditions, such as osteoarthritis, rheumatoid arthritis, diabetic neuropathy, postherpetic neuralgia, skeletomuscular pain, and fibromyalgia.

It is also thought that certain fatty acid amides, such as, for example, OEA, act through the peroxisome proliferator-activated receptor a (PPAR-α) to regulate diverse physiological processes, including, e.g., feeding and lipolysis. Consistent with this, human adipose tissue has been shown to bind and metabolize endocannabinoids such as anandamide and 2-arachidonylglycerol (see Spoto et al., Biochimie 2006, 88, 1889-1897; and Matias et al., J. Clin. Endocrin. & Met., 2006, 91, 3171-3180). Thus, inhibiting FAAH activity in vivo leads to reduced body fat, body weight, caloric intake, and liver triglyceride levels. However, unlike other anti-lipidemic agents that act through PPAR-α, e.g., fibrates, FAAH inhibitors do not cause adverse side effects such as rash, fatigue, headache, erectile dysfunction, and, more rarely, anemia, leukopenia, angioedema, and hepatitis (see, e.g., Muscari, et al., Cardiology, 2002, 97:115-121).

Many fatty acid amides are produced on demand and rapidly degraded by FAAH. As a result, hydrolysis by FAAH is considered to be one of the essential steps in the regulation of fatty acid amide levels in the central nervous system as well as in peripheral tissues and fluids. The broad distribution of FAAH combined with the broad array of biological effects of fatty acid amides (both endocannabinoid and non-endocannabinoid mechanisms) suggests that inhibition of FAAH leads to altered levels of fatty acid amides in many tissues and fluids and may be useful to treat many different conditions. FAAH inhibitors increase the levels of endogenous fatty acid amides. FAAH inhibitors block the degradation of endocannabinoids and increase the tissue levels of these endogenous substances. FAAH inhibitors can be used in this respect in the prevention and treatment of pathologies in which endogenous cannabinoids and or any other substrates metabolized by the FAAH enzyme are involved.

The various fatty acid ethanolamides have important and diverse physiological functions. As a result, inhibitor molecules that selectively inhibit FAAH enzymatic activity would allow a corresponding selective modulation of the cellular and extra-cellular concentrations of a FAAH substrate. FAAH inhibitors that are biologically compatible could be effective pharmaceutical compounds when formulated as therapeutic agents for any clinical indication where FAAH enzymatic inhibition is desired. In some embodiments, FAAH activity in peripheral tissues can be preferentially inhibited. In some embodiments, FAAH inhibitors that do substantially cross the blood-brain-barrier can be used to preferentially inhibit FAAH activity in peripheral tissues. In some embodiments, FAAH inhibitors that preferentially inhibit FAAH activity in peripheral tissues can minimize the effects of FAAH inhibition in the central nervous system. In some embodiments, it is preferred to inhibit FAAH activity in peripheral tissues and minimize FAAH inhibition in the central nervous system.

### SUMMARY OF THE INVENTION

The present invention is directed to certain Aza-Indole derivatives which are useful as inhibitors of Fatty Acid Amide Hydrolase (FAAH). The invention is also concerned with pharmaceutical formulations comprising these compounds as active ingredients and the use of the compounds and their formulations in the treatment of certain disorders, including osteoarthritis, rheumatoid arthritis, diabetic neuropathy, postherpetic neuralgia, skeletomuscular pain, and fibromyalgia, as well as acute pain, migraine, sleep disorder, Alzheimer disease, and Parkinson's disease.

### DETAILED DESCRIPTION OF THE INVENTION

In one aspect the invention is directed to a compound of the formula I:

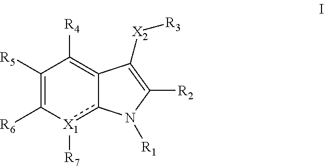

or a pharmaceutically acceptable salt thereof wherein:
n is 0, 1 or 2;
X₁ is selected from C or N;

### X₂ is S or SO or SO₂;

R₁ is selected from the group consisting of
(1) hydrogen,
(2) C₁₋₄alkyl,
(3) aryl,
(4) HET₁,
(5) (CH₂)-aryl, and
(6) (CH₂)-HET₁,
   wherein choice (2), and the aryl or HET₁ of choices (3), (4), (5) and (6) are optionally mono or di-substituted with substituents selected from hydroxyl, halo, CF₃ and OCH₃;
   R₂ is selected from the group consisting of:
(1) hydrogen,
(2) aryl,
(3) HET₂,
(4) (CH₂)-aryl,
(5) (CH₂)-HET₂,
(6) —C₁₋₆alkyl,
(7) —C₂₋₆alkenyl,
(8) —C₃₋₆cycloalkyl,
(9) —CH₂—C₃₋₆cycloalkyl,
(10) —C₃₋₆cycloalkenyl,
(11) —NH—(CH₂)-aryl,
(12) —CH₂—NH—R₁₉R₂₀,
(13) —NH—C₃₋₇cycloalkyl,
(14) —NH—C(O)R₈,
   wherein R₈ is selected from the group consisting of
(a) aryl,
(b) HET₃,
(c) (CH₂)-aryl,
(d) (CH₂)-HET₃,
(e) —C₁₋₆alkyl, and
(f) —C₃₋₇cycloalkyl,
(15) —C(O)NR₉R₁₀,
   wherein R₉ and R₁₀ are each independently selected from the group consisting of
(a) hydrogen,
(b) hydroxyl,
(c) aryl,
(d) HET₄,
(e) —C₃₋₆cycloalkyl, optionally substituted with 1 to 4 methyl groups,
(f) —OC₃₋₆cycloalkyl,
(g) —C₁₋₄alkyl, optionally mono or di-substituted with hydroxyl, HET₅, or C₃₋₆cycloalkyl,
(h) —OC₁₋₄alkyl,
(i) —C(O)CH₃,
(j) mono, di or tri-halo C₁₋₄alkyl, and
(k) mono, di or tri-halo —OC₁₋₄alkyl,
or
R₉ and R₁₀ are joined together to form a ring with the atoms to which they are attached there is formed a heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl, —S(O)nC₁₋₄alkyl, and C(O)—NRaRb, wherein Ra and Rb are each independently selected from hydrogen and methyl,
   wherein R₂ choices (2), (3), (4), (5), (6), (7), (8), (9), (10), (11) and (13) are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
   wherein R₁₁, R₁₂, R₁₃, R₁₄, R₁₅, R₁₆, R₁₇, R₁₈, R₁₉, are each independently selected from H and C₁₋₄alkyl, optionally substituted with hydroxyl, and
   R₂₀ is selected from H and C₁₋₄alkyl optionally substituted with aryl, HET₆, optionally substituted with hydroxyl or 1-4 methyl groups,
   or R₁₁ and R₁₂ or R₁₃ and R₁₄ or R₁₉ and R₂₀ can be joined together to form a ring with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, halo C₁₋₄alkyl, —C(O)—C₁₋₄alkyl and —S(O)ₙC₁₋₄alkyl;
   R₃ is selected from the group consisting of:
(1) aryl,
(2) HET₇,
(3) —C₁₋₆alkyl,
(4) —C₃₋₆cycloalkyl, and
(5) mono, di or tri-halo C₃₋₆cycloalkyl,
   wherein choices (1), (2) and (3) are each optionally mono or di-substituted with substituents independently selected from the group consisting of
(a) hydroxy,
(b) halo,
(c) —CF₃,
(d) —OCF₃,
(e) methyl, and
(f) methoxy;
   R₄, R₅ and R₆ are each independently selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₆cycloalkyl;
   wherein choice (7), and the aryl or HET₅ of choices (3), (4), (5) and (6) are optionally mono or di-substituted with substituents selected from hydroxyl, halo, CF₃ and OCH₃;
   R₇ is selected from the group consisting of:
(1) hydrogen,
(2) halogen,
(3) HET₈, and
(4) —C₁₋₆alkyl,
   wherein choices (3) and (4) are each optionally mono or di-substituted with substituents selected from hydroxyl, C₃₋₆cycloalkyl, —C(O)—NH₂, phenyl and HET₉, with the proviso that R₇ is other than halogen when X₁ is N.

Within this aspect there is a genus wherein:
X₁ is N.

Within this aspect there is a genus wherein:
X₂ is S.

Within this aspect there is a genus wherein:

R₁ is selected from the group consisting of:
(1) hydrogen, and
(2) C₁₋₄alkyl,
   wherein choice (2), is optionally mono or di-substituted with substituents selected from hydroxyl, halo, CF₃ and OCH₃.

Within this aspect there is a genus wherein:

R₂ is selected from the group consisting of:
(1) hydrogen,
(2) aryl,
(3) (CH₂)-aryl,
(4) (CH₂)-HET₂,
(5) —C₁₋₆alkyl,
(6) —C₃₋₆cycloalkyl,
(7) —CH₂—C₃₋₆cycloalkyl,
(8) —C₃₋₆cycloalkenyl,
(9) —CH₂—NH—R₁₉R₂₀,
(10) —NH—C₃₋₆cycloalkyl, and
(11) —C(O)NR₉R₁₀,
   wherein R₉ and R₁₀ are each independently selected from the group consisting of
(a) hydrogen,
(c) aryl,
(d) HET₄,
(e) —C₃₋₆cycloalkyl, optionally substituted with 1 to 4 methyl groups,
(f) —OC₃₋₆cycloalkyl,
(g) —C₁₋₄alkyl, optionally mono or di-substituted with hydroxyl, HET₅, or C₃₋₆cycloalkyl, and
(h) —OC₁₋₄alkyl,
or
R₉ and R₁₀ are joined together to form a ring with the atoms to which they are attached there is formed a heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl, —S(O)ₙC₁₋₄alkyl, and C(O)—NRaRb, wherein Ra and Rb are each independently selected from hydrogen and methyl,
   wherein R₂ choices (2), (3), (4), (5), (6), (7), (8), (9) and (10) are each optionally mono or di-substituted with substituents independently selected from the group consisting of
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
   wherein R₁₁, R₁₂, R₁₃, R₁₄, R₁₅, R₁₆, R₁₇, R₁₈, R₁₉, are each independently selected from H and C₁₋₄alkyl, optionally substituted with hydroxyl, and
   R₂₀ is selected from H and C₁₋₄alkyl optionally substituted with aryl, HET₆, optionally substituted with hydroxyl or 1-4 methyl groups, or
   R₁₁ and R₁₂ or R₁₃ and R₁₄ or R₁₉ and R₂₀ can be joined together to form a ring with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, —C(O)—C₁₋₄alkyl and —S(O)ₙC₁₋₄alkyl.

Within this genus there is a sub-genus wherein

R₂ is selected from the group consisting of:
(1) phenyl,
(2) —C₁₋₆alkyl, and
(3) —C(O)NR₉R₁₀,
   wherein R₉ and R₁₀ are each independently selected from the group consisting of
   (a) aryl,
   (b) HET₄,
   (c) —C₃₋₆cycloalkyl, optionally substituted with 1 to 4 methyl groups,
   (d) —C₁₋₄alkyl, optionally mono or di-substituted with hydroxyl, HET₅, or C₃₋₆cycloalkyl,
   or
   R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
      wherein R₂ choices (1), (2) and (3), are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF_{3>}—NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
   wherein R₁₁, R₁₂, R₁₃, R₁₄, R₁₅, R₁₆, R₁₇, R₁₈, are each independently selected from H and C₁₋₄alkyl, optionally substituted with hydroxyl.

Within this genus there is sub-genus wherein:

R₂ is selected from the group consisting of:
(1) phenyl,
(2) —C₁₋₆alkyl, and
(3) —C(O)NR₉R₁₀,
   wherein R₉ and R₁₀ are each independently selected from the group consisting of
   (a) aryl,
   (b) HET₄,
   (c) —C₃₋₆cycloalkyl, optionally substituted with 1 to 4 methyl groups,
   (d) optionally mono or di-substituted with hydroxyl, HET₅, or C₃₋₆cycloalkyl,
   or
   R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
      wherein R₂ choices (1), (2) and (3), are each optionally mono or di-substituted with substituents independently selected from the group consisting of:
(a) halo,
(b) mono, di or tri-halo C₁₋₄ alkyl,
(c) —OC₁₋₄ alkyl, optionally substituted with hydroxyl, halo or amino,
(d) —C₁₋₄alkyl optionally substituted with one or two substituents selected from hydroxyl, CN, —CHF₂, —CF₃, —NH₂, and —OCH₃,
(e) —C(O)—O-aryl,
(f) —C(O)—NR₁₃R₁₄,
(g) —NR₁₇R₁₈, and
(h) hydroxyl,
   wherein R₁₃, R₁₄, R₁₇, R₁₈, are each independently selected from H and C₁₋₄allyl, optionally substituted with hydroxyl.

Within this aspect there is a genus wherein:

R₃ is selected from the group consisting of:
(1) aryl, and
(2) HET₇,
   wherein choices (1) and (2) are each optionally mono or di-substituted with substituents independently selected from the group consisting of:
(a) halo, and
(b) methyl.

Within this genus there is a sub-genus wherein:

R₃ is an optionally substituted:
(1) phenyl,
(2) pyridyl,
(3) pyridazinyl, and
(4) pyrimidyl.

Within this aspect there is a genus wherein:

R₄ and R₅ are each hydrogen.

Within this aspect there is a genus wherein:

R₇ is selected from the group consisting of:
(1) hydrogen,
(2) halogen, and
(3) HET₈,
   wherein choice (3) is optionally mono or di-substituted with substituents selected from hydroxyl, C₃₋₆cycloalkyl, —C(O)—NH₂, phenyl and HET₉.

Within this aspect there is a genus of compound of the formula

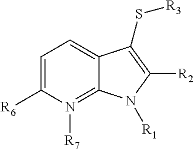

or a pharmaceutically acceptable salt thereof wherein
n is 0, 1 or 2;
R₁ is selected from the group consisting of:
(1) hydrogen, and
(2) C₁₋₄ alkyl,
   wherein choice (2), is optionally mono or di-substituted with substituents selected from hydroxyl, halo, CF₃ and OCH₃;
   R₂ is selected from the group consisting of:
(1) phenyl,
(2) —C₁₋₆alkyl, and
(3) —C(O)NR₉R₁₀,
   wherein R₉ and R₁₀ are each independently selected from the group consisting of
   (a) aryl,
   (b) HET₄,
   (c) —C₃₋₆cycloalkyl, optionally substituted with 1 to 4 methyl groups,
   (d) —C₁₋₄alkyl, optionally mono or di-substituted with hydroxyl, HET₅, or C₃₋₆cycloalkyl,
   or
   R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
      wherein R₂ choices (1), (2) and (3), are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
   wherein R₆, R₇, R₈, R₉, R₁₀, R₁₁, R₁₂, R₁₃, R₁₄, R₁₅, R₁₆, R₁₇, R₁₈, are each independently selected from H and C₁₋₄allyl, optionally substituted with hydroxyl;
   R₃ is selected from the group consisting of
(1) aryl, and
(2) HET₇,
   wherein choices (1) and (2) are each optionally mono or di-substituted with substituents independently selected from the group consisting of:
(a) halo, and
(b) methyl;
   R₆ is selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₇cycloalkyl;
   wherein choice (7), and the aryl or HET₅ of choices (3), (4), (5) and (6) are optionally mono or di-substituted with substituents selected from hydroxyl, halo, CF₃ and OCH₃; and
   R₇ is selected from the group consisting of:
(1) hydrogen,
(2) halogen, and
(3) HET₈,
   wherein choice (3) is optionally mono or di-substituted with substituents selected from hydroxyl, C₃₋₆cycloalkyl, —C(O)—NH₂, phenyl and HET₉.

Within this genus there is a sub-genus of compound of formula I

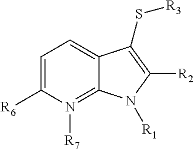

or a pharmaceutically acceptable salt thereof wherein:
n is 0, 1 ort;
R₁ is selected from the group consisting of:
(1) hydrogen, and
(2) C₁₋₄alkyl,
   wherein choice (2), is optionally mono or di-substituted with substituents selected from hydroxyl, halo, CF₃ and OCH₃;
   R₂ is selected from the group consisting of:
(1) phenyl,
(2) —C₁₋₆alkyl,
(3) —C(O)NR₉R₁₀,
   wherein R₉ and R₁₀ are each independently selected from the group consisting of (a) aryl,
   (b) HET₄,
   (c) —C₃₋₆cycloalkyl, optionally substituted with 1 to 4 methyl groups,
   (d) —C₁₋₄alkyl, optionally mono or di-substituted with hydroxyl, HET₅, or C₃₋₆cycloalkyl,
   or
   R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
      wherein R₂ choices (1), (2) and (3), are each optionally mono or di-substituted with substituents independently selected from the group consisting of (a) halo,
(b) mono, di or tri-halo C₁₋₄ alkyl,
(c) —OC₁₋₄ alkyl, optionally substituted with hydroxyl, halo or amino,
(d) —C₁₋₄alkyl optionally substituted with one or two substituents selected from hydroxyl, CN, —CF₃, —NH₂, and —OCH₃,
(e) —C(O)—O-aryl,
(f) —C(O)—NR₁₃R₁₄,
(g) —NR₁₇R₁₈, and
(h) hydroxyl,
   wherein R₁₃, R₁₄, R₁₇, R₁₈, are each independently selected from H and C₁₋₄alkyl, optionally substituted with hydroxyl.
   R₃ is selected from
(1) phenyl,
(2) pyridyl,
(3) pyridazinyl, and
(4) pyrimidyl,
   wherein R₃ is optionally mono or di substituted with substituents selected from the group consisting of halo and methyl.
   R₆ is selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₇cycloalkyl;
   wherein choice (7), and the aryl or HET₅ of choices (3), (4), (5) and (6) are optionally mono or di-substituted with substituents selected from hydroxyl, halo, CF₃ and OCH₃; and
   R₇ is selected from the group consisting of:
(1) hydrogen,
(2) halogen, and
(3) HET₈,
   wherein choice (3) is optionally mono or di-substituted with substituents selected from hydroxyl, C₃₋₆cycloalkyl, —C(O)—NH₂, phenyl and HET₉.

In another aspect, the invention is directed to pharmaceutical compositions which comprise an inert carrier and a compound of Formula Ior a pharmaceutically acceptable salt thereof.

In another aspect, the invention is directed to a method of treating a FAAH mediated disease in a patient in need of such treatment comprising: administration to a patient in need of such treatment of a therapeutically effective amount of a compound of formula I, according to claim 1 and a pharmaceutically acceptable carrier.

In another aspect, the invention is directed to a method of treating a disease is selected from osteoarthritis, rheumatoid arthritis, diabetic neuropathy, postherpetic neuralgia, pain, fibromyalgia, pain, migraine, sleep disorder, Alzheimer Disease, and Parkinson's Disease comprising: administration to a patient in need of such treatment of a therapeutically effective amount of a compound of formula I, and a pharmaceutically acceptable carrier.

In another aspect the invention is directed to the use of a compound according of Formula I or a pharmaceutically acceptable salt thereof for the manufacture of a medicament for the treatment of a physiological disorder associated with an excess of FAAH in a mammal.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ²³I and ¹²⁵I, and chlorine such as ³⁶Cl.

Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The invention is described using the following definitions unless otherwise indicated.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkylthio” means alkylthio groups having the indicated number of carbon atoms of a straight, branched or cyclic configuration. C₁₋₆alkylthio, for example, includes methylthio, propylthio, isopropylthio, and the like.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes propynyl, 1-methylethynyl, butyryl and the like.

The term “cycloalkyl” means mono-, bi- or tricyclic structures, optionally combined with linear or branched structures, the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, and the like.

The term “aryl” is defined as a mono- or bi-cyclic aromatic ring system and includes, for example, phenyl, naphthyl, and the like.

The term “aralkyl” means an alkyl group as defined above of 1 to 6 carbon atoms with an aryl group as defined above substituted for one of the alkyl hydrogen atoms, for example, benzyl and the like.

The term “aryloxy” means an aryl group as defined above attached to a molecule by an oxygen atom (aryl-O) and includes, for example, phenoxy, naphthoxy and the like.

The term “aralkoxy” means an aralkyl group as defined above attached to a molecule by an oxygen atom (aralkyl-O) and includes, for example, benzyloxy, and the like.

The term “arylthio” is defined as an aryl group as defined above attached to a molecule by a sulfur atom (aryl-5) and includes, for example, thiophenyoxy, thionaphthoxy and the like.

The term “aroyl” means an aryl group as defined above attached to a molecule by an carbonyl group (aryl-C(O)—) and includes, for example, benzoyl, naphthoyl and the like.

The term “aroyloxy” means an aroyl group as defined above attached to a molecule by an oxygen atom (aroyl-O) and includes, for example, benzoyloxy or benzoxy, naphthoyloxy and the like.

The term “HET”, such as in “HET₁”, “HET₂”, “HET₃”, “HET⁴”, “HET₅”, “HET₆”, “HET₇”, “HET₈” or “HET₉” is defined as a 5- to 10-membered aromatic, partially aromatic or non-aromatic mono- or bicyclic ring, containing 1-4 heteroatoms selected from O, S and N, and optionally substituted with 1-2 oxo groups. Where applicable, the Het group shall be defined to include the N-oxide. Preferably, “HET” is a 5- or 6-membered aromatic or non-aromatic monocyclic ring containing 1-3 heteroatoms selected from O, S and N, for example, pyridine, pyrimidine, pyridazine, furan, thiophene, thiazole, oxazole, isooxazole and the like, or HET is a 9- or 10-membered aromatic or partially aromatic bicyclic ring containing 1-3 heteroatoms selected from O, S, and N, for example, benzofuran, benzothiophene, indole, pyranopyrrole, benzopyran, quionoline, benzocyclohexyl, naphtyridine and the like. “HET” also includes the following: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl. In one aspect “HET” is selected from pyridyl, pyridazinyl, pyrimidinyl, pyrazinyl, thiazolyl, thienyl, pyrrolyl, oxazolyl, and oxadiazole;

For all of the above definitions, each reference to a group is independent of all other references to the same group when referred to in the Specification. For example, if both R¹ and R² are HET, the definitions of HET are independent of each other and R¹ and R² may be different HET groups, for example furan and thiophene.

The ability of the compounds of Formula Ito selectively inhibit FAAH makes them useful for treating, preventing or reversing the progression of a variety of inflammatory and non-inflammatory diseases and conditions.

Diseases, disorders, syndromes and/or conditions, that would benefit from inhibition of FAAH enzymatic activity include, for example, Alzheimer's Disease, schizophrenia, depression, alcoholism, addiction, suicide, Parkinson's disease, Huntington's disease, stroke, emesis, miscarriage, embryo implantation, endotoxic shock, liver cirrhosis, atherosclerosis, cancer, traumatic head injury, glaucoma, and bone cement implantation syndrome.

Other diseases, disorders, syndromes and/or conditions that would benefit from inhibition of FAAH activity, include, for example, multiple sclerosis, retinitis, amyotrophic lateral sclerosis, immunodeficiency virus-induced encephalitis, attention-deficit hyperactivity disorder, pain, nociceptive pain, neuropathic pain, inflammatory pain, noninflammatory pain, painful hemorrhagic cystitis, obesity, hyperlipidemia, metabolic disorders, feeding and fasting, alteration of appetite, stress, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, and inflammatory diseases. Neurological and psychological disorders that would benefit from inhibition of FAAH activity include, for example, pain, depression, anxiety, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, diskenesia, seizure disorders, jet lag, and insomnia.

FAAH inhibitors can also be used in the treatment of a variety of metabolic syndromes, diseases, disorders and/or conditions, including but not limited to, insulin resistance syndrome, diabetes, hyperlipidemia, fatty liver disease, obesity, atherosclerosis and arteriosclerosis. FAAH inhibitors are useful in the treatment of a variety of painful syndromes, diseases, disorders and/or conditions, including but not limited to those characterized by non-inflammatory pain, inflammatory pain, peripheral neuropathic pain, central pain, deafferentiation pain, chronic nociceptive pain, stimulus of nociceptive receptors, phantom and transient acute pain.

Inhibition of FAAH activity can also be used in the treatment of a variety of conditions involving inflammation. These conditions include, but are not limited to arthritis (such as rheumatoid arthritis, shoulder tendonitis or bursitis, gouty arthritis, and aolymyalgia rheumatica), organ-specific inflammatory diseases (such as thyroiditis, hepatitis, inflammatory bowel diseases), asthma, other autoimmune diseases (such as multiple sclerosis), chronic obstructive pulmonary disease (COPD), allergic rhinitis, and cardiovascular diseases.

In some cases, FAAH inhibitors are useful in preventing neurodegeneration or for neuroprotection.

In addition, it has been shown that when FAAH activity is reduced or absent, one of its substrates, anandamide, acts as a substrate for COX-2, which converts anandamide to prostamides (Weber et al J Lipid. Res. 2004; 45:757). Concentrations of certain prostamides may be elevated in the presence of a FAAH inhibitor. Certain prostamides are associated with reduced intraocular pressure and ocular hypotensivity. Thus, in one embodiment, FAAH inhibitors may be useful for treating glaucoma.

In some embodiments, FAAH inhibitors can be used to treat or reduce the risk of EMDs, which include, but are not limited to, obesity, appetite disorders, overweight, cellulite, Type I and Type II diabetes, hyperglycemia, dyslipidemia, steatohepatitis; liver steatosis, non-alcoholic steatohepatitis, Syndrome X, insulin resistance, diabetic dyslipidemia, anorexia, bulimia, anorexia nervosa, hyperlipidemia, hypertriglyceridemia, atherosclerosis, arteriosclerosis, inflammatory disorders or conditions, Alzheimer's disease, Crohn's disease, vascular inflammation, inflammatory bowel disorders, rheumatoid arthritis, asthma, thrombosis, or cachexia.

In other embodiments, FAAH inhibitors can be used to treat or reduce the risk of insulin resistance syndrome and diabetes, i.e., both primary essential diabetes such as Type I Diabetes or Type II Diabetes and secondary nonessential diabetes. Administering a composition containing a therapeutically effective amount of an in vivo FAAH inhibitor reduces the severity of a symptom of diabetes or the risk of developing a symptom of diabetes, such as atherosclerosis, hypertension, hyperlipidemia, liver steatosis, nephropathy, neuropathy, retinopathy, foot ulceration, or cataracts.

In another embodiment, FAAH inhibitors can be used to treat food abuse behaviors, especially those liable to cause excess weight, e.g., bulimia, appetite for sugars or fats, and non-insulin-dependent diabetes.

In some embodiments, FAAH inhibitors can be used to treat a subject suffering from an EMD and also suffers from a depressive disorder or from an anxiety disorder. Preferably, the subject is diagnosed as suffering from the depressive or psychiatric disorder prior to administration of the FAAH inhibitor composition. Thus, a dose of a FAAH inhibitor that is therapeutically effective for both the EMD and the depressive or anxiety disorder is administered to the subject.

Preferably, the subject to be treated is human. However, the methods can also be used to treat non-human mammals. Animal models of EMDs such as those described in, e.g., U.S. Pat. No. 6,946,491 are particularly useful.

FAAH inhibitor compositions can also be used to decrease body-weight in individuals wishing to decrease their body weight for cosmetic, but not necessarily medical considerations.

A FAAH inhibitor composition can be administered in combination with a drug for lowering circulating cholesterol levels (e.g., statins, niacin, fibric acid derivatives, or bile acid binding resins). FAAH inhibitor compositions can also be used in combination with a weight loss drug, e.g., orlistat or an appetite suppressant such as diethylpropion, mazindole, orlistat, phendimetrazine, phentermine, or sibutramine.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

The following abbreviations have the indicated meanings:
AIBN=2,2′-azobisisobutyronitrile
B.P.=benzoyl peroxide
Bn=benzyl
CCl₄=carbon tetrachloride
D=—O(CH₂)₃O—
DAST=diethylamine sulfur trifluoride
DCC=dicyclohexyl carbodiimide
DCI=1-(3-dimethylaminopropyl)-3-ethyl carbodiimide
DEAD=diethyl azodicarboxylate
DIBAL=diisobutyl aluminum hydride
DME=ethylene glycol dimethylether
DMAP=4-(dimethylamino)pyridine
DMF=N,N-dimethylformamide
DMSO=dimethyl sulfoxide
Et₃N=triethylamine
HRMS=high resolution mass spectrometry
LCMS=liquid chromatography mass spectrometry
LDA=lithium diisopropylamide
m-CPBA=metachloroperbenzoie acid
NBS=N-bromosuccinimide
NSAID=non-steroidal anti-inflammatory drug
PCC=pyridiniurn chlorochromate
PDC=pyridinium dichromate
Ph=phenyl
1,2-Ph=1,2-benzenediyi
Pyr=pyridinediyl
Qn=7-chloroquinolin-2-yl

### Rₛ=—CH₂SCH₂CH₂Ph

r.t.=room temperature
rac.=racemic
THF=tetrahydrofuran
THP=tetrahydropyran-2-yl

### Alkyl Group Abbreviations

Me=methyl
Et=ethyl
n-Pr=normal propyl
i-Pr=isopropyl
n-Bu=normal butyl
i-Bu=isobutyl
s-Bu=secondary butyl
t-Bu=tertiary butyl
c-Pr=cyclopropyl
c-Bu=cyclobutyl
c-Pen=cyclopentyl
c-Hex=cyclohexyl

Some of the compounds described herein contain one or more asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that in the discussion of methods of treatment which follows, references to the compounds of Formula I are meant to also include the pharmaceutically acceptable salts.

The magnitude of prophylactic or therapeutic dose of a compound of Formula I will, of course, vary with the nature and the severity of the condition to be treated and with the particular compound of Formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for oral administration to humans may contain from about 0.5 mg to about 5 g of active agent compounded with an appropriate and convenient amount of carrier material which may vary from about 5 to about 95 percent of the total composition. Dosage unit forms will generally contain from about 1 mg to about 2 g of an active ingredient, typically 25 mg, 50 mg, 100 mg, 200 mg, 300 mg, 400 mg, 500 mg, 600 mg, 800 mg, or 1000 mg.

For the treatment of FAAH mediated diseases the compound of Formula I may be administered orally, topically, parenterally, by inhalation spray or rectally in dosage unit formulations containing conventional non-toxic pharmaceutically acceptable carriers, adjuvants and vehicles. The term parenteral as used herein includes subcutaneous, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, solutions, aqueous or oily suspensions, dispersible powders or granules, emulsions, hard or soft capsules, syrups or elixirs, Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane dial. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing a compound of Formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

### Assays

The following assays illustrate the utility of the invention:

The compounds of the invention underwent pharmacological evaluations to determine their inhibitory effect on the enzyme FAAH (Fatty Acid Amide Hydrolase).

To assist in assay development stable cell lines for human, murine and rat full length FAAH were developed. Human FAAH cDNA (Accession No: NM_{—}001441.1) was purchased from Origene (Rockville, Md.). The full length FAAH was subcloned into the mammalian expression vector, pcDEF.neo, using XbaI and EcoRI restriction sites and used for stable cell line generation.

| Construct | Primer | Sequence |
|---|---|---|
| Full length rodent FAAH | 1 | CAAGGTACCGCCACCATGGTGCTGAGCGAAGTGTGG |
| Full length murine FAAH | 2 | CCGGAATTCTCAAGATGGCCGCTTTTCAGG |
| Full length rat FAAH | 3 | CCGGAATTCTCACGATGGCTGCTTTTGAGG |

Murine (accession number NM_{—}010173) and Rat FAAH (accession number NM_{—}024132) was amplified by reverse transcriptase polymerase chain reaction (RT-PCR) from brain cDNA (BD Biosciences, San Jose, Calif.) using primers 1 and 2 or primers 1 and 3 respectively (see Table). The resulting PCR product was ligated into pCR4 TOPO and DNA sequence confirmed. The full length murine FAAH was subcloned into the mammalian expression vector, pcDEFneo using either EcoRI (murine) or KpnI and EcoRI (rat) restriction sites. Chinese hamster ovary cells (CHO) were transfected following manufacturers protocol (AMAXA). Forty eight hours post transfection, cells were trypsinized and transferred to 96 well plates in Iscove's DMEM media supplemented with 2 mM Glutamine, 10% fetal calf serum, 1 mg/ml geneticin and HT Supplement (0.1 mM sodium hypoxanthine, 0.016 mM thymidine) in order to isolate single clones. Following selection in geneticin, individual clones were selected and FAAH activity was assessed using a whole cell fluorescent anandamide assay, modified from Ramarao et al (2005). Following removal of tissue culture media cells were dislodged following addition of Cellstripper (Mediatech, Inc. Manassas, Va.) and transferred to 96 well black clear bottom assay plate, centrifuged at 1,000 rpm for 3 mins and media removed and replaced with assay buffer (50 mM Tris pH8.0, 1 mM EDTA, 0.1% fatty acid free BSA). The reaction was initiated by addition of fluorescent substrate, AMC Arachidonoyl Amide (Cayman Chemical, Ann Arbor, Mich.) to 1 μM and reaction allowed to proceed for 2 hours at room temperature. Release of fluorescence was monitored in a CytoFluor Multiplate Reader. Cells expressing the highest amount of FAAH activity were selected for study with FAAH inhibitors.

### Preparation of Lysate and Microsomes

CHO cells expressing FAAH were used to prepare either crude cell lysate or microsome fractions. To harvest cells, tissue culture media was decanted, the monolayer washed three times with Ca⁺⁺Mg⁺⁺ free PBS and cells recovered after 15 mM in enzyme free dissociation media (Millipore Corp, Billerica, Mass.). Cells were collected by centrifuging at 2000 rpm for 15 min. and the cell pellet re-suspended with 50 mM HEPES (pH 7.4) containing 1 mM EDTA and the protease inhibitors aprotinin (1 mg/ml) and leupeptin (100 μM). The suspension was sonicated at 4° C. and the cell lysate recovered after centrifuging at 12,000×g (14,600 rpm, SS34 rotor) for 20 min at 4° C. to form a crude pellet of cell debris, nuclei, peroxisomes, lysosomes, and mitochondria; the supernatant or cell lysate was used for FAAH enzyme assay. In some cases, microsomes fractions enriched in FAAH were prepared by centrifuging the cell lysate further at 27,000 rpm (100,000×g) in SW28 rotor for 50 minutes at 4° C. The pellet containing FAAH-enriched microsomes was re-suspend in 50 mM HEPES, (pH 7.4) 1 mM EDTA, and any remaining DNA sheared by passage of material through a 23 gauge needle and aliquots of enzyme were store at −80° C. prior to use.

### FAAH Assays

Several assays have been used to demonstrate the inhibitory activity. Enzyme activity was demonstrated in a radioenzymatic test based on measuring the product of hydrolysis (ethanolamine [³H]) of anandamide [ethanolamine 1-.sup.3H] (American Radiolabeled Chemicals; 1 mCi/ml) with FAAH (Life Sciences (1995), 56, 1999-2005 and Journal of Pharmacology and Experimented Therapeutics (1997), 283, 729-734), Analytical. Biochemistry (2003), 318, 270-5. In addition, routine assays were performed monitoring hydrolysis of arachidonyl-7-amino-4-methylcoumarin amide (AAMCA) by following increase in fluorescence upon release of 7-amino 4-methyl coumarin (λ_{EX}=355 nm, μ_{EM}=460 nm). Analytical. Biochemistry (2005). 343, 143-51

Assays are performed on either cell lysate or microsome fractions prepared as described or in whole cell format employing either the fluorescent substrate AAMCA (Cayman chemical, Ann Arbor, Mich.,) or ³H-anandamide ([ETHANOLAMINE-1-3H] American Radiolabeled Chemicals; 1 mCi/ml). The cell lysate or microsome assay is performed in black PerkinElmer OptiPlates-384F by adding FAAH_CHO (whole cell (human whole cell or human WC), cell lysate (human cell lysate or human LY) or microsome) in assay buffer (50 mM Phosphate, pH 8.0, 1 mM EDTA, 200 mM KCl, 0.2% glycerol, 0.1% fatty acid free BSA) to each well, followed by either DMSO or compound and allowed to incubate at 22-25° C. for fifteen minutes. AAMCA substrate was used to achieve a final concentration of 1 μM and reaction allowed to proceed at room temperature for 1-3 hours. Fluorescent release as a measure of FAAH activity was monitored by reading the plate in a Envision plate Reader (Ex: 360/40 nM; Em: 460/40 nM). Whole cell assay is conducted with cells harvested after rinsing tissue culture flasks three times with Ca⁺⁺Mg⁺⁺ free PBS, incubating for 10 mM in Enzyme free dissociation media and centrifuging for 5 minutes at 1,000 rpm in table top centrifuge. Cells are resuspended in assay buffer at desired cell number in (4×10⁴ cells/assay in 96-well format; 1×10⁴ cells/assay in 384-well format) and assayed as described.

Alternatively, assays are performed using anandamide [ethanolamine 1-.sup.3H] (specific activity of 10 Ci/mmol) diluted with cold anandamide to achieve a final assay concentration of 1 μM anandamide (˜50,000 cpm). Enzyme (CHO cell lysate, brain or liver homogenate) is incubated in assay buffer (50 mM Phosphate, pH 8.0, 1 mM EDTA, 200 mM KCl, 0.2% glycerol, 0.1% fatty acid free BSA) with inhibitor at 25° C. for 30 minutes. The reaction was terminated by addition of 2 volumes of chloroform:methanol (1:1) and mixed by vortexing. Following a centrifugation step, 2000 rpm for 10 mM. at room temperature, the aqueous phase containing the released ³H-ethanolamide was recovered and quantitated by liquid scintillation as a reflection of FAAH enzyme activity.
Ramarao M. K., et al. A fluorescence-based assay for fatty acid amide hydrolase compatible with high-throughput screening. Anal. Biochem. 343:143-51 (2005)
Wilson S. J., et 1. A high-throughput-compatible assay for determining the activity of fatty acid amide hydrolase. Anal Biochem. 318:270-5 (2003).

Each of Examples was tested and found to demonstrate biological activity. Results for specific Examples are provided below. Each of Examples was found to have an 1050 of 10 μM or lower in these assays.

| Example | Human LY (IC50 nm) | Human WC (IC50 nm) |
|---|---|---|
| B4 | 0.5081 | 4.615 |
| A10 | 0.8719 | 7.67 |
| B9 | 1.43 | 3.832 |
| A23 | 1.647 | 16.42 |
| B36 | 1.792 | 9.531 |
| I3 | 2.151 | 10.74 |
| B7 | 2.226 | 18.77 |
| B8 | 2.338 | 10.29 |
| B5 | 2.583 | 13.69 |
| A26 | 2.841 | 18.35 |
| B13 | 2.886 | 12.98 |
| A14 | 2.979 | 35.61 |
| B50 | 3.065 | 8.044 |
| A16 | 3.079 | 24.01 |
| B27 | 3.183 | 29.95 |
| B45 | 3.279 | 15.65 |
| B11 | 3.286 | 10.22 |
| A36 | 3.347 | 28.88 |
| B43 | 3.421 | 18.89 |
| D5 | 3.834 | 9.158 |
| B10 | 3.882 | 20.73 |
| E4 | 4.216 | 12.39 |
| A7 | 4.389 | 21.91 |
| B6 | 4.425 | 11.84 |
| A12 | 5.292 | 26.98 |
| A27 | 5.564 | 123.5 |
| C15 | 5.896 | 9.919 |
| A8 | 5.999 | 21.77 |
| A11 | 6.381 | 84.11 |
| B41 | 6.395 | 16.19 |
| B37 | 6.422 | 21.66 |
| E3 | 6.512 | 16.57 |
| E34 | 7.084 | 9.926 |
| C14 | 7.43 | 28.46 |
| C4 | 7.644 | 47.97 |
| A32 | 7.835 | 15.06 |
| E5 | 8.066 | 53.85 |
| B14 | 9.47 | 47.36 |
| B48 | 9.497 | 29.15 |
| E2 | 9.701 | 23.41 |
| C16 | 9.794 | 18.11 |
| E7 | 9.922 | 18.83 |
| B29 | 10.23 | 41.15 |
| B12 | 11.6 | 27.13 |
| A17 | 11.62 | 153.8 |
| A28 | 11.81 | 31.63 |
| F8 | 12.18 | 69.42 |
| A34 | 12.31 | 17.15 |
| E8 | 12.43 | 72.7 |
| A31 | 13.21 | 42.03 |
| D6 | 13.41 | 32.74 |
| D7 | 14.2 | 22.83 |
| B51 | 14.62 | 73.94 |
| C11 | 15.23 | 107.3 |
| E9 | 15.37 | 82.53 |
| B40 | 15.94 | 68.44 |
| C7 | 16.35 | 127.8 |
| A18 | 16.44 | 28.81 |
| A29 | 16.94 | 132.1 |
| A13 | 17.76 | 73.15 |
| E10 | 18.5 | 70.61 |
| A28 | 20.47 | 60.4 |
| B16 | 21.66 | 119.4 |
| B38 | 22.54 | 351.8 |
| B18 | 23.24 | 123.5 |
| B49 | 23.84 | 66.31 |
| E11 | 26.63 | 71.64 |
| E12 | 28.79 | 76.74 |
| A24 | 30.43 | 128.3 |
| C17 | 30.45 | 100.7 |
| E35 | 31.7 | 115 |
| B17 | 31.86 | 189.9 |
| B35 | 33.52 | 344.1 |
| A25 | 37.55 | 147.5 |
| C8 | 42.87 | 271.7 |
| A15 | 44.11 | 34.39 |
| A35 | 46.25 | 822.4 |
| E13 | 48.88 | 160 |
| F2 | 52.95 | 151.1 |
| A30 | 56.34 | 832.5 |
| J12 | 58.95 | 201.6 |
| B19 | 65.56 | 364.3 |
| J8 | 80.35 | 433.2 |
| B32 | 94.8 | 437.6 |
| E33 | 106 | 185.5 |
| B15 | 107.2 | 1111 |
| A20 | 110.5 | 370.4 |
| B31 | 113.2 | 685.6 |
| B46 | 114.4 | 1408 |
| E14 | 116.1 | 925.4 |
| A22 | 116.7 | 370.4 |
| J6 | 132 | 399.5 |
| A33 | 133.5 | 405.3 |
| E23 | 148.4 | 251.6 |
| E17 | 168.4 | 1298 |
| E18 | 170.9 | 1342 |
| E19 | 181.1 | 1208 |
| A19 | 195.8 | 1743 |
| B30 | 207.8 | 1111 |
| B34 | 212.7 | 1111 |
| B28 | 217.2 | 1524 |
| A21 | 229.9 | 370.4 |
| B20 | 238 | 4165 |
| C9 | 251.2 | 773.4 |
| B47 | 251.8 | 572.7 |
| C13 | 255.6 | 2632 |
| J7 | 260.2 | 1143 |
| E20 | 288.9 | 1040 |
| E21 | 316.8 | 1392 |
| F9 | 339.3 | 1111 |
| B39 | 370.4 | 370.4 |
| B33 | 464.4 | 8543 |
| E22 | 497.1 | 3216 |
| C10 | 660.2 | 1993 |
| J11 | 694.9 | 5892 |
| J10 | 818.1 | 8409 |
| E16 | 2030 | 8488 |
| C12 | 2079 | 2681 |
| J9 | 2595 | 3333 |
| J4 | 5874 | 10000 |
| F7 | | |
| B52 | | 35.63 |
| H7 | | 1200 |
| H8 | | 88.52 |
| F4 | | 266.1 |
| F10 | | 349.9 |
| F6 | | 3470 |
| H6 | | 523.3 |
| B42 | | 5.84 |
| E25 | | 59.48 |
| E26 | | 504.4 |
| G3 | | 672.3 |
| G5 | | 11.07 |
| F3 | | 64.87 |
| H2 | | 152.3 |
| H4 | | 688.8 |
| E27 | | 111.9 |
| C6 | | 1089 |
| B44 | | 24.99 |
| C21 | | 362.6 |
| C20 | | 2107 |
| G7 | | 23.8 |
| H5 | | 31.75 |
| E32 | | 9.347 |
| E29 | | 17.3 |
| E30 | | 87.22 |
| C19 | | 606.6 |
| C18 | | 2975 |
| C22 | | 1168 |
| E31 | | 144.2 |
| E28 | | 24.01 |

### Preparation of the Compounds of the Invention.

The compounds of the present invention can be prepared according to the procedures denoted in the following reaction Schemes and Examples or modifications thereof using readily available starting materials, reagents, and conventional procedures thereof well-known to a practitioner of ordinary skill in the art of synthetic organic chemistry. Specific definitions of variables in the Schemes are given for illustrative purposes only and are not intended to limit the procedures described.

### General Scheme

### Aza-Indole CM

### Experiment Procedures

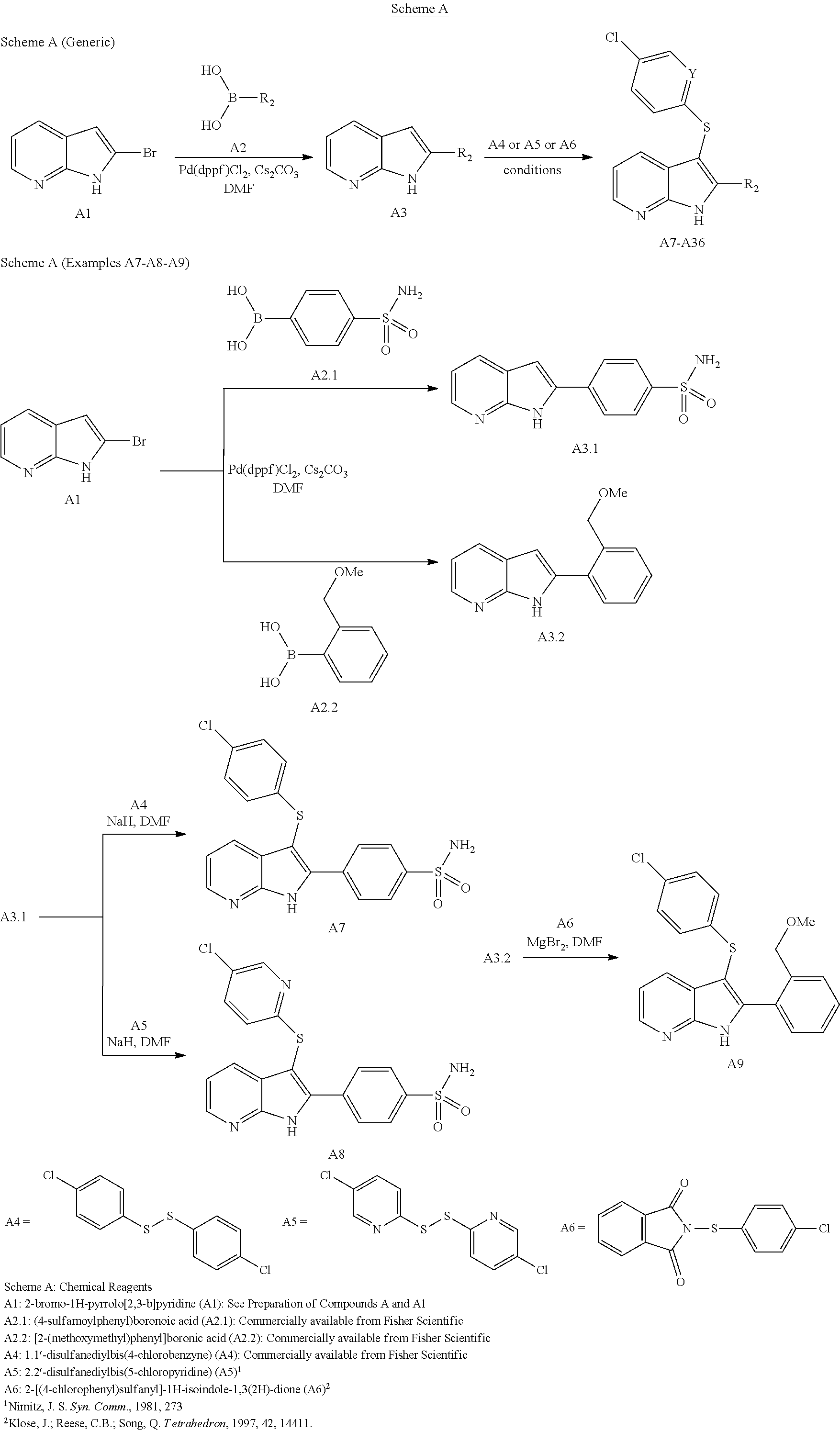

### Preparation of Compounds A and A1

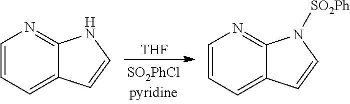

Into a 500 mL, 3-neck, round bottomed flask was placed a solution of 1H-pyrrolo[2,3-b]pyridine (59 g, 500.00 mmol, 1.00 equiv) in THF (500 mL) and pyridine (4 g, 50.63 mmol, 0.10 equiv). To the mixture was added benzenesulfonyl chloride (88 g, 49830 mmol, 1.00 equiv). The resulting solution was allowed to react, with stirring, overnight at room temperature. The reaction mixture was then quenched by adding 500 mL of H₂O. The resulting mixture was extracted two times with 200 mL of EtOAc. The combined organic layers was dried over Na₂SO₄ and concentrated under vacuum using a rotary evaporator. The residue was purified by eluting through a column with a 1:5 EtOAc/PE solvent system. This resulted in 43 g (37%) of 1-(phenylsulfonyl)-1H-pyrrolo[2,3-b]pyridine as a yellow solid.

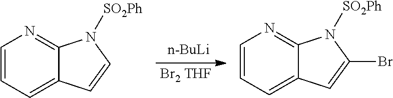

Into a 5000 mL, 4-neck, round bottomed flask, purged and maintained with an inert atmosphere of nitrogen, was placed a solution of 1-(phenylsulfonyl)-1H-pyrrolo[2,3-b]pyridine (85.4 g, 331.01 mmol, 1.00 equiv) in THF (3000 mL). To the above was added n-BuLi (172 mL, 1.10 equiv, 2.5M) drop wise with stirring, while cooling to a temperature of −78° C. The reaction mixture was stirred for 2 hours at −40° C. To the above was added n-BuLi (13.2 mL, 0.10 equiv, 2.5M) drop wise with stirring at −40° C. The reaction was stirred for 1 hour. To the above was added n-BuLi (13.2 mL, 0.10 equiv, 2.5M) drop wise with stirring at −40° C. After stirring for 1 hour, a solution of Br₂ (61 g, 381.25 mmol, 1.45 equiv) in hexane (250 mL) was added drop wise with stirring, while cooling to a temperature of −78° C. The resulting solution was allowed to react, with stirring, for 1 hour at −78° C. The reaction mixture was then quenched by adding 500 mL of H₂O. The resulting solution was extracted with 1000 mL of EtOAc. The EtOAc solution was dried over Na₂SO₄ and concentrated under vacuum using a rotary evaporator. This resulted in 66 g (63%) of 2-bromo-1-(phenylsulfonyl)-1H-pyrrolo[2,3-b]pyridine as a yellow solid.

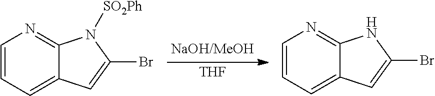

Into a 2000 mL, 4-neck, round bottomed flask was placed a solution of 2-bromo-1-(phenylsulfonyl)-1H-pyrrolo[2,3-b]pyridine (38.7 g, 91.87 mmol, 1.00 equiv, 80%) in THF (950 mL). To this was added NaOH/MeOH (73 mL, 5M). The resulting solution was allowed to react, with stirring, for 30 minutes at room temperature. The reaction mixture was then quenched by adding 2000 mL of H₂O. The resulting solution was diluted with 600 mL of NH₄Cl solution. A filtration was performed. The filter cake was washed 1 time with 200 mL of H₂O, 1 time with 500 mL of hexane, then dried under vacuum. This resulted in 22 g (81%) of 2-bromo-1H-pyrrolo[2,3-b]pyridine as a yellow solid.

LC-MS (ES, m/z): 197 [M+H]⁺, 238 [M+MeCN+H]⁺.

H-NMR (400 MHz, CDCl₃, ppm): 6.55 (1H, s), 7.14-7.27 (1H, m), 7.91-7.93 (1H, d), 8.36 (1H, s).

### Scheme for Compound A

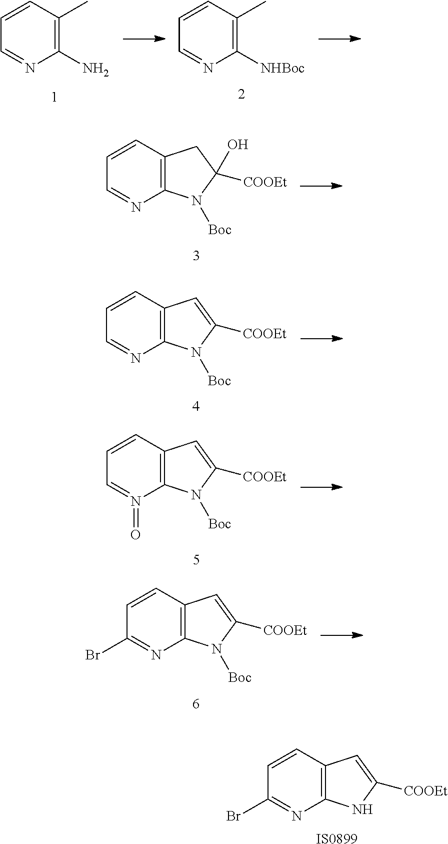

### Synthesis of 2:

A solution of 1 (108 g, 1.0 mol) and (Boc)₂O (239.8 g, 1.11 mol) in THF (650 mL) was heated under reflux with stirring overnight. After cooling, the white solid was filtered and re-crystallized with EA/PE (1:4) to afford 2 (179 g, 86%) as white solid.

### Synthesis of 3:

To a stirred solution of 2 (122 g, 0.59 mol) in THF (0.8 L) at −10° C. under N₂ atmosphere was slowly added a solution of n-BuLi (496 mL of 2.54M in hexane, 1.24 mol) dropwise. The mixture was stirred for 1 h then added a solution of (COOEt)₂ (258 g, 1.77 mol) in 400 mL THF at 0° C. under N₂ atmosphere. The mixture was stirred for 1.5 h and partitioned between water and EA. The aqueous layer was extracted with EA. The combined organic layer were washed with brine, dried with MgSO₄, concentrated in vacuo and purified by column chromatography [EA/PE (v:v)=1:4] to afford 3 (55 g, 30% yield) as yellow solid.

### Synthesis of 4:

A solution of 3 (51 g, 0.165 mol) in DME (500 mL) at 0° C. was stirred and added solution of TFAA (138.6, 0.66 mol) and pyridine (111.4 g, 1.41 mol) in DME (360 mL) at 0° C. The reaction mixture was allowed to warm to room temperature. After the reaction was completed, the reaction mixture was concentrated in vacuo. The residue was suspended in CH₂Cl₂, and extracted with water. The organic phase was dried with MgSO₄, concentrated in vacuo and purified by column chromatography [EA/PE (v:v)=1:8] to afford 4 (41.0 g, 85% yield) as yellow oil.

### Synthesis of 5:

To a solution of 4 (50.0, 0.17 mol) in CH₂Cl₂ (1.3 L) at room temperature was stirred and added m-CPBA (73.0 g, 0.43 mol). The reaction mixture was stirred overnight. Then another m-CPBA (73.0 g, 0.43 mol) batch was added. The mixture was refluxed until a full conversion, then poured into K₂CO₃ solution. The organic layer was washed with Na₂SO₃ solution and brine. The combined organic phase was dried with MgSO₄ and concentrated in vacuo. The crude product was purified by re-crystallization with tert-butyl methyl ether and dried under high vacuum to give the product 5 (24.3 g, 46% yield) as white solid.

### Synthesis of 6:

To a stirred suspension of 5 (61.2 g, 0.2 mol) in toluene (1.8 L) was added simultaneously a solutions of HMDS (32.2 g, 0.2 mol) in toluene (0.5 L) and PhCOBr (90.6 g, 0.49 mol) in toluene (0.5 L) dropwise. After two additional hours, the reaction mixture was poured into Na₂CO₃ solution. The water layer was extracted with EA and the combined organic layer was dried with MgSO₄, concentrated in vacuo and purified by column chromatography [EA/PE (v:v)=1:20] to afford 6 (41.2 g, 55% yield) as white solid.

### Synthesis of Compound A:

A stirred solution of 6 (41.2, 0.11 mol) in DCM (500 mL) was cooled to 0° C. and added TFA (126.9, 1.10 mol) over 10 min. After the reaction was completed detected by TLC, the reaction mixture was poured into Na₂CO₃ solution. The mixture was extracted with DCM. The combined organic phase was washed with brine, dried over MgSO₄ and evaporated to give the product “A” (27.8, 92% yield) as white solid. ¹H NMR (CDCl₃, 300 MHz) δ: 923 (b, 1H), 7.86 (d, J=8.4 Hz, 1H), 7.30 (d, J=8.4 Hz, 1H), 7.15 (d, J=2.4 Hz, 1H), 4.42 (q, J=7.2 Hz, 2H), 1.41 (t, J=7.2 Hz, 3H). LC-MS: 268.9 (M+1)⁺.

### Scheme A: Synthetic Procedures

### Step A3-1: 4-(1H-pyrrolo[2,3-b]pyridin-2-yl)benzenesulfonamide (A3-1)

2-bromo-1H-pyrrolo[2,3-b]pyridine (250 mg, 1.269 mmol), (4-sulfamolyphenyl)boronic acid (503 mg, 1.776 mmol), and cesium carbonate (2538 μL, 2.54 mmol, 1M aqueous solution) were dissolved in DMF (6.4 mL) and the resulting mixture was degassed with nitrogen for 10 minutes. 1,1′-bis(diphenylphosphino)ferrocene-palladium(II)dichloride dichloromethane complex (104 mg, 0.127 mmol) was added and the resulting mixture was heated to 100° C. in a sealed tube for 19 hours. The metal catalyst was scavenged by stirring with QuadraPore for 24 hours and the crude reaction mixture was purified using reverse phase chromatography. The appropriate fractions were lyophilized to afford 73 mg of an off-white solid. ¹H NMR (CDCl₃): δ 7.95 (m, 4H), 7.54 (m, 4H). LCMS (M+1)=274.3.

### Step A3-2: 2-[2-(methoxymethyl)phenyl]-1H-pyrrolo[2,3-b]pyridine

(A3-2): 2-bromo-1H-pyrrolo[2,3-b]pyridine (200 mg, 1.015 mmol), (4-sulfamolyphenyl)boronic acid (236 mg, 1.421 mmol), and cesium carbonate (2030 μL, 2.030 mol, 1M aqueous solution) were dissolved in DMF (2.03 mL) and the resulting mixture was degassed with nitrogen for 10 minutes. 1,1′-bis(diphenylphosphino)ferrocene-palladium(II)dichloride dichloromethane complex (104 mg, 0.127 mmol) was added and the resulting mixture was heated to 100° C. in a sealed tube for 19 hours. The metal catalyst was scavenged by stirring with QuadraPore for 30 hours and the crude reaction mixture was purified using reverse phase chromatography. The appropriate fractions were lyophilized to afford 75 mg of an off-white solid. LCMS (M+1)=239.3.

### Step A7-1: 4-{3-[(4-chlorophenyl)sulfanyl]-1H-pyrrolo[2,3-b]pyridin-2-yl}benzenesulfonamide

(A7): 4-(1H-pyrrolo[2,3-b]pyridin-2-yl)benzenesulfonamide (35 mg, 0.064 mmol) and NaH (95% wt, 3.23 mg, 0.128 mmol) were dissolved in anahydrous DMF (320 μL) at 0° C. and stirred for 5 minutes before the addition of 1,1′-disulfanediylbis(4-chlorobenzene) (46.0 mg, 0.160 mmol). The reaction mixture was allowed to warm to room temperature over 1.5 hours and was quenched with the dropwise addition of 2 mL of water. The crude reaction mixture was syringe filtered and purified by reverse phase chromatography. The appropriate fractions were lyophilized to afford 3.4 mg of a white solid. ¹H NMR (CDCl₃): δ 8.32 (d d, J=3.39 Hz, J=1.46 Hz, 1H), 8.00 (m, 5H), 7.19 (m, 1H), 7.15 (d, J=8.7 Hz, 2H), 6.98 (d, J=8.7 Hz, 2H). LCMS (M+1)=416.3, HRMS Calculated=416.0289, Measured=416.0299.

### Step A8-1: 4-{3-[(5-chloropyridin-2-yl)sulfanyl]-1H-pyrrolo[2,3-b]pyridin-2-yl}benzenesulfonamide (A8)

4-(1H-pyrrolo[2,3-b]pyridin-2-yl)benzenesulfonamide (7 mg, 0.026 mmol) and NaH (95% wt, 1.3 mg, 0.051 mmol) were dissolved in anahydrous DMF (128 μL) at 0° C. and stirred for 5 minutes before the addition of 2,2′-disulfanediylbis(5-chloropyridine) (18.52 mg, 0.064 mmol). The reaction mixture was allowed to warm to room temperature over 7 hours and was quenched with the drop-wise addition of 0.5 mL of water. The crude reaction mixture was syringe filtered and purified by reverse phase chromatography. The appropriate fractions were lyophilized to afford 0.2 mg of a white solid. ¹H NMR (d-DMSO): δ 12.94 (s, 1H), 8.43 (d, J=2.47 Hz, 1H), 8.40 (dd, J=3.20 Hz, J=1.47 Hz, 1H), 8.00 (d, J=8.51 Hz, 2H), 7.91 (d, J=8.51 Hz, 2H), 7.85 (d, J=6.86 Hz, 1H), 7.66 (dd, J=6.13 Hz, J=2.57 Hz, 1H), 7.21 (m, 1H), 6.82 (d, J=8.61 Hz, 1H). LCMS (M+1)=417.3, HRMS Calculated=417.0241 Measured=417.0244.

### Step A9-1: 3-[(4-chlorophenyl)sulfanyl]-2-[2-(methoxymethyl)phenyl]-1H-pyrrolo[2,3-b]pyridine (A9)

2-[2-(methoxymethyl)phenyl]-1H-pyrrolo[2,3-b]pyridine (50 mg, 0.210=101), 2-[(4-chlorophenyl)sulfanyl]-1H-isoindole-1,3(2H)-dione (66.9 mg, 0.231 mmol), and magnesium bromide (19.32 mg, 0.105 mmol) were combined in DMF (1049 μL) and the reaction mixture was heated to 100° C. in a sealed tube for 18 hours. The crude reaction mixture was syringe filtered and purified by reverse phase chromatography. The appropriate fractions were lyophilized to afford 18 mg of a white solid. ¹H NMR (d-DMSO): δ 12.47 (s, 1H), 8.32 (d, J=4.58 Hz, 1H), 7.74 (d, J=7.87 Hz, 1H), 7.50 (d, J=7.69 Hz, 1H), 7.45 (t, J=7.69 Hz, 1H), 7.34 (m, 2H), 7.21 (d, J=8.43 Hz, 2H), 7.15 (d of d, J=7.69 Hz, J=7.76 Hz, 1H), 6.93 (d, J=8.42 Hz, 2H), 4.36 (s, 2H), 3.07 (s, 3H). LCMS (M+1)=381.4, HRMS Calculated=381.0823 Measured=381.0823.

TABLE 1
(Scheme A)

| Compound | Name | |
|---|---|---|
| No. | Structure | HRMS |
| A10 | 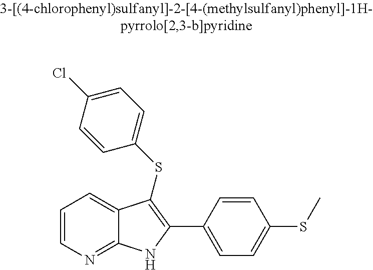 [SMILES: CSc1ccc(-c2[nH]c3ncccc3c2Sc2ccc(Cl)cc2)cc1] | 383.0438 |
| A11 | 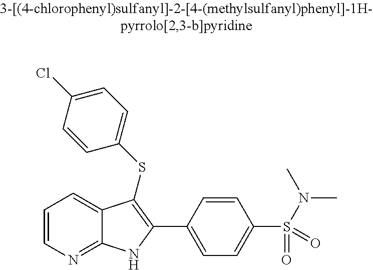 [SMILES: CN(C)S(=O)(=O)c1ccc(-c2[nH]c3ncccc3c2Sc2ccc(Cl)cc2)cc1] | 444.0595 |
| A12 | 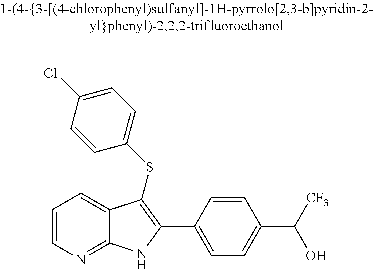 [SMILES: OC(c1ccc(-c2[nH]c3ncccc3c2Sc2ccc(Cl)cc2)cc1)C(F)(F)F] | 435.0541 |
| A13 | 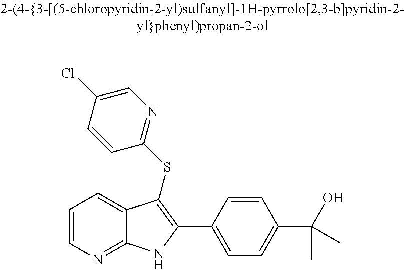 [SMILES: CC(C)(O)c1ccc(-c2[nH]c3ncccc3c2Sc2ccc(Cl)cn2)cc1] | 396.0931 |
| A14 | 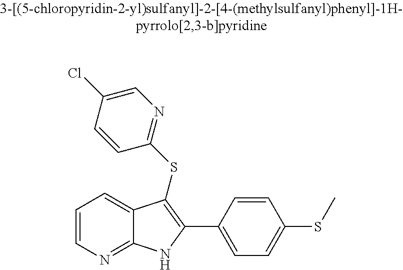 [SMILES: CSc1ccc(-c2[nH]c3ncccc3c2Sc2ccc(Cl)cn2)cc1] | 384.0387 |
| A15 | 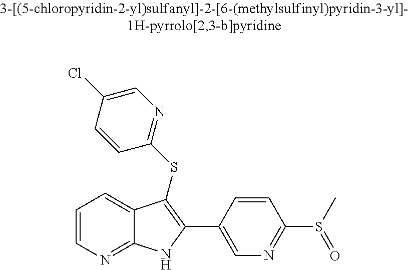 [SMILES: CS(=O)c1ccc(-c2[nH]c3ncccc3c2Sc2ccc(Cl)cn2)cn1] | 401.0292 |
| A16 | 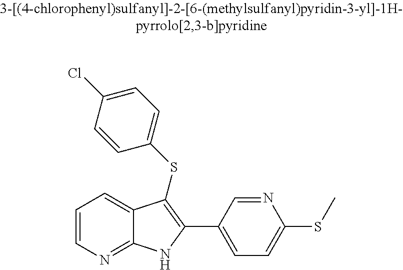 [SMILES: CSc1ccc(-c2[nH]c3ncccc3c2Sc2ccc(Cl)cc2)cn1] | 384.0389 |
| A17 | 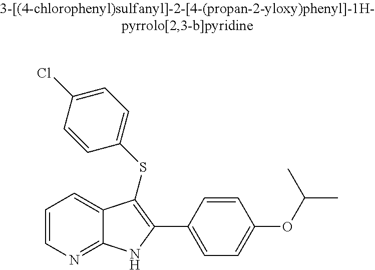 [SMILES: CC(C)Oc1ccc(-c2[nH]c3ncccc3c2Sc2ccc(Cl)cc2)cc1] | 395.0984 |
| A18 | 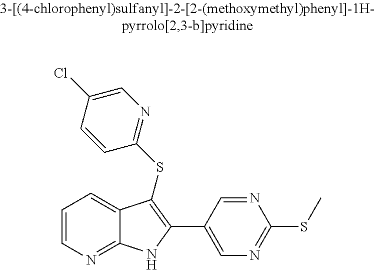 [SMILES: CSc1ncc(-c2[nH]c3ncccc3c2Sc2ccc(Cl)cn2)cn1] | 402.0250 |
| A19 | 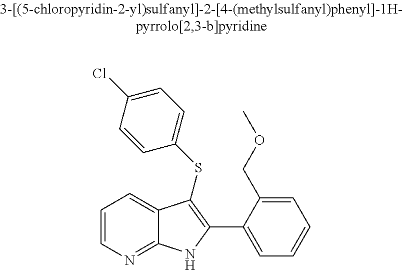 [SMILES: COCc1ccccc1-c1[nH]c2ncccc2c1Sc1ccc(Cl)cc1] | 381.0824 |
| A20 | 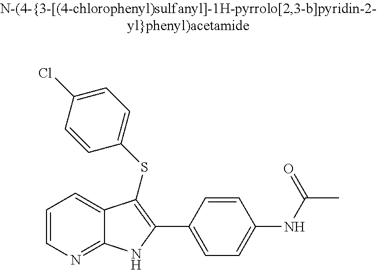 [SMILES: CC(=O)Nc1ccc(-c2[nH]c3ncccc3c2Sc2ccc(Cl)cc2)cc1] | 394.0775 |
| A21 | 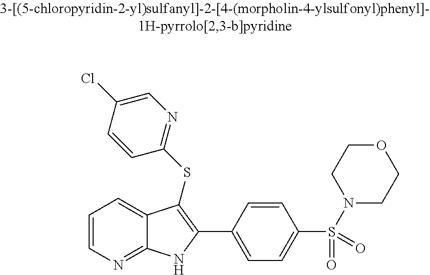 [SMILES: O=S(=O)(c1ccc(-c2[nH]c3ncccc3c2Sc2ccc(Cl)cn2)cc1)N1CCOCC1] | 487.0655 |
| A22 | 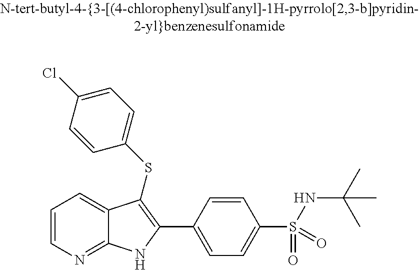 [SMILES: CC(C)(C)NS(=O)(=O)c1ccc(-c2[nH]c3ncccc3c2Sc2ccc(Cl)cc2)cc1] | 472.0919 |
| A23 | 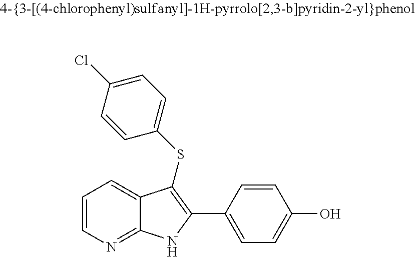 [SMILES: Oc1ccc(-c2[nH]c3ncccc3c2Sc2ccc(Cl)cc2)cc1] | 353.0511 |
| A24 | 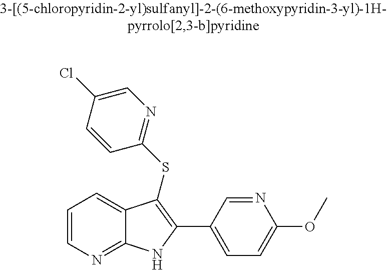 [SMILES: COc1ccc(-c2[nH]c3ncccc3c2Sc2ccc(Cl)cn2)cn1] | 369.0574 |
| A25 | 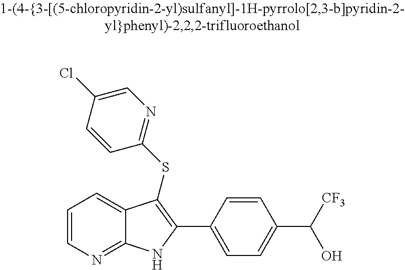 [SMILES: OC(c1ccc(-c2[nH]c3ncccc3c2Sc2ccc(Cl)cn2)cc1)C(F)(F)F] | 436.0495 |
| A26 | 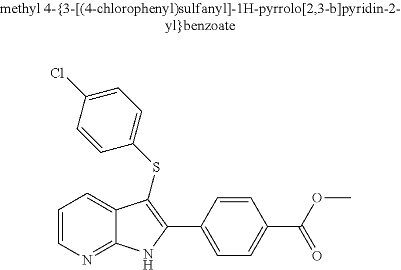 [SMILES: COC(=O)c1ccc(-c2[nH]c3ncccc3c2Sc2ccc(Cl)cc2)cc1] | 395.0617 |
| A27 | 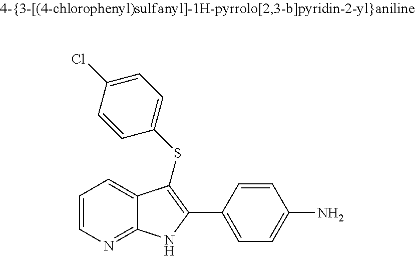 [SMILES: Nc1ccc(-c2[nH]c3ncccc3c2Sc2ccc(Cl)cc2)cc1] | 352.0670 |
| A28 | 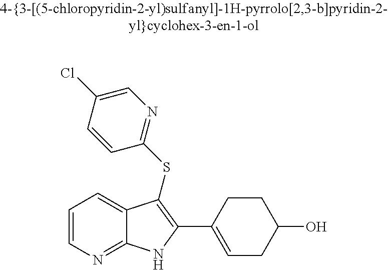 [SMILES: OC1CC=C(c2[nH]c3ncccc3c2Sc2ccc(Cl)cn2)CC1] | 358.0776 |
| A29 | 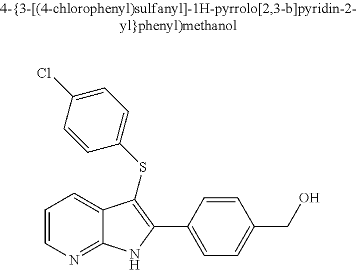 [SMILES: OCc1ccc(-c2[nH]c3ncccc3c2Sc2ccc(Cl)cc2)cc1] | 367.0663 |
| A30 | 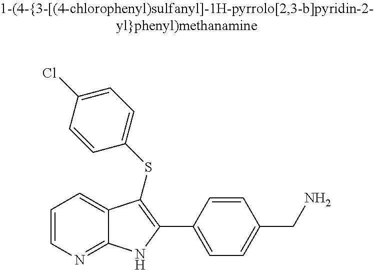 [SMILES: NCc1ccc(-c2[nH]c3ncccc3c2Sc2ccc(Cl)cc2)cc1] | 366.0830 |
| A31 | 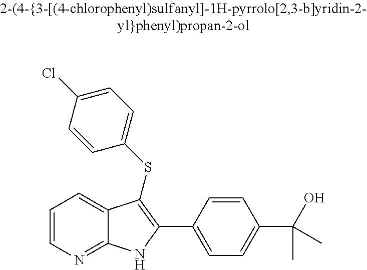 [SMILES: CC(C)(O)c1ccc(-c2[nH]c3ncccc3c2Sc2ccc(Cl)cc2)cc1] | 395.3³ |
| A32 | 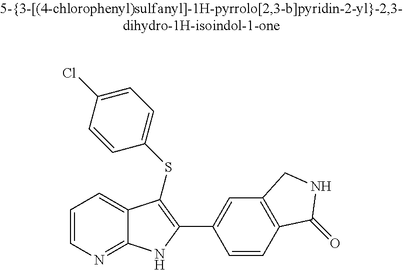 [SMILES: O=C1NCc2cc(-c3[nH]c4ncccc4c3Sc3ccc(Cl)cc3)ccc21] | 392.0629 |
| A33 | 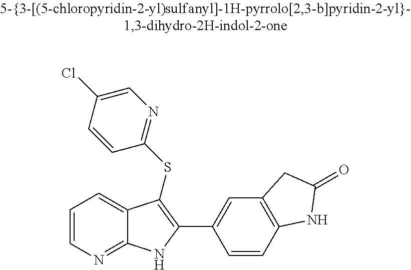 [SMILES: O=C1Cc2cc(-c3[nH]c4ncccc4c3Sc3ccc(Cl)cn3)ccc2N1] | 393.0573 |
| A34 | 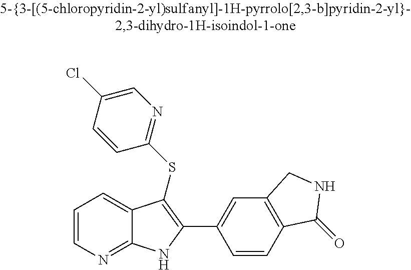 [SMILES: O=C1NCc2cc(-c3[nH]c4ncccc4c3Sc3ccc(Cl)cn3)ccc21] | 393.0579 |
| A35 | 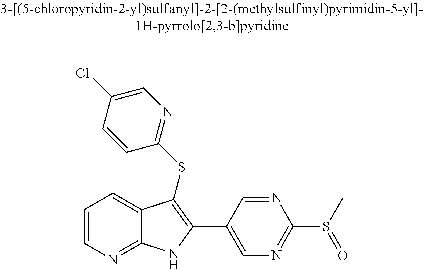 [SMILES: CS(=O)c1ncc(-c2[nH]c3ncccc3c2Sc2ccc(Cl)cn2)cn1] | 386.0300 |
| A36 | 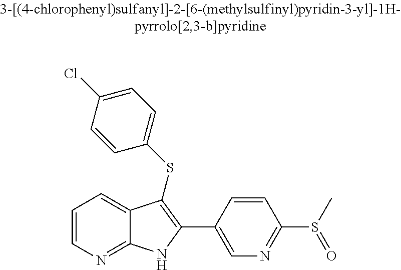 [SMILES: CS(=O)c1ccc(-c2[nH]c3ncccc3c2Sc2ccc(Cl)cc2)cn1] | 400.0344 |

³LCMS data

Compounds A15 & A35 & A36 require an additional oxidation step: Synthetic procedure is as follows for A36 (3-[(4-chlorophenyl)sulfanyl]-2-[6-(methylsulfinyl)pyridin-3-yl]-1H-pyrrolo[2,3-b]pyridine): To a stirring slurry of 3-[(5-chloropyridin-2-yl)sulfanyl]-2-[4-(methylsulfinyl)phenyl]-1H-pyrrolo[2,3-b]pyridine (A16) (1078 mg, 3.00 mmol) in DCM (14 mL) under nitrogen atmosphere, mCPBA (738 mg, 3.00 mmol, 25 mg/mL DCM) was added drop-wise. After 40 minutes, the solution became homogeneous and the crude reaction mixture was concentrated. The crude mixture was purified by reverse phase chromatography and the appropriate fractions were collected and lyophilized to afford 574 mg of a white solid. ¹H NMR (d-DMSO): δ 13.04 (s, 1H), 9.08 (d, J=1.55 Hz, 1H), 8.53 (dd, J=5.95 Hz, J=2.20 Hz, 1H), 8.41 (dd, J=3.11 Hz, J=1.56 Hz, 1H), 8.05 (d, J=7.98 Hz, 1H), 7.87 (d of d, J=6.59 Hz, J=1.37 Hz, 1H), 7.29 (d, J=8.70 Hz, 2H), 7.23 (m, 1H), 7.05 (d, J=8.61 Hz, 2H), 2.85 (s, 3H). LCMS (M−1-1)=400.3. HRMS (M+1)=400.0343. Chiral separation using OD-H, 3 cm×25 cm, with 35% methanol in carbon dioxide. Peak 1 retention time 7.166 min. HRMS Calculated=400.0340 Measured=400.0343. Peak 2 retention time 8.374 min. HRMS Calculated=400.0340 Measured=400.0344.

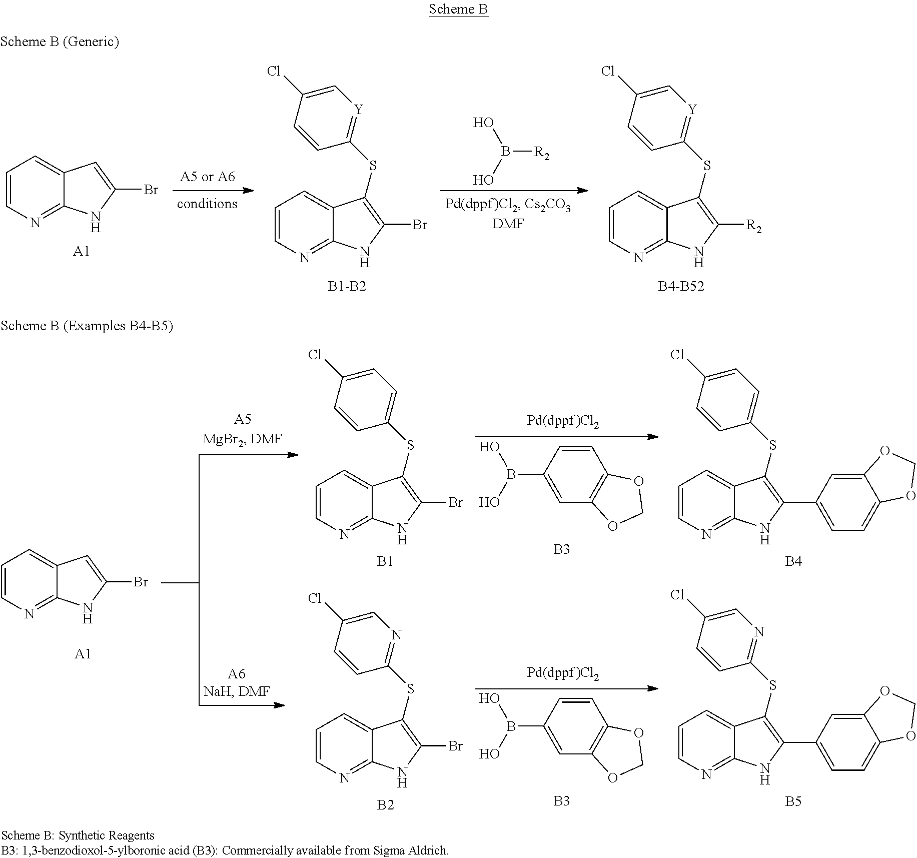

### Scheme B: Synthetic Procedures

### Step B1-1: 2-bromo-3-[(4-chlorophenyl)sulfanyl]-1H-pyrrolo[2,3-b]pyridine (B1)

2-bromo-7-azaindole (1.026 g; 5.2 mmol), A5 (1.66 g; 5.75 mmol) and Magnesium bromide (40 mg; 0.2171=01) was dissolved in DMAc (10 mL) and heated to 60-70° C. for 3 hours under a nitrogen atmosphere. The reaction mixture was then cooled back to ambient temperature. Aqueous Sodium hydroxide (1.0N; 10 mL) was added slowly via addition funnel during which time the product precipitated out as a white solid. The resulting slurry was cooled to ˜10 C and aged for 30 min prior to filtration. The slurry was then filtered at 10 C, washed with water (2×20 mL) and subsequently dried on the filter funnel under a stream of nitrogen to afford 1.7 g of white solid. ¹H NMR (CDCl₃): δ 8.40 (d, J=4.8 Hz, 1H), 7.91 (d, J=8.0 Hz, 1H), 7.20 (dd, J=8.0, 4.8 Hz, 1H), 7.16 (d, J=8.4 Hz, 2H), 7.15 (dd, J=8.8 Hz, 2H), LCMS (M+1)=338.5

### Step B2-1: 2-bromo-3-[(5-chloropyridin-2-yl)sulfanyl]-1H-pyrrolo[2,3-b]pyridine (B2)

2-bronco-7-azaindole (0.5 g; 2.54 mmol) and A6 (0.81 g; 2.79 mmol; 1.1 eq) was dissolved in DMF (10 mL). Sodium hydride (0.31 g; 7.61 mmol; 3 eq; 60 wt % in mineral oil) was then added and the resulting solution was heated to 40° C. for 3 hours under a nitrogen atmosphere. The crude reaction mixture was cooled back to ambient temperature and water (20 mL0 was added during which time product precipitated as a white solid. The crude product was filtered, washed with water (2×20 mL) and purified by silica gel chromatography to yield 200 mg of a white solid. ¹H NMR (CDCl₃): δ 8.45 (dd, J=4.8, 3.6 Hz, 1H), 8.38 (d, J=2.4 Hz, 1H), 7.94 (dd, J=7.6, 1.2 Hz, 1H), 7.38 (dd, J=8.4, 2.4 Hz, 1H), 7.22 (dd, J=7.6, 4.8 Hz, 1H), 6.71 (d, J=8.8 Hz, 1H), LCMS (M+1)=339.5

### Step B4-1: 2-(1,3-benzodioxol-5-yl)-3-[(4-chlorophenyl)sulfanyl]-1H-pyrrolo[2,3-b]pyridine (B4)

2-bromo-3-[(4-chlorophenyl)sulfanyl]-1H-pyrrolo[2,3-h]pyridine (B1) (50 mg, 0.15 mmol), cesium carbonate (96 mg, 0.294 mmol), 1,3-benzodioxol-5-ylboronic acid (B3) (48 mg, 0.29 mmol), and PdCl₂(dppf)CH₂Cl₂ (12 mg, 0.015 mmol) were dissolved in a degassed solution of tetrahydrofuran:water (2:1, 1.5 mL) and placed under argon atmosphere. The resulting solution was heated to 100° C. for 0.5 hours using microwave irradiation. The crude reaction mixture was then filtered over a celite pad, diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 34 mg of a white solid. ¹H NMR (CDCl₃): δ 8.31 (dd, J=4.7 Hz, 1.3 Hz, 1H), 7.78 (dd, J=7.8 Hz, 1.3 Hz, 1H), 7.42 (m, 2H), 7.28 (d, J=7.5 Hz, 2H), 7.15 (dd, J=7.8 Hz, 4.7 Hz, 1H), 7.05 (d, J=8.6 Hz, 1H), 7.00 (d, J=7.5 Hz, 2H), 6.05 (s, 2H). LCMS (M+1)=381.3, HRMS Calculated=381.0459, Measured=381.0456

### Step B5-1: 2-(1,3-benzodioxol-5-yl)-3-[(5-chloropyridin-2-yl)sulfanyl]-1H-pyrrolo[2,3-b]pyridine (B5)

2-bromo-3-[(5-chloropyridin-2-yl)sulfanyl]-1H-pyrrolo[2,3-b]pyridine (32) (50 mg, 0.14 mmol), cesium carbonate (96 mg, 0.294 mmol), 1,3-benzodioxol-5-ylboronic acid (133) (48 mg, 0.29 mmol), and PdCl₂(dppf)CH₂Cl₂ (12 mg, 0.015 mmol) were dissolved in a degassed solution of tetrahydrofuran:water (2:1, 1.5 mL) and placed under argon atmosphere. The resulting solution was heated to 100° C. for 0.5 hours using microwave irradiation. The crude reaction mixture was then filtered over a celite pad, diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 34 mg of a white solid. ¹H NMR (CDCl₃): δ 8.44 (d, J=2.5 Hz, 1H), 8.33 (d, J=4.8 Hz, 1H), 7.81 (d, J=7.8 Hz, 1H), 7.66 (dd, J=8.6 Hz, 2.5 Hz, 1H), 7.37 (m, 2H), 7.18 (dd, J=7.8 Hz, 4.8 Hz, 1H), 7.06 (d, J=8.6 Hz, 1H), 6.78 (d, J=8.6 Hz, 1H), 6.09 (s, 2H). LCMS (M+1)=382.2, HRMS Calculated=382.0412, Measured=−382.0410

TABLE 2
(Scheme B)

| Compound | Name | |
|---|---|---|
| No. | Structure | HRMS |
| B6 | 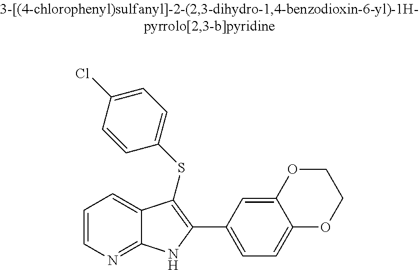 [SMILES: Clc1ccc(Sc2c(-c3ccc4c(c3)OCCO4)[nH]c3ncccc23)cc1] | 395.0620 |
| B7 | 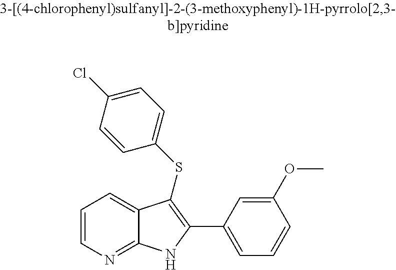 [SMILES: COc1cccc(-c2[nH]c3ncccc3c2Sc2ccc(Cl)cc2)c1] | 367.0662 |
| B8 | 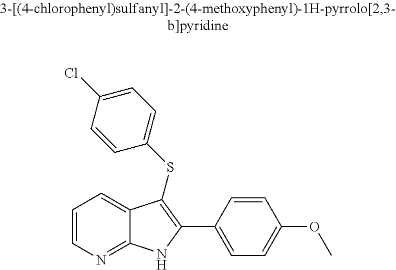 [SMILES: COc1ccc(-c2[nH]c3ncccc3c2Sc2ccc(Cl)cc2)cc1] | 367.0660 |
| B9 | 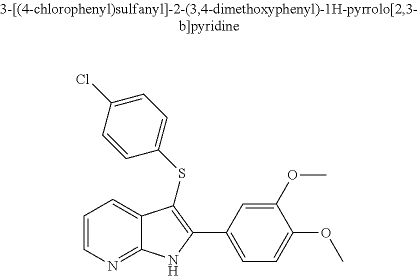 [SMILES: COc1ccc(-c2[nH]c3ncccc3c2Sc2ccc(Cl)cc2)cc1OC] | 397.0768 |
| B10 | 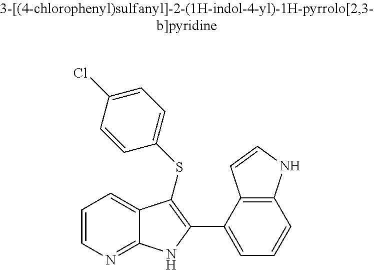 [SMILES: Clc1ccc(Sc2c(-c3cccc4[nH]ccc34)[nH]c3ncccc23)cc1] | 376.0668 |
| B11 | 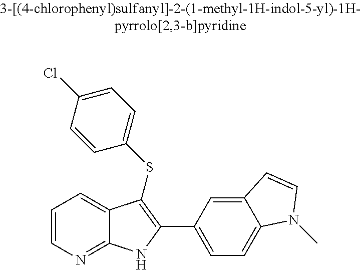 [SMILES: Cn1ccc2cc(-c3[nH]c4ncccc4c3Sc3ccc(Cl)cc3)ccc21] | 390.0826 |
| B12 | 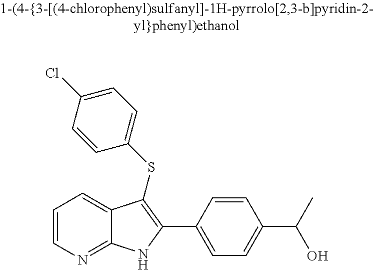 [SMILES: CC(O)c1ccc(-c2[nH]c3ncccc3c2Sc2ccc(Cl)cc2)cc1] | 381.3⁴ |
| B13 | 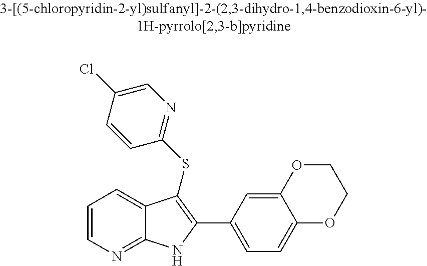 [SMILES: Clc1ccc(Sc2c(-c3ccc4c(c3)OCCO4)[nH]c3ncccc23)nc1] | 396.0570 |
| B14 | 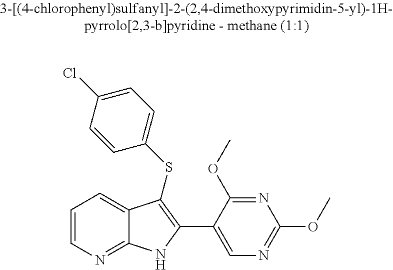 [SMILES: COc1ncc(-c2[nH]c3ncccc3c2Sc2ccc(Cl)cc2)c(OC)n1] | 399.0680 |
| B15 | 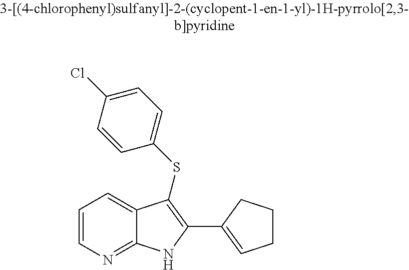 [SMILES: Clc1ccc(Sc2c(C3=CCCC3)[nH]c3ncccc23)cc1] | 327.0722 |
| B16 | 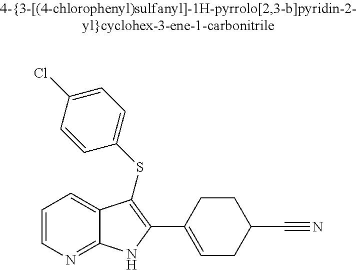 [SMILES: N#CC1CC=C(c2[nH]c3ncccc3c2Sc2ccc(Cl)cc2)CC1] | 366.0828 |
| B17 | 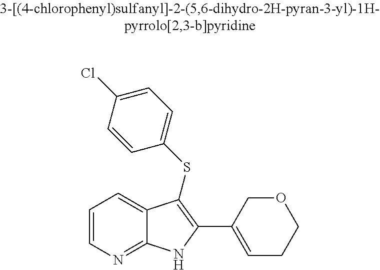 [SMILES: Clc1ccc(Sc2c(C3=CCCOC3)[nH]c3ncccc23)cc1] | 343.0669 |
| B18 | 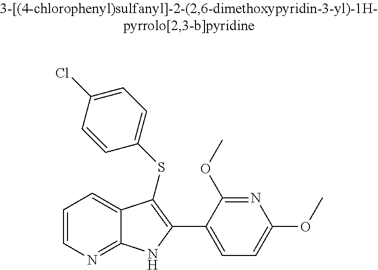 [SMILES: COc1ccc(-c2[nH]c3ncccc3c2Sc2ccc(Cl)cc2)c(OC)n1] | 398.0730 |
| B19 | 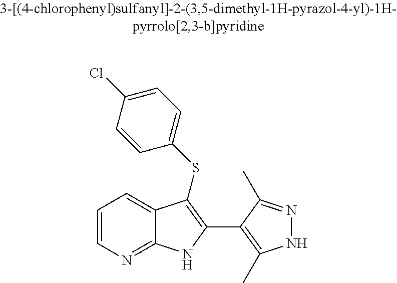 [SMILES: Cc1n[nH]c(C)c1-c1[nH]c2ncccc2c1Sc1ccc(Cl)cc1] | 355.0784 |
| B20 | 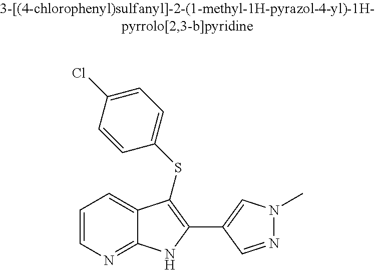 [SMILES: Cn1cc(-c2[nH]c3ncccc3c2Sc2ccc(Cl)cc2)cn1] | 341.0624 |
| B27 | 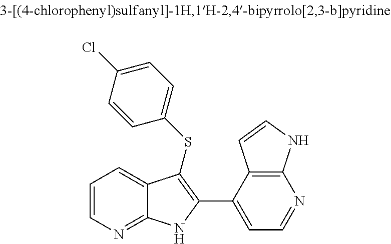 [SMILES: Clc1ccc(Sc2c(-c3ccnc4[nH]ccc34)[nH]c3ncccc23)cc1] | 377.0624 |
| B28 | 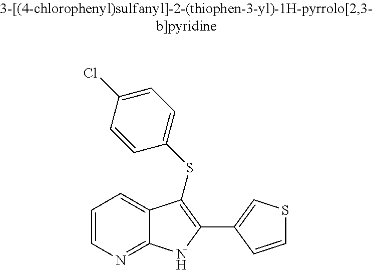 [SMILES: Clc1ccc(Sc2c(-c3ccsc3)[nH]c3ncccc23)cc1] | 343.0124 |
| B29 | 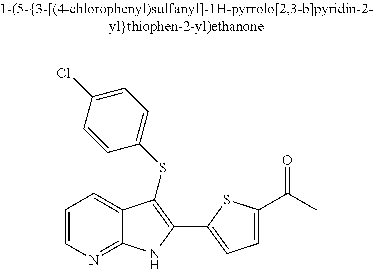 [SMILES: CC(=O)c1ccc(-c2[nH]c3ncccc3c2Sc2ccc(Cl)cc2)s1] | 385.0233 |
| B30 | 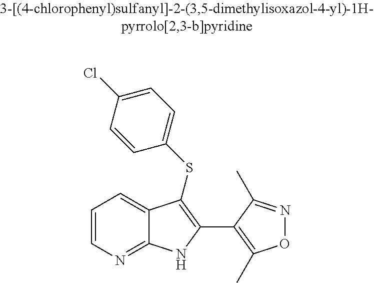 [SMILES: Cc1noc(C)c1-c1[nH]c2ncccc2c1Sc1ccc(Cl)cc1] | 356.0623 |
| B31 | 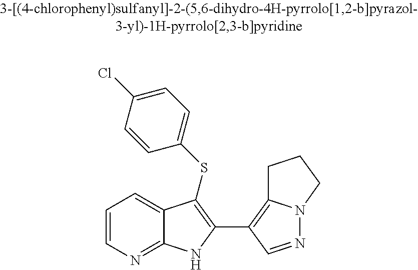 [SMILES: Clc1ccc(Sc2c(-c3cnn4c3CCC4)[nH]c3ncccc23)cc1] | 367.0777 |
| B32 | 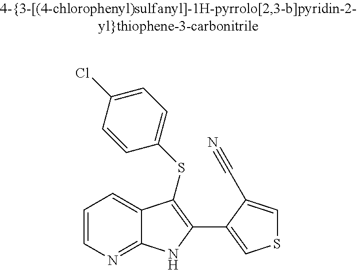 [SMILES: N#Cc1cscc1-c1[nH]c2ncccc2c1Sc1ccc(Cl)cc1] | 368.0081 |
| B33 | 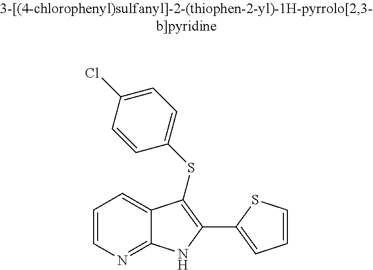 [SMILES: Clc1ccc(Sc2c(-c3cccs3)[nH]c3ncccc23)cc1] | 343.0124 |
| B34 | 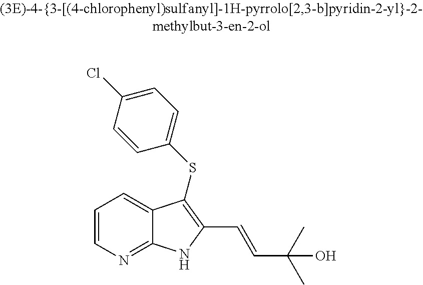 [SMILES: CC(C)(O)/C=C/c1[nH]c2ncccc2c1Sc1ccc(Cl)cc1] | 345.0828 |
| B35 | 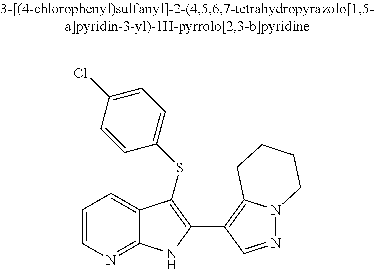 [SMILES: Clc1ccc(Sc2c(-c3cnn4c3CCCC4)[nH]c3ncccc23)cc1] | 381.0939 |
| B36 | 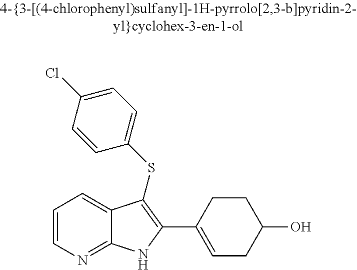 [SMILES: OC1CC=C(c2[nH]c3ncccc3c2Sc2ccc(Cl)cc2)CC1] | 357.0824 |
| B37 | 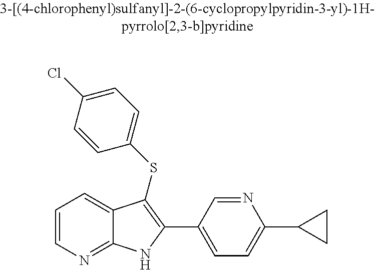 [SMILES: Clc1ccc(Sc2c(-c3ccc(C4CC4)nc3)[nH]c3ncccc23)cc1] | 378.0829 |
| B38 | 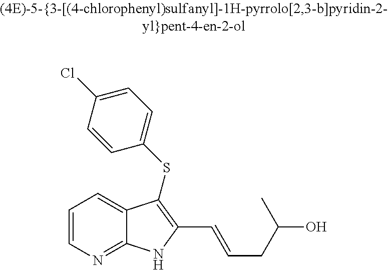 [SMILES: CC(O)C/C=C/c1[nH]c2ncccc2c1Sc1ccc(Cl)cc1] | 345.0827 |
| B39 | 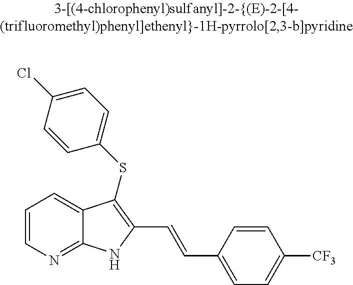 [SMILES: FC(F)(F)c1ccc(/C=C/c2[nH]c3ncccc3c2Sc2ccc(Cl)cc2)cc1] | 431.0595 |
| B40 | 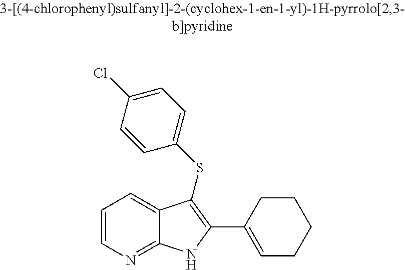 [SMILES: Clc1ccc(Sc2c(C3=CCCCC3)[nH]c3ncccc23)cc1] | 341.0879 |
| B41 | 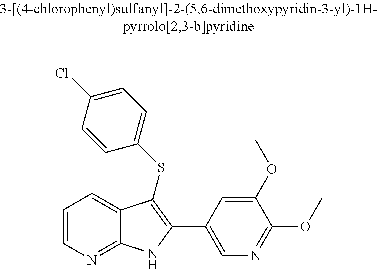 [SMILES: COc1cc(-c2[nH]c3ncccc3c2Sc2ccc(Cl)cc2)cnc1OC] | 398.0735 |
| B42 | 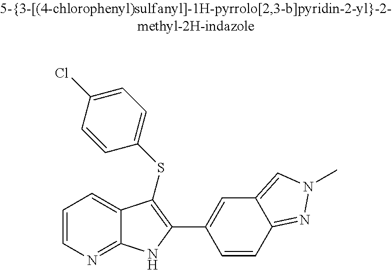 [SMILES: Cn1cc2cc(-c3[nH]c4ncccc4c3Sc3ccc(Cl)cc3)ccc2n1] | 391.0773 |
| B43 | 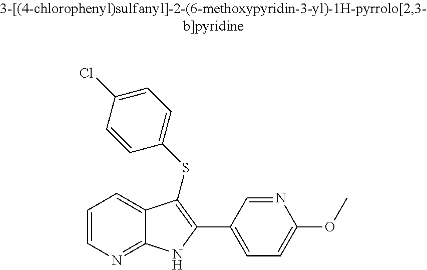 [SMILES: COc1ccc(-c2[nH]c3ncccc3c2Sc2ccc(Cl)cc2)cn1] | 368.0616 |
| B44 | 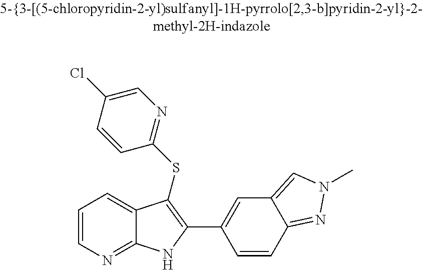 [SMILES: Cn1cc2cc(-c3[nH]c4ncccc4c3Sc3ccc(Cl)cn3)ccc2n1] | 392.0733 |
| B45 | 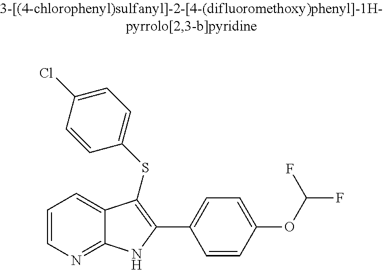 [SMILES: FC(F)Oc1ccc(-c2[nH]c3ncccc3c2Sc2ccc(Cl)cc2)cc1] | 403.0479 |
| B46 | 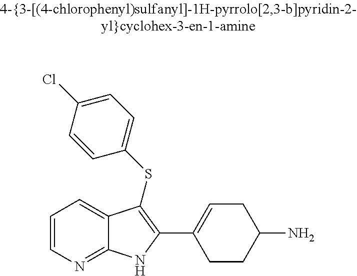 [SMILES: NC1CC=C(c2[nH]c3ncccc3c2Sc2ccc(Cl)cc2)CC1] | 356.0986 |
| B47 | 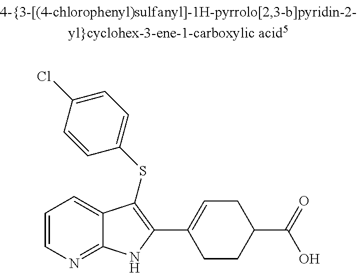 [SMILES: O=C(O)C1CC=C(c2[nH]c3ncccc3c2Sc2ccc(Cl)cc2)CC1] | 385.0767 |
| B48 | 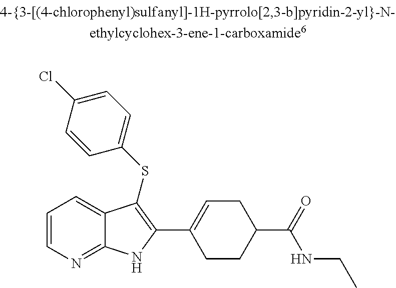 [SMILES: CCNC(=O)C1CC=C(c2[nH]c3ncccc3c2Sc2ccc(Cl)cc2)CC1] | 412.1254 |
| B49 | 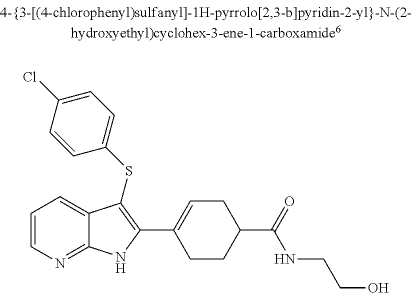 [SMILES: O=C(NCCO)C1CC=C(c2[nH]c3ncccc3c2Sc2ccc(Cl)cc2)CC1] | 428.1195 |
| B50 | 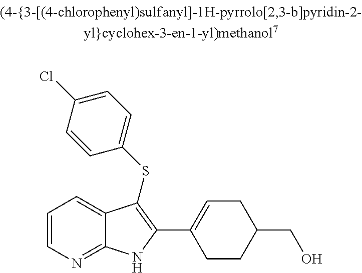 [SMILES: OCC1CC=C(c2[nH]c3ncccc3c2Sc2ccc(Cl)cc2)CC1] | 371.0985 |
| B51 | 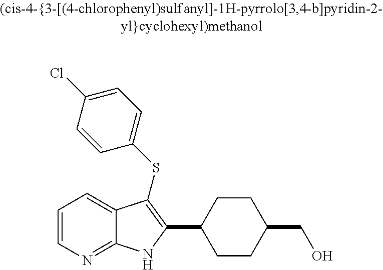 | 373.1137 |
| B52 | 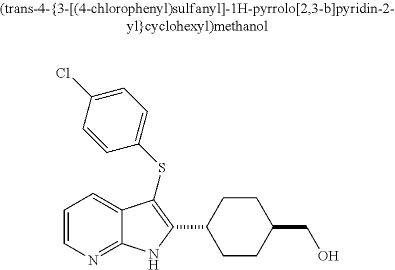 | 373.1137 |

⁴LCMS data
⁵Prepared via hydrolysis of methyl ester. For standard procedure see: Step C2-1
⁶Prepared from B47 via amide coupling reaction. For standard procedure see: Step C6-1

Compounds B51 & B52 require an additional hydrogenation step: Representative synthetic procedure is as follows for (B52): To a solution of (4-{3-[(4-chlorophenyl)sulfanyl]-1H-pyrrolo[2,3-b]pyridin-2-yl}cyclohex-3-en-1-yl)methanol (B50) (50 mg, 0.135 mmol) in ethanol (5 mL) was added PtO₂ (90%) (10 mmol %) and placed on the Parr hydrogenator at 35 psi for 2 days. The crude mixture (1:1 mixture of diastereomers) was purified by reverse phase chromatography and the appropriate fractions were collected and lyophilized to afford 20 mg of a white solid.

(B51) cis: ¹H NMR (CD₃OD) δ 8.25 (dd, J=5.4, 1.5 Hz, 1H), 8.05 (dd, J=8.0, 2.0 Hz, 1H), 7.73 (d, J=7.7 Hz, 1H), 7.25 (dd, J=7.9, 5.3 Hz, 1H), 7.17 (d, J=8.7 Hz, 2H), 6.98 (d, J=8.8 Hz, 1H), 3.71 (d, J=6 Hz, 1H), 3.21 (br. m, 2H), 1.90-1.52 (m, 8H), LCMS (M+1)=373.3, HRMS Calculated=373.1136, Measured=373.1137

(B52) trans: ¹H NMR (CD₃OD) δ 8.23 (dd, J=5.3, 1.4 Hz, 1H), 8.00 (dd, J=7.8, 1.4 Hz, 1H), 7.73 (d, J=7.8 Hz, 1H), 7.25 (dd, J=7.9, 5.3 Hz, 1H), 7.14 (d, J=8.7 Hz, 2H), 6.95 (d, J=8.8 Hz, 2H), 3.38 (d, J=6 Hz 1H), 3.21 (br. m, 2H), 1.90 (m, 6H), 1.60 (m, 1H), 1.10 (m, 1H). LCMS (M+1)=373.3, HRMS Calculated=373.1136, Measured=373.1137

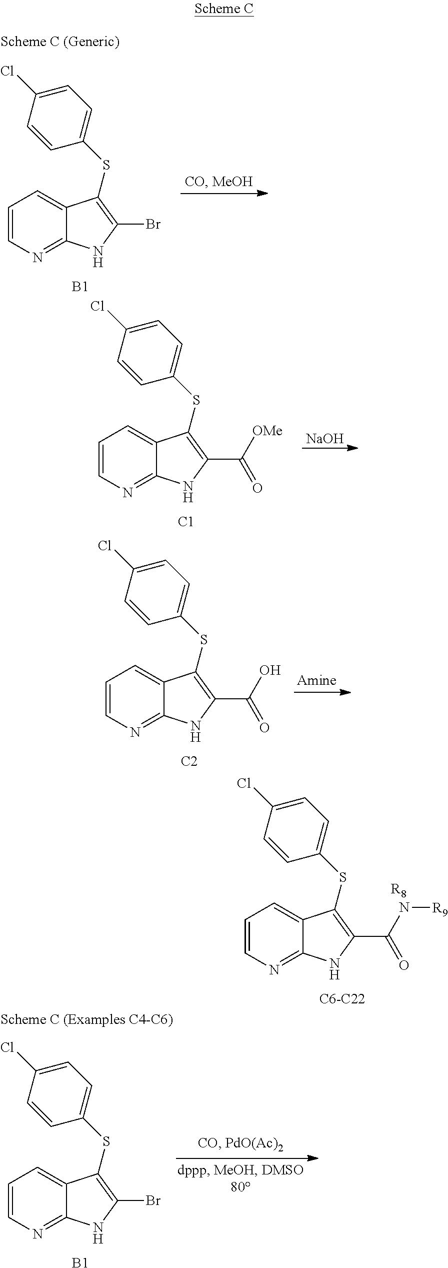

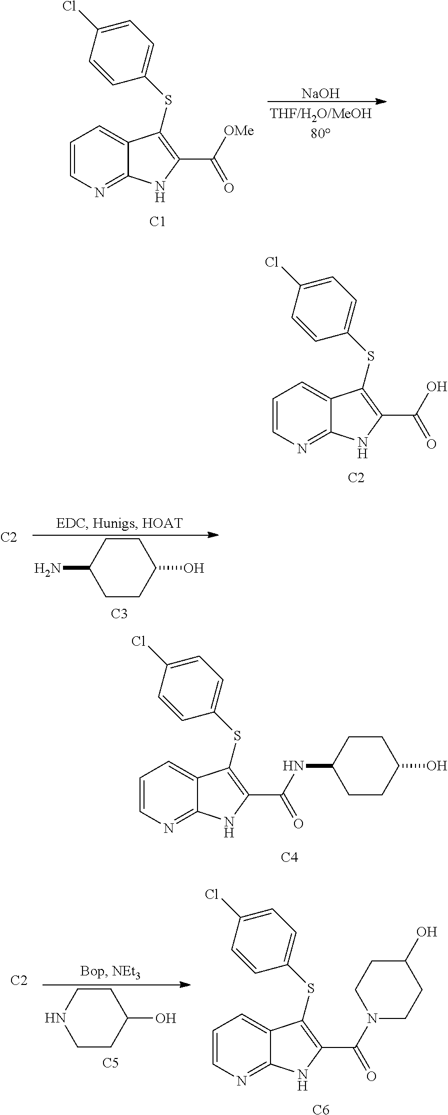

### Scheme C: Synthetic Reagents

C3: trans-4-aminocyclohexanol (C3): Commercially available from Sigma Aldrich.
C5: 4-hydroxy piperidne (C5): Commercially available from Sigma Aldrich.

### Scheme C: Synthetic Procedures

### Step C methyl 3-[(4-chlorophenyl)sulfanyl]-1H-pyrrolo[2,3-b]pyridine-2-carboxylate (C1)

MeOH (58.9 ml) and DMSO (29.4 ml) were degassed with N₂. 2-bromo-3-[(4-chlorophenyl)sulfanyl]-1H-pyrrolo[2,3-b]pyridine (B1) (3.0 g, 8.83 mmol), palladium(II) acetate (0.397 g, 1.767 mmol), triethylamine (4.92 ml, 35.3 mmol), and 1,3-bis(diphenylphosphono)propane (0.729 g, 1.767 mmol) were added to the degassed solvents, and the entire reaction mixture was degassed with N₂. The flask was then fitted with an air condenser and placed under balloon CO atm. The reaction flask was vacuum purged with CO 3×. The reaction was then heated to 80 deg overnight. The reaction mixture was then cooled and diluted with EtOAc and 3M LiCl. The layers were separated and the organic layer was washed with 3M LiCl (2×) and brine. The organic layer was dried over sodium sulfate, filtered and concentrated to yield a brown oil. This brown oil was taken up in dichloromethane and heated. The mixture is allowed to cool and precipitated solid is filtered off to yield 150 mg of pure product. LCMS (M+1)=319.2

### Step C2-1: 3-[(4-chlorophenyl)sulfanyl]-1H-pyrrolo[2,3-b]pyridine-2-carboxylic acid (C2)

Methyl 3-[(4-chlorophenyl)sulfanyl]-1H-pyrrolo[2,3-b]pyridine-2-carboxylate (C1) (500 mg, 1.568 mmol) was dissolved in water (2614 n1), THF (2614 tap and MeOH (2614 n1). NaOH (338 mg, 8.45 mmol) was added and the reaction mixture was heated to 80° for 1 hour. The reaction mixture was cooled and diluted with EtOAc and 1N HCl (8.45 ml) to neutralize to pH=7. The layers were separated and the organic layer was filtered to yield 175 mg of product. The filtrate was then washed with brine, dried with sodium sulfate, filtered and concentrated to yield 300 mg of pure product which was combined with the filtered solid to yield 475 mg of a tan solid. LCMS (M+1)=305.1

### Step C4-1: 3-[(4-chlorophenyl)sulfanyl]-N-(trans-4-hydroxycyclohexyl)-1H-pyrrolo[2,3-b]pyridine-2-carboxamide (C4)

3-[(4-chlorophenyl)sulfanyl]-1H-pyrrolo[2,3-b]pyridine-2-carboxylic acid (C2) (25 mg, 0.082 mmol) and trans 4-aminocyclohexanol (C3) (28.3 mg, 0.246 mmol) were stirred in DMF (820 n1). N,N diisopropylethylamine (43.01, 0.246 mmol), HOAT (11.17 mg, 0.082 mmol), and EDC (18.87 dig, 0.098 mmol) were added and the reaction is stirred for 1 hour. The reaction mixture is filtered through a syringe filter and purified by reverse phase chromatography (5%/95% ACN/H20 to 95%/5% ACN/H₂O over 10 min). Pure fractions were placed on the lyophilizer overnight to yield a white solid. NMR (DMSO) δ 8.40 (d, J=4.39 Hz, 1H), 8.14 (d, J=7.7 Hz, 1H), 7.85 (d, J=7.7 Hz, 1H), 7.29 (d, J=8.6 Hz, 2H), 7.18 (dd, J=7.7 Hz, 3.3 Hz, 1H), 7.06 (d, J=8.6 Hz, 2H), 3.722 (br. m, 1H), 3.39 (br. m, 1H), 1.77-1.84 (m, 4H), 1.225-1.248 (m, 4H). LCMS (M+1)=402.1, HRMS Calculated=402.1038, Measured=402.1054

### Step C6-1 {3-[(4-chlorophenyl)sulfanyl]-1H-pyrrolo[2,3-b]pyridin-2-yl}(4-hydroxypiperidin-1-yl)methanone (C6)

3-[(4-chlorophenyl)sulfanyl]-1H-pyrrolo[2,3-b]pyridine-2-carboxylic acid (C2) (15 mg, 0.049 mmol) and 4-hydroxy piperidine (4.98 mg, 0.049 mmol) appropriate amine were stirred in DMF (492 μl). BOP (32.7 mg, 0.074 mmol) and triethylamine (20.58 μl, 0.148 mmol) were added and the reaction is stirred at RT. After 10 minutes, the reaction mixture was filtered through a syringe filter and purified by reverse phase chromatography. Pure fractions were combined and diluted with EtOAC and saturated sodium bicarbonate. The layers were separated and the organic layer was washed with brine, dried over sodium sulfate, filtered and concentrated to yield a white solid. ¹H NMR (CDCl₃) δ 8.46 (br. s, 1H), 7.84 (d, J=7.8 Hz, 1H); 7.15 (d, J=8.5 Hz, 2H), 4.19 (br. s, 1H), 4.00 (br. s, 1H), 3.39 (m, 2H), 1.95 (br. m, 2H), 1.57 (br.s, 2H). LCMS (M+1)=388.1, HRMS Calculated=388.0881, Measured=388.088

TABLE 3
(Scheme C)⁸

| Com- | | |
|---|---|---|
| pound | Name | |
| No. | Structure | HRMS |
| C7 | 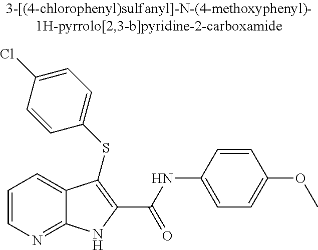 [SMILES: COc1ccc(NC(=O)c2[nH]c3ncccc3c2Sc2ccc(Cl)cc2)cc1] | 426.0496 |
| C8 | 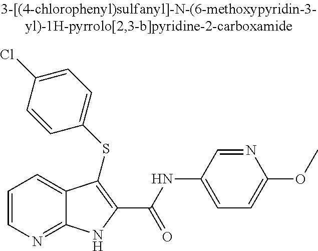 [SMILES: COc1ccc(NC(=O)c2[nH]c3ncccc3c2Sc2ccc(Cl)cc2)cn1] | 411.0678 |
| C9 | 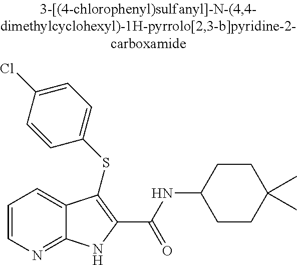 [SMILES: CC1(C)CCC(NC(=O)c2[nH]c3ncccc3c2Sc2ccc(Cl)cc2)CC1] | 414.1404 |
| C10 | 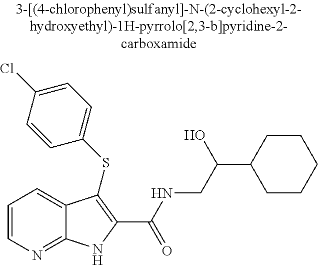 [SMILES: O=C(NCC(O)C1CCCCC1)c1[nH]c2ncccc2c1Sc1ccc(Cl)cc1] | 430.1358 |
| C11 | 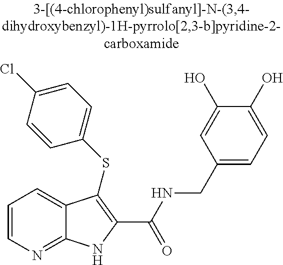 [SMILES: O=C(NCc1ccc(O)c(O)c1)c1[nH]c2ncccc2c1Sc1ccc(Cl)cc1] | 426.0673 |
| C12 | 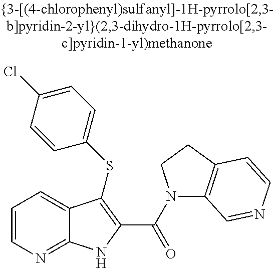 [SMILES: O=C(c1[nH]c2ncccc2c1Sc1ccc(Cl)cc1)N1CCc2ccncc21] | 407.0730 |
| C13 | 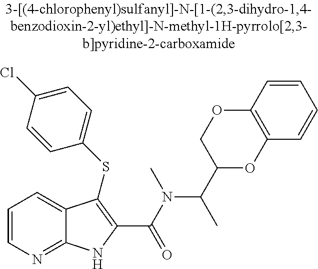 [SMILES: CC(C1COc2ccccc2O1)N(C)C(=O)c1[nH]c2ncccc2c1Sc1ccc(Cl)cc1] | 480.1144 |
| C14 | 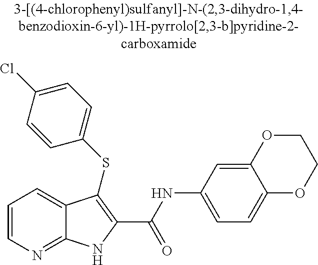 [SMILES: O=C(Nc1ccc2c(c1)OCCO2)c1[nH]c2ncccc2c1Sc1ccc(Cl)cc1] | 438.0674 |
| C15 | 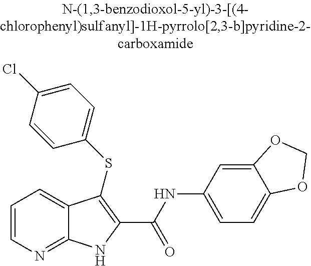 [SMILES: O=C(Nc1ccc2c(c1)OCO2)c1[nH]c2ncccc2c1Sc1ccc(Cl)cc1] | 424.0517 |
| C16 | 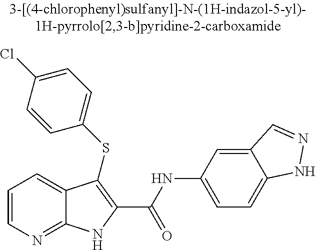 [SMILES: O=C(Nc1ccc2[nH]ncc2c1)c1[nH]c2ncccc2c1Sc1ccc(Cl)cc1] | 420.0695 |
| C17 | 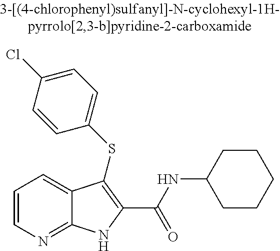 [SMILES: O=C(NC1CCCCC1)c1[nH]c2ncccc2c1Sc1ccc(Cl)cc1] | 386.1089 |
| C18 | 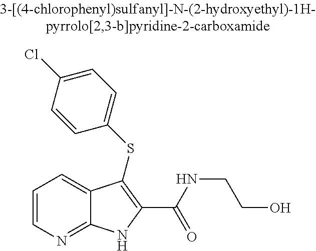 [SMILES: O=C(NCCO)c1[nH]c2ncccc2c1Sc1ccc(Cl)cc1] | 348.0564 |
| C19 | 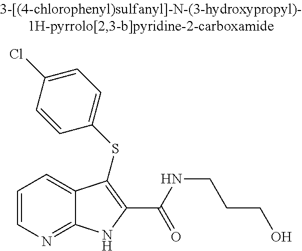 [SMILES: O=C(NCCCO)c1[nH]c2ncccc2c1Sc1ccc(Cl)cc1] | 362.0724 |
| C20 | 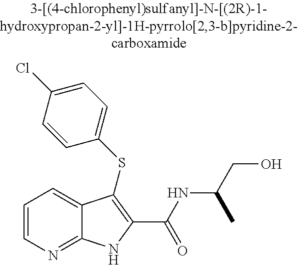 [SMILES: C[C@H](CO)NC(=O)c1[nH]c2ncccc2c1Sc1ccc(Cl)cc1] | 362.0725 |
| C21 | 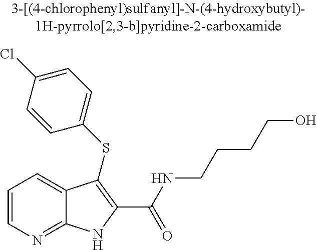 [SMILES: O=C(NCCCCO)c1[nH]c2ncccc2c1Sc1ccc(Cl)cc1] | 376.0883 |
| C22 | 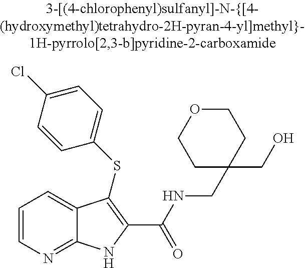 [SMILES: O=C(NCC1(CO)CCOCC1)c1[nH]c2ncccc2c1Sc1ccc(Cl)cc1] | 432.1146 |

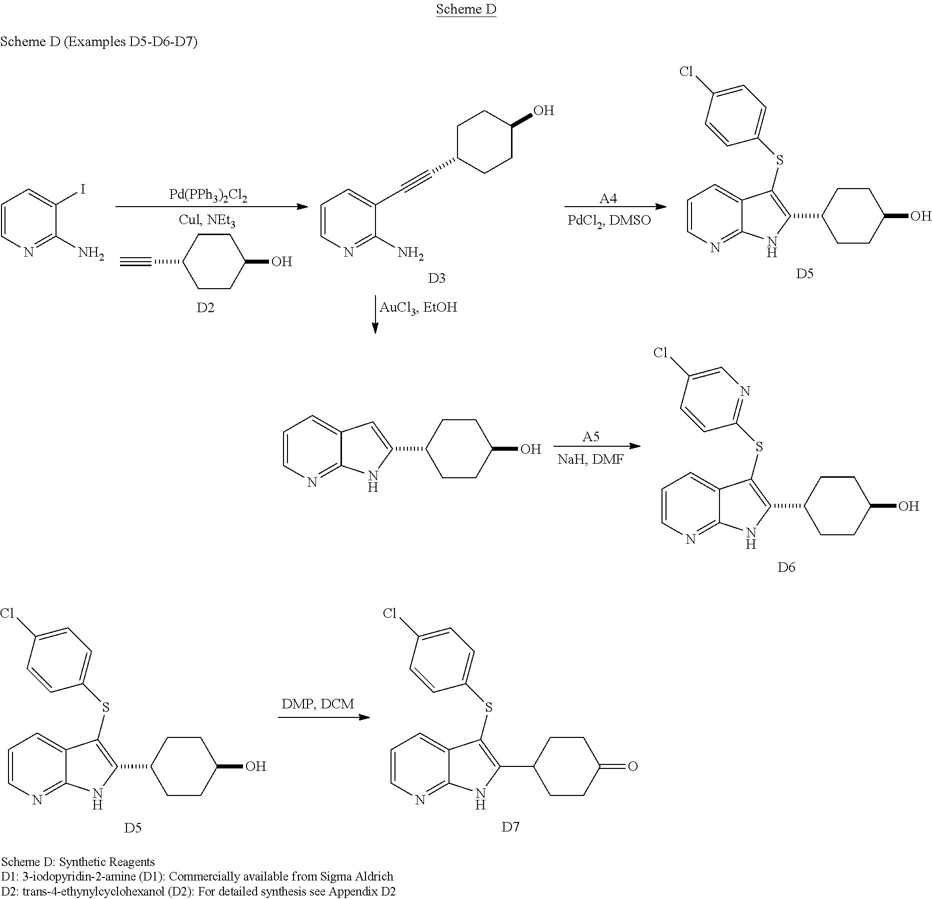

### Scheme D: Synthetic Procedures

### Step D3-1: trans-4-[(2-aminopyridin-3-yl)ethynyl]cyclohexanol (D3)

3-iodopyridin-2-amine (D1) (2 g, 9.09 mmol), trans-4-ethynylcyclohexanol (D2) (1.47 g, 11.8 mmol), CuI (87 mg, 0.455 mmol), and PdCl₂(PPh₃)₂ were stirred in anhydrous THF (36.4 ml) under a inert atmosphere. Triethylamine (3.80 mL, 27.3 mmol) was added to this solution and the reaction mixture was stirred for 6 hours. The crude reaction mixture was diluted with ethylacetate and filtered through celite. The resulting solution was concentrated under reduced pressure, and purified by normal phase chromatography (silica gel, 50-100% hexanes-EtOAc) to yield 1.30 g of a white solid. LCMS (M+1)=217.3

### Step D4-1: trans-4-(1H-pyrrolo[2,3-b]pyridin-2-yl)cyclohexanol (D4)

trans-4-[(2-aminopyridin-3-yl)ethynyl]cyclohexanol (D3) (100 mg, 0.462 mmol) was dissolved in ethanol and heated to 70′C. To this reaction mixture was added AuCl₃ (4.21 mg, 0.014 mmol) and the reaction was allowed to stir for 4 hours. The reaction mixture was then concentrated under reduced pressure, and purified by normal phase chromatography (silica gel, 50-100% hexanes-EtOAc) to yield 83 mg of a white solid. LCMS (M+1)=217.3

### Step D5-1: trans-4-{3-[(4-chlorophenyl)sulfanyl]-1H-pyrrolo[2,3-b]pyridin-2-yl}cyclohexanol (D5)

A stirring mixture of trans-4-[(2-aminopyridin-3-yl)ethynyl]cyclohexanol (D3) (100 mg, 0.462 mmol), 1,1′-disulfanediylbis(4-chlorobenzene) (A4) (133 mg, 0.462 mmol), and PdCl₂ (8.2 mg, 0.046 mmol) in DMSO was heated to 80′C under an inert atmosphere for 18 hours. The reaction mixture was then poured into ethylacetate, washed with brine, extracted and concentrated under reduced pressure. The crude reaction mixture was then purified by reverse phase chromatography (5%/95% ACN/H20 to 95%/5% ACN/H2O over 10 min). Pure fractions were placed on the lyophilizer overnight to yield a white solid. ¹H NMR (CD₃OD) δ 8.15 (d, J=4.7 Hz, 1H), 7.73 (d, J=7.8 Hz, 1H), 7.85 (d, J=7.7 Hz, 1H), 7.12 (d, J=8.5 Hz, 2H), 7.07 (dd, J=7.8 Hz, 4.9 Hz, 1H), 7.05 (d, J=8.5 Hz, 2H), 3.61 (br. m, 1H), 3.13 (br. m, 1H), 2.00 (m, 2H), 1.2 (m, 4H). LCMS (M+1)=359.1, HRMS Calculated=359.0979, Measured=359.0981

### Step D6-1: trans-4-{3-[(5-chloropyridin-2-yl)sulfanyl]-1H-pyrrolo[2,3-b]pyridin-2-yl}cyclohexanol (D6)

Starting from trans-4-(1H-pyrrolo[2,3-b]pyridin-2-yl)cyclohexanol (D4) (100 mg, 0.462 mmol) a similar experimental procedure was used as in step A8-1 with the following modification. After the reaction was complete, the reaction mixture was quenched with water and extracted with ethyl acetate. The organic layer was washed with brine and concentrated under reduced pressure. The reaction mixture was then concentrated under reduced pressure, and purified by normal phase chromatography (silica gel, 50-100% hexanes-EtOAc) to yield D6 as a white solid. ¹H NMR (MeOD) δ 8.32 (d, J=2.44 Hz, 1H), 8.22 (dd, J=4.9, 1.5 Hz, 1H), 7.79 (dd, J=7.6, 1.2 Hz, 1H), 7.50 (dd, J=8.9, 2.8 Hz, 2H), 7.12 (dd, J=7.9, 4.9 Hz, 1H), 6.66 (d, J=8.6 Hz, 1H), 3.63 (m, 1H), 3.17 (m, 1H), 2.03 (br. m, J=9.2 Hz, 2H), 1.86 (br in, 4H), 1.43-1.36 (m, 2H). LCMS (M+1)=360.1, HRMS Calculated=360.0932, Measured=360.0934

### Step D7-1: 4-{3-[(4-chlorophenyl)sulfanyl]-1H-pyrrolo[2,3-b]pyridin-2-yl}cyclohexanone (D7)

trans-4-{3-[(4-chlorophenyl)sulfanyl]-1H-pyrrolo[2,3-b]pyridin-2-yl}cyclohexanol (D5) (7 mg, 0.020 mmol), and Dess-Matrin periodinane (8.27 mg, 0.020 mmol) were dissolved in dichloromethane and the reaction mixture was allowed to for 15 mins. The reaction mixture was concentrated under reduced pressure and purified by The crude reaction mixture was then purified by reverse phase chromatography (5%/95% ACN/H20 to 95%/5% ACN/H₂O over 10 min). ¹H NMR (CDCl₃) δ 11.3 (br. s, 1H), 8.27 (d, J=4.4 Hz, 1H), 7.86 (d, J=7.9 Hz, 1H), 7.16 (m, 1H), 7.15 (d, J=8.8 Hz, 2H), 6.94 (d, J=8.8 Hz, 2H), 3.81 (m, 1H), 2.57 (br. m, 4H), 2.22 (m, 4H). LCMS (M+1)=357.3, HRMS Calculated=357.0823, Measured=357.0830

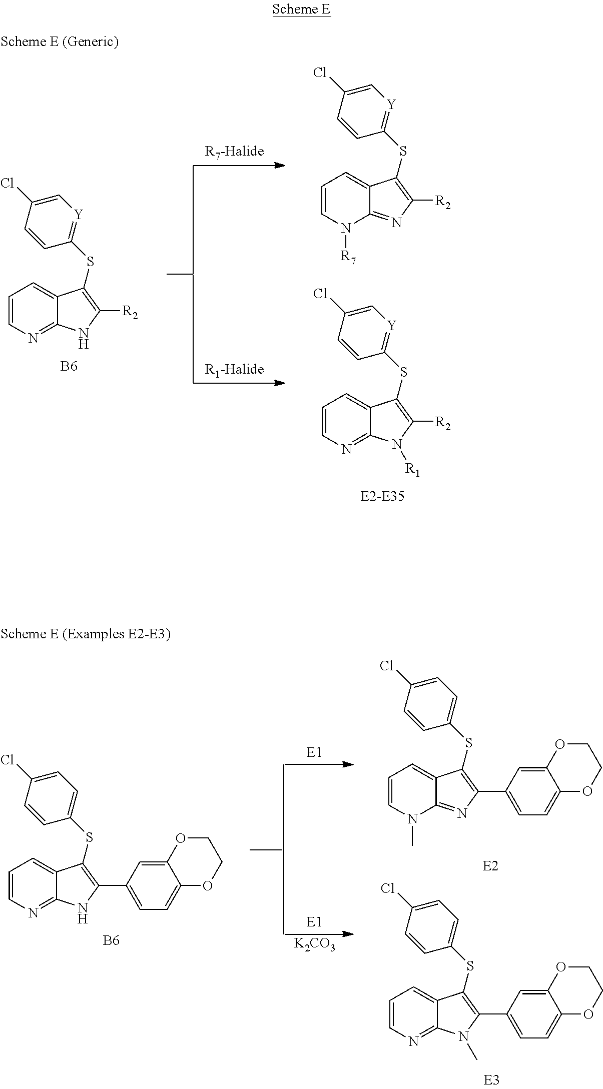

### Scheme E: Synthetic Reagents

E1: Iodomethane (E1): Commercially available from Fisher Scientific.

### Scheme E: Synthetic Procedures

### Step E2-1: 3-[(4-chlorophenyl)sulfanyl]-2-(2,3-dihydro-1,4-benzodioxin-6-yl)-7-methyl-7H-pyrrolo[2,3-b]pyridine (E2)

3-[(4-chlorophenyl)sulfanyl]-2-(2,3-dihydro-1,4-benzodioxin-6-yl)-1H-pyrrolo[2,3-b]pyridine (136) (200 mg, 0.506 mmol) was dissolved in anhydrous dimethylformamide (5.1 mL) in a sealed tube under argon atmosphere. Iodomethane (E1) (34.8 μL, 0.557 mmol) was added dropwise via syringe and the resulting solution was heated to 85° C. for 4 hours. The crude reaction mixture was than cooled to 25° C. and Hunig's base (265 μL, 1.52 mmol) was added to neutralize the pH and the resulting the solution was stirred for 10 minutes. The crude product was purified using reverse phase chromatography. The appropriate fractions were extracted into ethyl acetate and washed with saturated sodium bicarbonate and brine to yield 172 mg of a yellow solid. ¹H NMR (CDCl₃): δ 7.97 (d, J=8 Hz, 1H), 7.88 (d, J=2 Hz, 1H), 7.81 (dd, J=8 Hz, 2 Hz, 1H), 7.59 (d, J=5.7 Hz, 1H), 7.09 (d, J=8.8 Hz, 2H), 6.93 (d, J=8.8 Hz, 2H), 6.87 (m, 2H), 4.35 (s, 3H), 4.27 (s, 4H). LCMS (M+1)=409.3, HRMS Calculated=409.0772, Measured=409.0768

### Step E3-1: 3-[(4-chlorophenyl)sulfanyl]-2-(2,3-dihydro-1,4-benzodioxin-6-yl)-1-methyl-1H-pyrrolo[2,3-b]pyridine (E3)

3-[(4-chlorophenyl)sulfanyl]-2-(2,3-dihydro-1,4-benzodioxin-6-yl)-1H-pyrrolo[2,3-b]pyridine (BX) (200 mg, 0.506 mmol) and potassium carbonate (140 mg, 1.01 mmol) were dissolved in anhydrous dimethylformamide (5.1 mL) and placed under argon atmosphere. Iodomethane (E1) (34.8 μL, 0.557 mmol) was added dropwise via syringe and the resulting solution was allowed to stir at 25° C. for 16 hours. The crude reaction mixture was filtered over a pad of celite and the crude product was purified using reverse phase chromatography. The appropriate fractions were extracted into ethyl acetate and washed with saturated sodium bicarbonate and brine to yield 115 mg of a white solid. ¹H NMR (CDCl₃): δ 8.4 (d, J=4.8 Hz, 1H), 7.82 (d, J=7.7 Hz, 1H), 7.13-7.09 (m, 1H), 6.95-6.87 (m, 5H), 4.29 (s, 4H), 3.85 (s, 3H). LCMS (M+1)=409.3, HRMS Calculated=409.772, Measured=409.0768

TABLE 5
(Scheme E)

| Com- | | |
|---|---|---|
| pound | Name | |
| No. | Structure | HRMS |
| E4 | 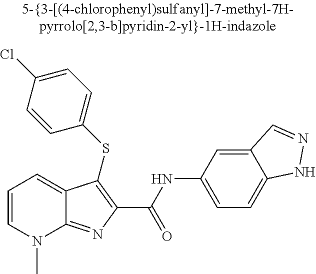 [SMILES: Cn1cccc2c(Sc3ccc(Cl)cc3)c(C(=O)Nc3ccc4[nH]ncc4c3)nc1-2] | 391.0780 |
| E5 | 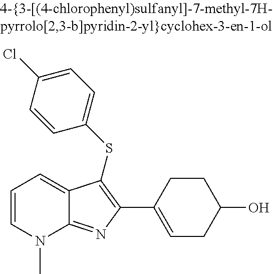 [SMILES: Cn1cccc2c(Sc3ccc(Cl)cc3)c(C3=CCC(O)CC3)nc1-2] | 371.0985 |
| E6 | 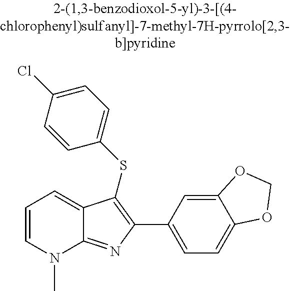 [SMILES: Cn1cccc2c(Sc3ccc(Cl)cc3)c(-c3ccc4c(c3)OCO4)nc1-2] | 395.0614 |
| E7 | 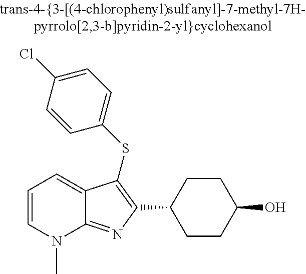 [SMILES: Cn1cccc2c(Sc3ccc(Cl)cc3)c([C@H]3CC[C@H](O)CC3)nc1-2] | 373.1138 |
| E8 | 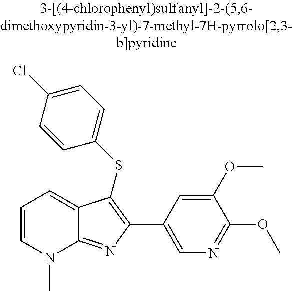 [SMILES: COc1cc(-c2nc3n(C)cccc-3c2Sc2ccc(Cl)cc2)cnc1OC] | 412.0888 |
| E9 | 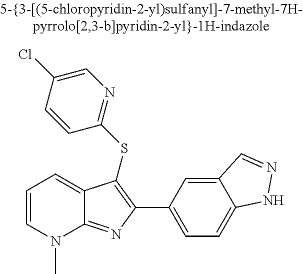 [SMILES: Cn1cccc2c(Sc3ccc(Cl)cn3)c(-c3ccc4[nH]ncc4c3)nc1-2] | 392.0738 |
| E10 | 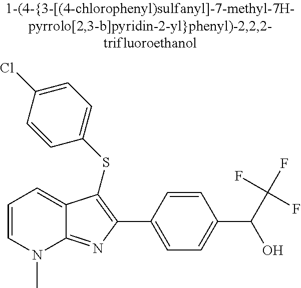 [SMILES: Cn1cccc2c(Sc3ccc(Cl)cc3)c(-c3ccc(C(O)C(F)(F)F)cc3)nc1-2] | 449.0706 |
| E11 | 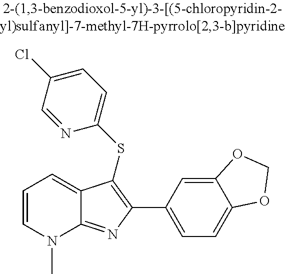 [SMILES: Cn1cccc2c(Sc3ccc(Cl)cn3)c(-c3ccc4c(c3)OCO4)nc1-2] | 396.0572 |
| E12 | 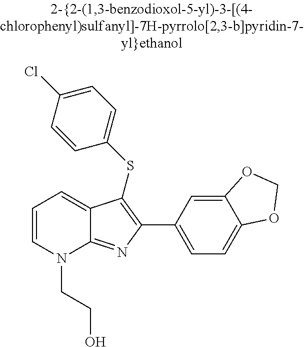 [SMILES: OCCn1cccc2c(Sc3ccc(Cl)cc3)c(-c3ccc4c(c3)OCO4)nc1-2] | 425.0726 |
| E13 | 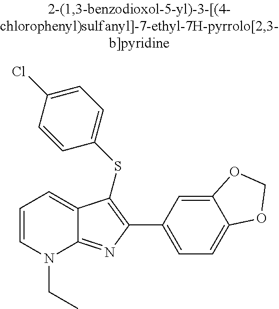 [SMILES: CCn1cccc2c(Sc3ccc(Cl)cc3)c(-c3ccc4c(c3)OCO4)nc1-2] | 409.0776 |
| E14 | 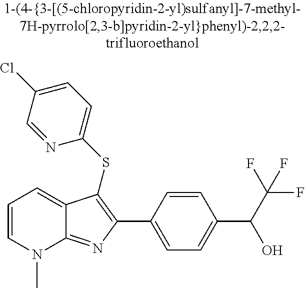 [SMILES: Cn1cccc2c(Sc3ccc(Cl)cn3)c(-c3ccc(C(O)C(F)(F)F)cc3)nc1-2] | 450.0660 |
| E15 | 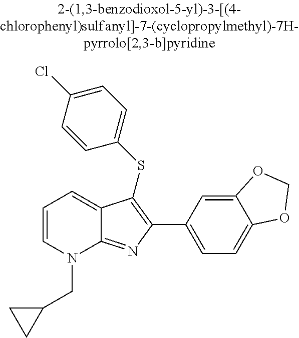 [SMILES: Clc1ccc(Sc2c3cccn(CC4CC4)c-3nc2-c2ccc3c(c2)OCO3)cc1] | 435.0934 |
| E16 | 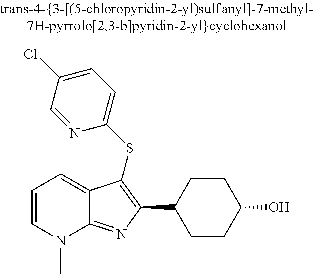 | 374.1095 |
| E17 | 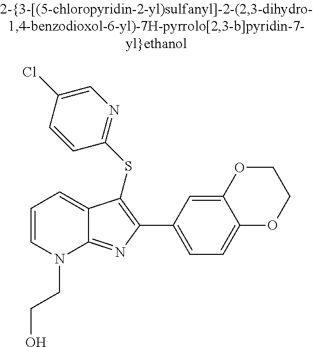 [SMILES: OCCn1cccc2c(Sc3ccc(Cl)cn3)c(-c3ccc4c(c3)OCCO4)nc1-2] | 440.0832 |
| E18 | 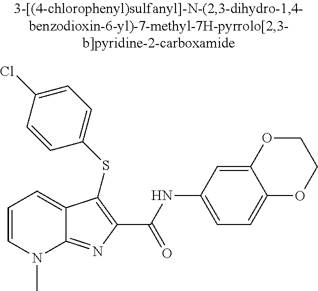 [SMILES: Cn1cccc2c(Sc3ccc(Cl)cc3)c(C(=O)Nc3ccc4c(c3)OCCO4)nc1-2] | 452.0839 |
| E19 | 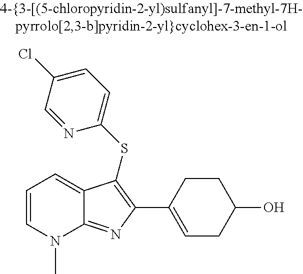 [SMILES: Cn1cccc2c(Sc3ccc(Cl)cn3)c(C3=CCC(O)CC3)nc1-2] | 372.0938 |
| E20 | 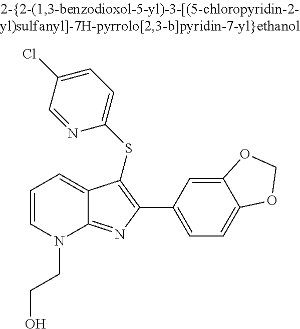 [SMILES: OCCn1cccc2c(Sc3ccc(Cl)cn3)c(-c3ccc4c(c3)OCO4)nc1-2] | 426.0677 |
| E21 | 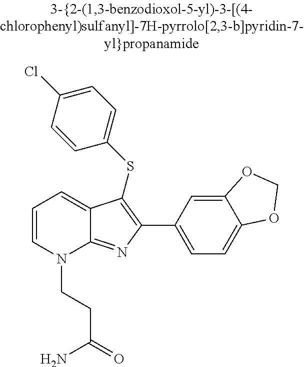 [SMILES: NC(=O)CCn1cccc2c(Sc3ccc(Cl)cc3)c(-c3ccc4c(c3)OCO4)nc1-2] | 452.0833 |
| E22 | 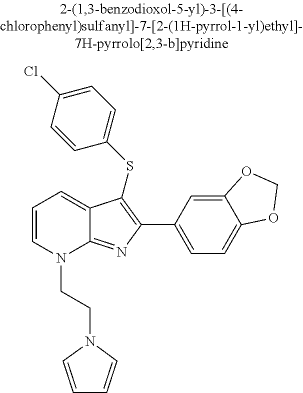 [SMILES: Clc1ccc(Sc2c3cccn(CCn4cccc4)c-3nc2-c2ccc3c(c2)OCO3)cc1] | 474.1038 |
| E23 | 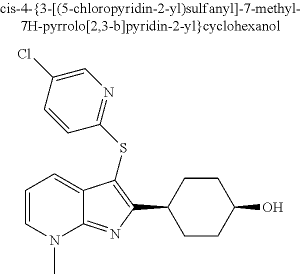 [SMILES: Cn1cccc2c(Sc3ccc(Cl)cn3)c([C@H]3CC[C@@H](O)CC3)nc1-2] | 374.1096 |
| E24 | 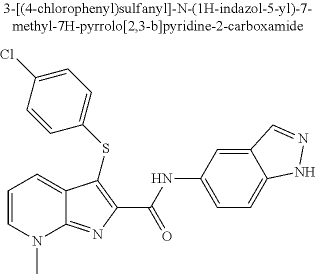 [SMILES: Cn1cccc2c(Sc3ccc(Cl)cc3)c(C(=O)Nc3ccc4[nH]ncc4c3)nc1-2] | 434.0828 |
| E25 | 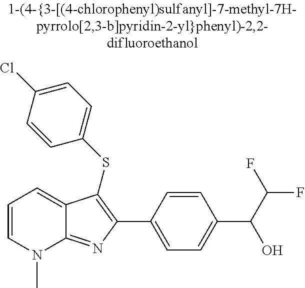 [SMILES: Cn1cccc2c(Sc3ccc(Cl)cc3)c(-c3ccc(C(O)C(F)F)cc3)nc1-2] | 431.0780 |
| E26 | 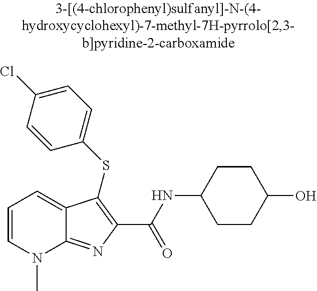 [SMILES: Cn1cccc2c(Sc3ccc(Cl)cc3)c(C(=O)NC3CCC(O)CC3)nc1-2] | 416.1186 |
| E27 | 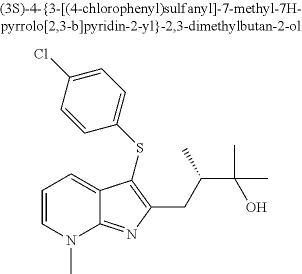 [SMILES: C[C@@H](Cc1nc2n(C)cccc-2c1Sc1ccc(Cl)cc1)C(C)(C)O] | 375.1284 |
| E28 | 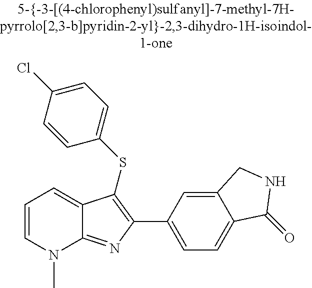 [SMILES: Cn1cccc2c(Sc3ccc(Cl)cc3)c(-c3ccc4c(c3)CNC4=O)nc1-2] | 406.0784 |
| E29 | 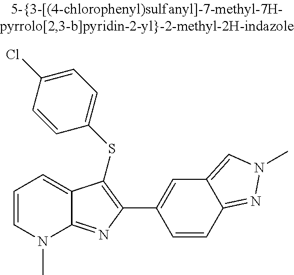 [SMILES: Cn1cc2cc(-c3nc4n(C)cccc-4c3Sc3ccc(Cl)cc3)ccc2n1] | 405.0928 |
| E30 | 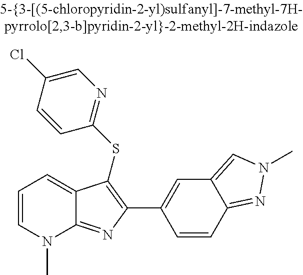 [SMILES: Cn1cc2cc(-c3nc4n(C)cccc-4c3Sc3ccc(Cl)cn3)ccc2n1] | 406.0888 |
| E31 | 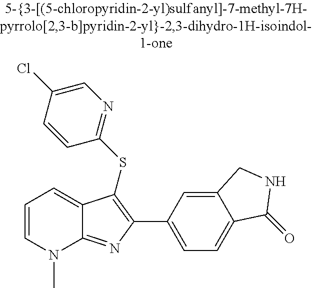 [SMILES: Cn1cccc2c(Sc3ccc(Cl)cn3)c(-c3ccc4c(c3)CNC4=O)nc1-2] | 407.0718 |
| E32 | 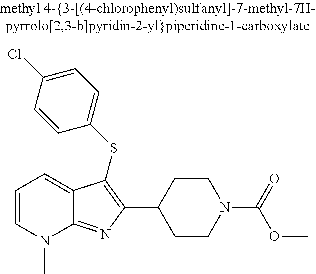 [SMILES: COC(=O)N1CCC(c2nc3n(C)cccc-3c2Sc2ccc(Cl)cc2)CC1] | 416.1186 |
| E33 | 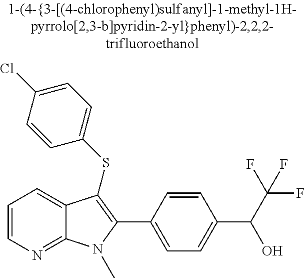 [SMILES: Cn1c(-c2ccc(C(O)C(F)(F)F)cc2)c(Sc2ccc(Cl)cc2)c2cccnc21] | 449.0707 |
| E34 | 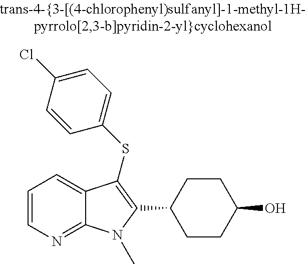 [SMILES: Cn1c2ncccc2c(Sc2ccc(Cl)cc2)c1[C@H]1CC[C@H](O)CC1] | 373.1141 |
| E35 | 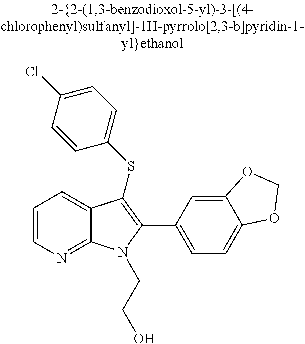 [SMILES: OCCn1c(-c2ccc3c(c2)OCO3)c(Sc2ccc(Cl)cc2)c2cccnc21] | 425.0726 |

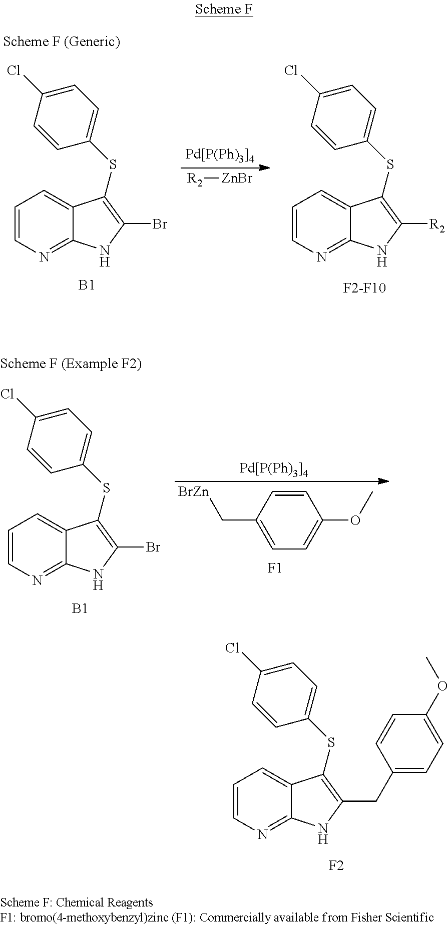

### Scheme F: Synthetic Procedures

### Step F2-1: 3-[(4-chlorophenyl)sulfanyl]-2-(4-methoxybenzyl)-1H-pyrrolo[2,3-b]pyridine (F2)

2-bromo-3-[(4-chlorophenyl)sulfanyl]-1H-pyrrolo[2,3-b]pyridine (B1) (30.0 mg, 0.088 mmol) was dissolved in degassed dioxane (0.90 mL) and placed under argon atmosphere. Tetrakis(triphenylphosphine)palladium (10.2 mg, 8.8 μmole) was added in one portion as a solid to the solution. The resulting solution was heated to 100° C. for 0.5 hours using microwave irradiation. The crude reaction mixture was filtered over celite, diluted with ethyl acetate, and washed with brine. The organics were dried over sodium sulfate and concentrated in vacuo. The crude product was purified using reverse phase chromatography. The appropriate fractions were extracted into ethyl acetate and washed with saturated sodium bicarbonate and brine to yield 24 mg of a clear oil. ¹H NMR (CDCl₃): δ 8.11 (d, J=5.1. Hz, 1H), 7.82 (d, J=8.1 Hz, 2H), 7.15-7.05 (m, 6H), 6.81 (dd, J=8.1 Hz, 5.1 Hz, 1H), 4.3 (s, 2H), 3.75 (s, 3H). LCMS (M+1)=381.3, HRMS Calculated=381.0823, Measured=381.0830

TABLE 6
(Scheme F)

| Com- | | |
|---|---|---|
| pound | Name | |
| No. | Structure | HRMS |
| F3 | 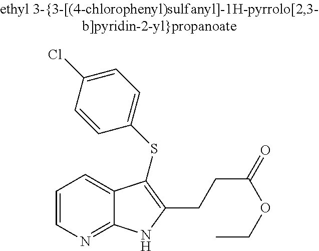 [SMILES: CCOC(=O)CCc1[nH]c2ncccc2c1Sc1ccc(Cl)cc1] | 361.0769 |
| F4 | 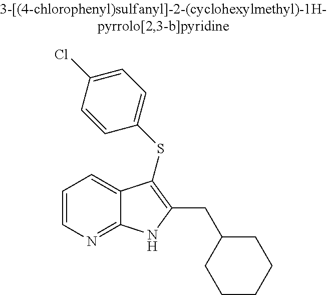 [SMILES: Clc1ccc(Sc2c(CC3CCCCC3)[nH]c3ncccc23)cc1] | 357.1183 |
| F5 | 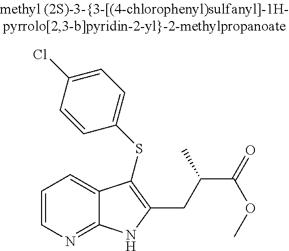 [SMILES: COC(=O)[C@@H](C)Cc1[nH]c2ncccc2c1Sc1ccc(Cl)cc1] | 361.0768 |
| F6 | 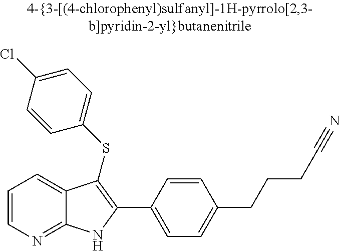 [SMILES: N#CCCCc1ccc(-c2[nH]c3ncccc3c2Sc2ccc(Cl)cc2)cc1] | 328.0665 |
| F7 | 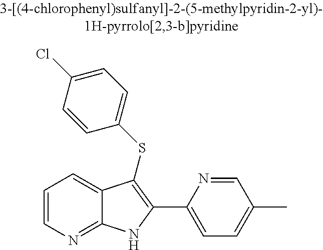 [SMILES: Cc1ccc(-c2[nH]c3ncccc3c2Sc2ccc(Cl)cc2)nc1] | 352.3⁹ |
| F8 | 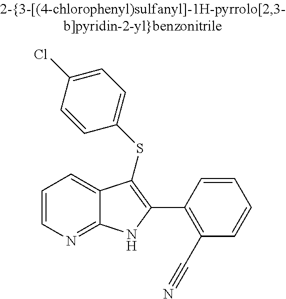 [SMILES: N#Cc1ccccc1-c1[nH]c2ncccc2c1Sc1ccc(Cl)cc1] | 362.0522 |
| F9 | 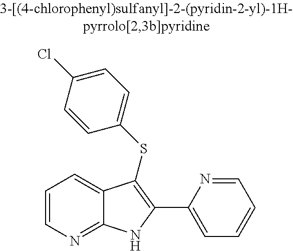 [SMILES: Clc1ccc(Sc2c(-c3ccccn3)[nH]c3ncccc23)cc1] | 338.0518 |
| F10 | 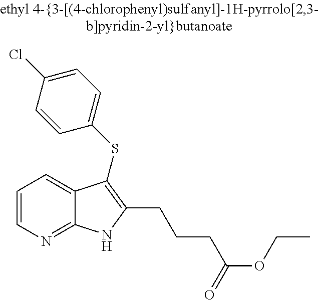 [SMILES: CCOC(=O)CCCc1[nH]c2ncccc2c1Sc1ccc(Cl)cc1] | 375.0922 |

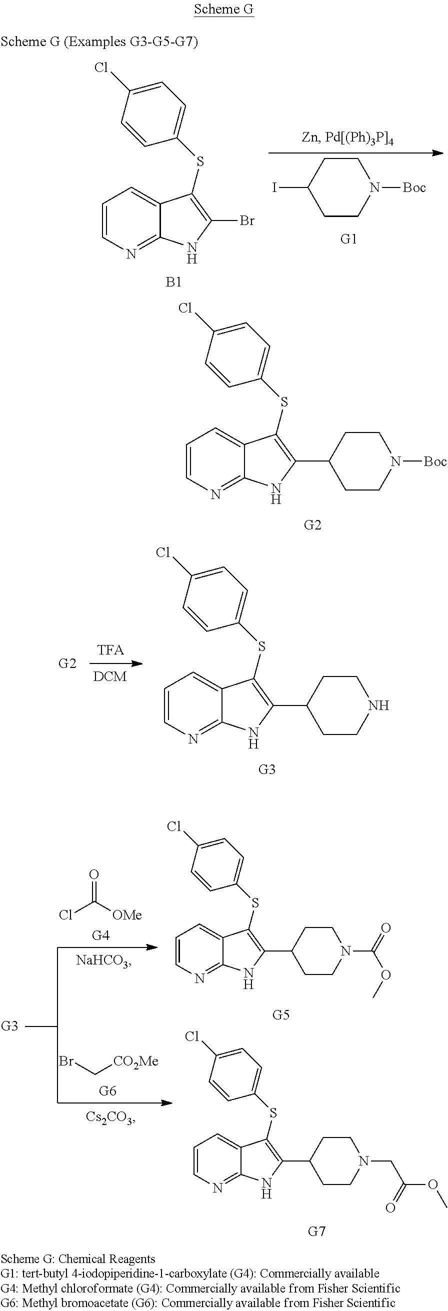

### Scheme G: Synthetic Procedures

### Step G2: tert-butyl 4-{3-[(4-chlorophenyl)sulfanyl]-1H-pyrrolo[2,3-b]pyridin-2-yl}piperidine-1-carboxylate (G2)

tert-butyl 4-iodopiperidine-1-carboxylate (G1)(710 mg, 2.82 mmol) was dissolved in degassed THF (4.1 mL) and placed under argon atmosphere. An activated zinc solution (3.0 mL, 2.82 mmol, 0.75 M solution) was added dropwise to the stirring solution and the resulting mixture was stirred at 25° C. for 2 hours. The resulting zincate solution was then added dropwise via syringe to a solution of 2-bromo-3-[(4-chlorophenyl)sulfanyl]-1H-pyrrolo[2,3-b]pyridine (B1)(310 mg, 0.913 mmol) and bis(tri-t-butylphosphine)palladium (46.6 mg, 0.091 mmol) in degassed THF (5.0 mL) under argon atmosphere. The resulting solution was heated to 100° C. for 1 hour using microwave irradiation. The crude reaction mixture was then filtered over celite, diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 202 mg of a yellow oil. ¹H NMR (CDCl₃): δ 8.33 (d, J=4.9 Hz, 1H), 7.85 (d, J=7.9 Hz, 1H), 7.12 (d, J=6.8 Hz, 2H), 7.10 (dd, J=7.9 Hz, 4.9 Hz, 1H), 6.92 (d, J=6.8 Hz, 2H), 3.45 (br m, 1H), 2.85 (br m, 2H), 2.15 (br m, 2H), 1.62 (br m, 1H), 1.51 (s, 9H), 1.24 (br m, 1H). LCMS (M+1)=−444.4.

### Step G3: 3-[(4-chlorophenyl)sulfanyl]-2-(piperidin-4-yl)-1H-pyrrolo[2,3-b]pyridine (G3)

tert-butyl 4-{3-[(4-chlorophenyl)sulfanyl]-1H-pyrrolo[2,3-b]pyridin-2-yl}piperidine-1-carboxylate (35 mg, 0.079 mmol) was dissolved in methylene chloride (1.0 mL) and trifluoroacetic acid (30.4 μL, 0.394 mmol) was dropwise via syringe. The resulting solution was allowed to stir at 25° C. for 1 hour. The solution was then concentrated in vacuo and the crude product was purified using reverse phase chromatography. The appropriate fractions were extracted into ethyl acetate and washed with saturated sodium bicarbonate and brine to yield 20 mg of a colorless oil. ¹H NMR (CDCl₃): δ 8.33 (d, J=4.9 Hz, 1H), 7.85 (d, J=7.9 Hz, 1H), 7.12 (d, J=6.8 Hz, 2H), 7.10 (dd, J=7.9 Hz, 4.9 Hz, 1H), 6.92 (d, J=6.8 Hz, 2H), 3.45 (br m, 1H), 2.85 (br m, 2H), 2.15 (br m, 2H), 1.62 (br in, 1H), 1.24 (br in, 1H) LCMS (M+1)=344.0, HRMS Calculated=344.0983, Measured=344.0984

### Step G5: Methyl 4-{3-[(4-chlorophenyl)sulfanyl]-1H-pyrrolo[2,3-b]pyridin-2-yl}piperidine-1-carboxylate (G5)

3-[(4-chlorophenyl)sulfanyl]-2-(piperidin-4-yl)-1H-pyrrolo[2,3-b]pyridine (G3) (28 mg, 0.081 mmol) was dissolved in 2:1 solution of chloroform and aqueous saturated sodium bicarbonate (1.0 mL). Methyl chloroformate (G4) (6.31 μL, 0.081 mmol) was added dropwise via syringe and the resulting solution was allowed to stir at 25° C. for 1 hour. The solution was then partitioned between chloroform and water, the combined organics were dried using sodium sulfate and concentrated in vacuo. The crude product was purified using reverse phase chromatography. The appropriate fractions were extracted into ethyl acetate and washed with saturated sodium bicarbonate and brine to yield 26 mg of a colorless oil. ¹H NMR (CDCl₃): δ 8.22 (d, J=5.1 Hz, 1H), 7.81 (d, J=7.9 Hz, 1H), 7.10 (d, J=6.8 Hz, 2H), 7.05 (dd, J=7.9 Hz, 5.1 Hz, 1H), 6.88 (d, J=6.8 Hz, 2H), 3.72 (s, 3H), 3.43 (br m, 1H), 2.86 (br m, 2H), 1.95 (br m, 2H), 1.81 (br m, 2H), 1.65 (br m, 1H), 1.19 (br m, 1H) LCMS (M+1)=402.2, HRMS Calculated=402.1038, Measured=402.1032

### Step G7: Methyl (4-{3-[(4-chlorophenyl)sulfanyl]-1H-pyrrolo[2,3-b]pyridin-2-yl}piperidin-1-yl)acetate (G7)

3-[(4-chlorophenyl)sulfanyl]-2-(piperidin-4-yl)-1H-pyrrolo[2,3-b]pyridine (G3) (35 mg, 0.102 mmol) and cesium carbonate (99 mg, 0.305 mmol) were dissolved in anhydrous DMF (1.0 mL). Methyl bromoacetate (9.4 μL, 0.102 mmol) was added dropwise to the stirring solution and the resulting solution was allowed to stir at 25° C. for 1 hour. The solution was diluted with ethyl acetate and washed with aqueous lithium chloride. The organics were dried with sodium sulfate and concentrated in vacuo. The crude product was purified using reverse phase chromatography. The appropriate fractions were extracted into ethyl acetate and washed with saturated sodium bicarbonate and brine to yield 35 mg of a colorless oil. ¹H NMR (CDCl₃): □ 8.72 (d, J=5.0 Hz, 1H), 7.82 (d, J=7.9 Hz, 1H), 7.18 (dd, J=7.9 Hz, 5.0 Hz, 1H), 7.10 (d, J=6.9 Hz, 2H), 6.91 (d, J=6.9 Hz, 2H), 3.74 (s, 3H), 3.30 (s, 3H), 3.05 (d, 11 Hz, 2H), 2.37 (t, J=11 Hz, 2H), 2.20 (q, J=14 Hz, 2H), 1.85 (d, J=14 Hz, 2H). LCMS (M+1)=416.3, HRMS Calculated=416.1194, Measured=416.1189

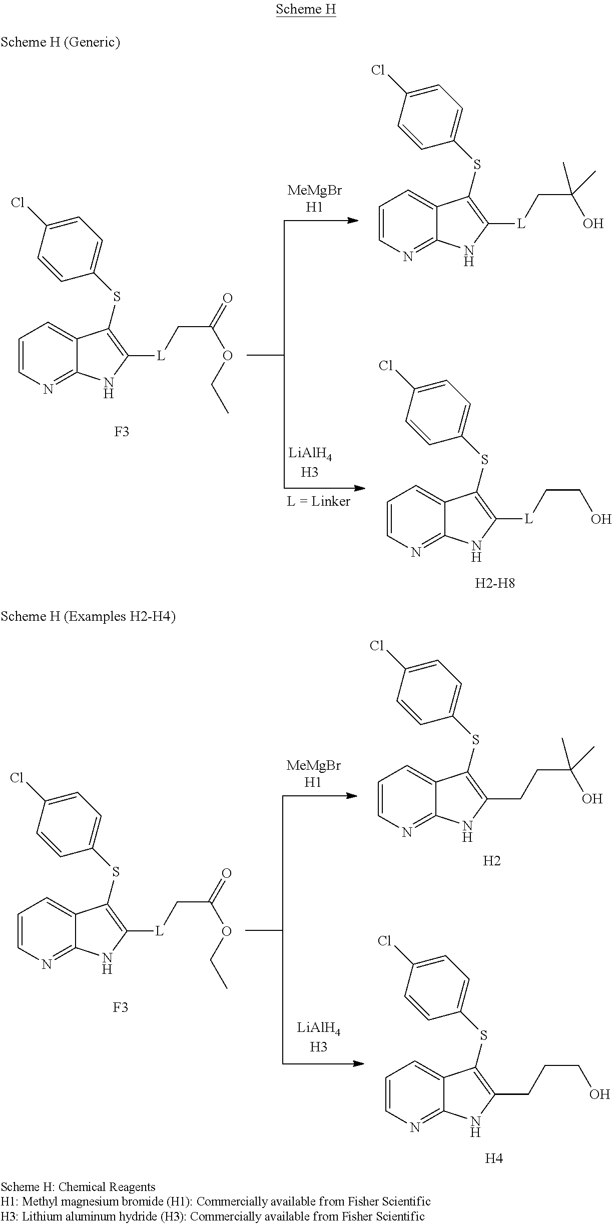

### Scheme H: Synthetic Procedures

### Step H2: 4-{3-[(4-chlorophenyl)sulfanyl]-1H-pyrrolo[2,3-b]pyridin-2-yl}-2-methylbutan-2-ol (H2)

Ethyl 3-{3-[(4-chlorophenyl)sulfanyl]-1H-pyrrolo[2,3-b]pyridin-2-yl}propanoate (F3)(25 mg, 0.069 mmol) was dissolved in anhydrous THF (800 μL), placed under argon atmosphere and cooled to 0° C. Methyl magnesium bromide (92 μL, 0.28 mmol, 3 M solution) was added dropwise to the stirring solution and the resulting mixture was stirred at 0° C. for 1 hour. The reaction mixture was quenched with aqueous ammonium chloride, diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 18 mg of a colorless oil. ¹H NMR (CDCl₃): δ 8.39 (d, J=4.9 Hz, 1H), 7.81 (d, J=7.8 Hz, 1H), 7.12 (d, J=6.8 Hz, 2H), 7.08 (dd, J=7.8 Hz, 4.9 Hz, 1H), 6.93 (d, 6.8 Hz, 2H), 3.12 (t, J=7.8 Hz, 2H), 1.89 (t, J=7.8 Hz, 2H), 1.75-1.60 (br s, 1H), 1.31 (s, 6H). LCMS (M+1)=347.2, HRMS Calculated=347.0979, Measured=347.0973

### Step H4: 3-{3-[(4-chlorophenyl)sulfanyl]-1,1-pyrrolo[2,3-b]pyridin-2-yl}propan-1-ol (H4)

Ethyl 3-{3-[(4-chlorophenyl)sulfanyl]-1H-pyrrolo[2,3-b]pyridin-2-yl}propanoate (F3)(25 mg, 0.069 mmol) was dissolved in anhydrous THF (800 μL), placed under argon atmosphere and cooled to 0° C. Lithium aluminum hydride (138 μL, 0.14 mmol, 1 M solution) was added dropwise to the stirring solution and the resulting mixture was stirred at 0° C. for 0.5 hours. The reaction mixture was quenched with aqueous sodium potassium tartrate and stirred for 3 hours. The resulting solution was diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 13 mg of a white solid. ¹H NMR (CDCl₃): δ 8.31 (dd, J=4.8 Hz, 1.5 Hz, 1H), 7.81 (dd, J=7.7 Hz, 1.5 Hz, 1H), 7.12 (d, J=8.6 Hz, 2H), 7.10 (dd, J=7.7 Hz, 4.8 Hz, 1H), 6.92 (d, J=8.6 Hz, 2H), 3.77 (t, J=5.8 Hz, 2H), 3.14 (t, J=6.1 Hz, 2H), 2.0 (m, 2H), 1.75-1.60 s, 1H). LCMS (M+1)=319.2, HRMS Calculated=319.0666, Measured=319.0663

TABLE 6
(Scheme H)

| Compound | Name | |
|---|---|---|
| No. | Structure | HRMS |
| H5 | 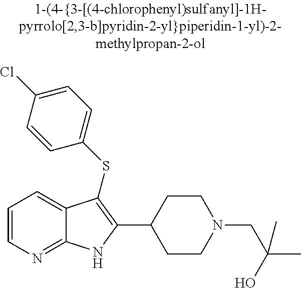 [SMILES: CC(C)(O)CN1CCC(c2[nH]c3ncccc3c2Sc2ccc(Cl)cc2)CC1] | 416.1551 |
| H6 | 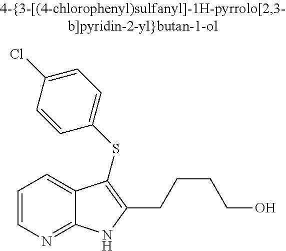 [SMILES: OCCCCc1[nH]c2ncccc2c1Sc1ccc(Cl)cc1] | 333.0819 |
| H7 | 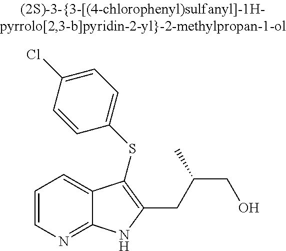 [SMILES: C[C@H](CO)Cc1[nH]c2ncccc2c1Sc1ccc(Cl)cc1] | 333.0819 |
| H8 | 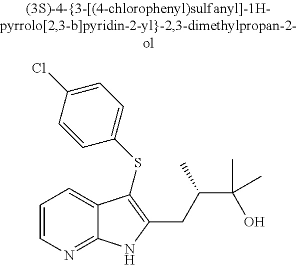 [SMILES: C[C@@H](Cc1[nH]c2ncccc2c1Sc1ccc(Cl)cc1)C(C)(C)O] | 361.1132 |

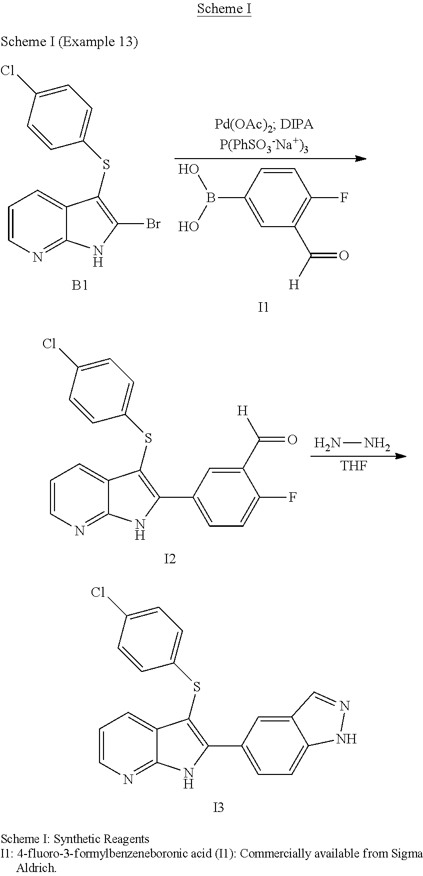

### Scheme I: Synthetic Procedures

Step I2-1: 5-{3-[(4-chlorophenyl)sulfanyl]-1H-pyrrolo[2,3-b]pyridin-2-yl}-2-fluorobenzaldehyde (I2)

2-bromo-3-[(4-chlorophenyl)sulfanyl]-1H-pyrrolo[2,3-b]pyridine, B1 (100 mg, 0.30 mmol) and 4-fluoro-3-formylbenzeneboronic acid (I1) was added to a pressure vial. A previously degassed solution of DMF (1.8 mL) and H₂O (0.470 mL) was then added and the reaction mixture was then placed under N₂ atmosphere. Triphenylphosphine-3,3′,3″-trisulfonic acid trisodium salt hydrate (113 mg, 0.18 mmol), diisopropylamine (0.74 mmol, 105 μL), and palladium(II) acetate (0.059 mmol, 13 mg) were added and the entire reaction mixture was degassed with N₂, capped and heated to 80° for 1 hour. The reaction mixture was cooled and filtered through a syringe filter. EtOAc and saturated. NaHCO₃ were added to the filtrate and the layers were separated. The aqueous layer was back extracted with EtOAc (3×) until no product is seen in aqueous layer. The organic layers were combined, washed with brine, dried over sodium sulfate, filtered and concentrated to yield a tan oil which was purified by silica gel chromatography (0% to 50% EtOAc/Hex over 30 minutes) to yield 65 mg of a white solid. LCMS (M+1)=383.3

### Step I3-1: 5-{3-[(4-chlorophenyl)sulfanyl]-1H-pyrrolo[2,3-b]pyridin-2-yl}-1H-indazole (I3)

5-{3-[(4-chlorophenyl)sulfanyl]-1H-pyrrolo[2,3-b]pyridin-2-yl}-2-fluorobenzaldehyde, I2 (60 mg, 0.157) was added to a solution of THF (1.0 mL) and hydrazine (50.2 mmol, 1.6 mL). The reaction mixture was heated to 100° for 16 hours. The hydrazine was then removed in vacuo to yield a white solid which was taken up in DCM and stirred. The mixture was filtered and the solids washed with DCM, dried and collected to yield 55 mg of desired product. ¹H NMR (DMSO) δ 8.32 (d, J=4.7 Hz, 1H), 8.24 (s, 1H), 8.19 (s, 1H), 7.83 (d, J=8.6 Hz, 1H), 7.76 (d, J=7.8 Hz, 1H), 7.64 (d, J=8.8 Hz. 1H), 7.3 (d, J=8.6 Hz, 2H), 7.15 (dd, J=7.8 Hz, J=4.7 hz, 1H), 7.01 (d, J=8.6 Hz, 2H), LCMS (M+1)=383.3, HRMS Calculated=371.0622, Measured=371.0622

### Aza-Indole CMI

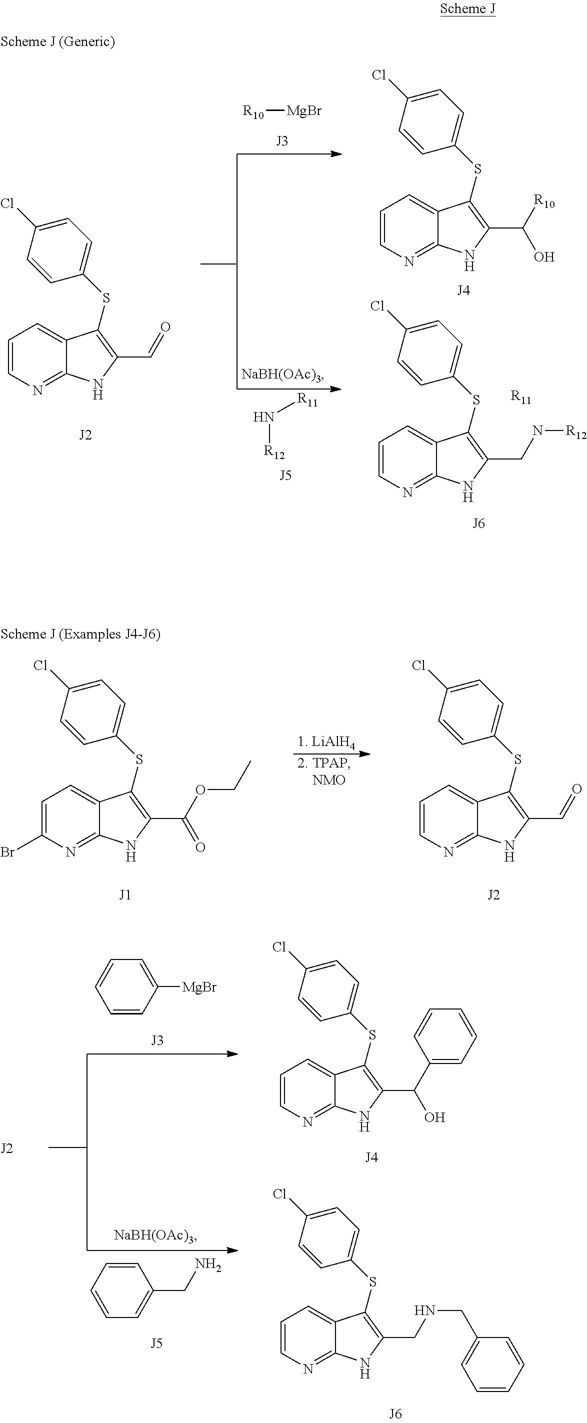

### Scheme J: Chemical Reagents

J1: Ethyl -bromo-3-[(4-cblorophexyl)sulfanyl]-1H-pyrrolo[2,3-b]pyridine-2-carboxylate (J1): See Appendix 2.
J3: Phenyl magnesium bromide (J3) Commercially available from Fisher Scientific
J5: Benzyl amine (J5) Commercially available from Fisher Scientific

### Scheme J: Synthetic Procedures

### Step J2: 3-[(4-chlorophenyl)sulfanyl]-1H-pyrrolo[2,3-b]pyridine-2-carbaldehyde (J2)

Ethyl 6-bromo-3-[(4-chlorophenyl)sulfanyl]-1H-pyrrolo[2,3-b]pyridine-2-carboxylate (1.7 g, 4.13 mmol) was dissolved in anhydrous THF (42 mL) and placed under argon atmosphere. Lithium aluminum hydride (12.4 mL, 24.8 mmol, 2 M solution) was added dropwise to the stirring solution and the resulting solution was heated to reflux for 16 hours. The reaction mixture was then cooled to 0° C. and quenched with aqueous sodium potassium tartrate and stirred for 3 hours. The resulting solution was diluted with ethyl acetate, washed with brine, dried over sodium sulfate, and concentrated in vacuo to afford 888 mg of {3-[(4-chlorophenyl)sulfanyl]-1H-pyrrolo[2,3-b]pyridin-2-yl}methanol as a white solid. {3-[(4-chlorophenyl)sulfanyl]-1H-pyrrolo[2,3-b]pyridin-2-yl}methanol (888 mg, 3.05 mmol), 4-methylmorpholine N-oxide (465 mg, 3.97 mmol), and 4 Å sieves (600 mg) were dissolved in anhydrous methylene chloride (30 mL) and placed under argon atmosphere. Tetrapropylammonium perruthenate (107 mg, 0.305 mmol) was added portionwise as a solid to the stirring solution, the resulting solution was then stirred at 25° C. for 16 hours. The crude reaction mixture was filtered over a celite pad and concentrated in vacuo. The crude product was purified using silica gel chromatography (300 g, using 15-75% ethyl acetate in hexane gradient) to afford 654 mg of the desired aldehyde as a colorless oil. LCMS (M+1)=289.2

### Step J4: {3-[(4-chlorophenyl)sulfanyl]-1H-pyrrolo[2,3-b]pyridin-2-yl}(phenyl)methanol (J4)

3-[(4-chlorophenyl)sulfanyl]-1H-pyrrolo[2,3-b]pyridine-2-carbaldehyde (32) (35 mg, 0.121 mmol) was dissolved in anhydrous THF (1.2 mL) and cooled to 0° C. under argon atmosphere. Phenyl magnesium bromide (303 μL, 0.303 mmol, 1 M solution) was added dropwise via syringe. The resulting solution was allowed to stir at 0° C. for 2 hours. The reaction mixture was quenched with aqueous ammonium chloride, diluted with ethyl acetate, washed with brine, dried over sodium sulfate, and concentrated in vacuo. The crude product was purified using reverse phase chromatography. The appropriate fractions were extracted into ethyl acetate and washed with saturated sodium bicarbonate and brine to yield 21 mg of a yellow oil. ¹H NMR (CDCl₃): δ 8.31 (d, J=4.9 Hz, 1H), 7.82 (d, J=7.8 Hz, 1H), 7.50-7.40 (m, 5H), 7.10 (dd, J=7.9 Hz, 4.9 Hz, 1H), 7.05 (d, J=6.8 Hz, 2H), 6.82 (d, J=6.8 Hz, 2H), 6.39 (s, 1H). LCMS (M+1)=367.3, HRMS Calculated=367.0666, Measured=367.0663

### Step J6: N-benzyl-1-{3-[(4-chlorophenyl)sulfanyl]-1H-pyrrolo[2,3-b]pyridin-2-yl}methanamine (J6)

3-[(4-chlorophenyl)sulfanyl]-1H-pyrrolo[2,3-b]pyridine-2-carbaldehyde (J2) (25 mg, 0.087 mmol) and benzyl amine (46.4 mg, 0.433 mmol) were dissolved in dichloroethane (1.0 mL) and placed under argon atmosphere. Sodium triacetoxyborohydride (27.5 mg, 0.130 mmol) was added portionwise as a solid and the resulting solution was allowed to stir overnight at 25° C. for 16 hours. The crude reaction mixture was then filtered over a celite pad and concentrated in vacuo. The crude product was purified using reverse phase chromatography. The appropriate fractions were extracted into ethyl acetate and washed with saturated sodium bicarbonate and brine to yield 19 mg of a colorless oil. ¹H NMR (CDCl₃): δ 8.30 (dd, J=4.8 Hz, 1.4 Hz, 1H), 7.82 (dd, J=7.8 Hz, 1.4 Hz, 1H), 7.35-7.20 (m, 5H), 7.15 (d, J=6.8 Hz, 2H), 7.05 (dd, J=7.8 Hz, 4.8 Hz, 1H), 6.88 (d, J=6.8 Hz, 2H), 4.16 (s, 2H), 3.81 (s, 2H). LCMS (M+1)=380.3, HRMS Calculated=380.0983, Measured=380.0986

TABLE 7
(Scheme J)

| Compound | Name | |
|---|---|---|
| No. | Structure | HRMS |
| J7 | 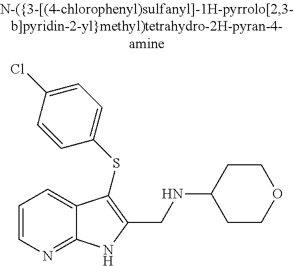 [SMILES: Clc1ccc(Sc2c(CNC3CCOCC3)[nH]c3ncccc23)cc1] | 374.1111 |
| J8 | 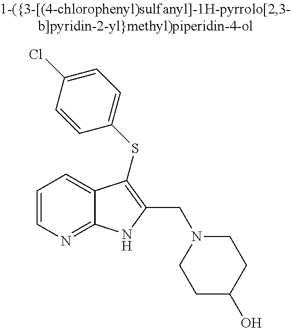 [SMILES: OC1CCN(Cc2[nH]c3ncccc3c2Sc2ccc(Cl)cc2)CC1] | 374.1088 |
| J9 | 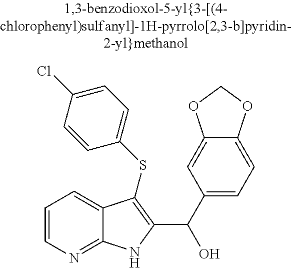 [SMILES: OC(c1ccc2c(c1)OCO2)c1[nH]c2ncccc2c1Sc1ccc(Cl)cc1] | 411.0566 |
| J10 | 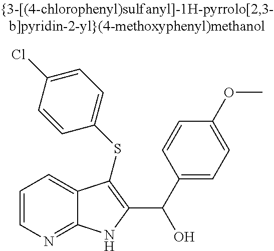 [SMILES: COc1ccc(C(O)c2[nH]c3ncccc3c2Sc2ccc(Cl)cc2)cc1] | 397.0774 |
| J11 | 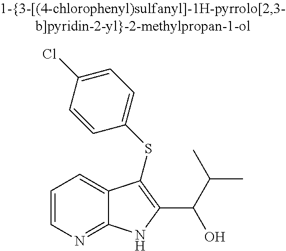 [SMILES: CC(C)C(O)c1[nH]c2ncccc2c1Sc1ccc(Cl)cc1] | 333.0824 |
| J12 | 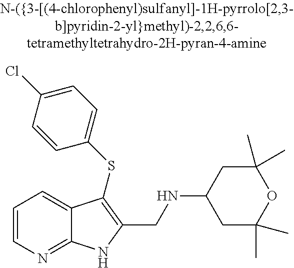 [SMILES: CC1(C)CC(NCc2[nH]c3ncccc3c2Sc2ccc(Cl)cc2)CC(C)(C)O1] | 430.1714 |

⁹LCMS data

What is claimed is:

1. A compound of the formula I: 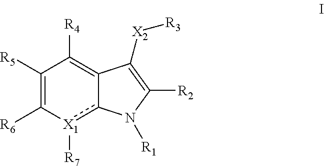
or a pharmaceutically acceptable salt thereof wherein:
n is 0, 1 or 2;
X₁ is selected from C or N;
X₂ is S or SO or SO₂;
R₁ is selected from the group consisting of:
(7) hydrogen,
(8) C₁₋₄alkyl,
(9) aryl,
(10) HET₁,
(11) (CH₂)-aryl, and
(12) (CH₂)-HET₁,
wherein choice (2), and the aryl or HET₁ of choices (3), (4), (5) and (6) are optionally mono or di-substituted with substituents selected from hydroxyl, halo, CF₃ and OCH₃;
R₂ is selected from the group consisting of
(1) hydrogen,
(2) aryl,
(3) HET₂,
(4) (CH₂)-aryl,
(5) (CH₂)-HET₂,
(6) —C₁₋₆alkyl,
(7) —C₂₋₆alkenyl,
(8) —C₃₋₆cycloalkyl,
(9) —CH₂—C₃₋₆cycloalkyl,
(10) —C₃₋₆cycloalkenyl,
(11) —NH—(CH₂)-aryl,
(12) —CH₂NH—R₁₉R₂₀,
(13) —NH—C₃₋₇cycloalkyl,
(14) —NH—C(O)R₈,
wherein R₈ is selected from the group consisting of
(a) aryl,
(b) HET₃,
(c) (CH₂)-aryl,
(d) (CH₂)-HET₃,
(e) —C₁₋₆alkyl,
(f) —C₃₋₇cycloalkyl,
(15) —C(O)NR₉R₁₀,
wherein R₉ and R₁₀ are each independently selected from the group consisting of
(a) hydrogen,
(b) hydroxyl,
(c) aryl,
(d) HET₄,
(e) —C₃₋₆cycloalkyl, optionally substituted with 1 to 4 methyl groups,
(f) —OC₃₋₆cycloalkyl,
(g) —C₁₋₄alkyl, optionally mono or di-substituted with hydroxyl, HET₅, or C₃₋₆cycloalkyl,
(h) —OC₁₋₄alkyl,
(i) —C(O)CH₃,
(j) mono, di or tri-halo C₁₋₄alkyl, and
(k) mono, di or tri-halo —OC₁₋₄alkyl,
or
R₉ and R₁₀ are joined together to form a ring with the atoms to which they are attached there is formed a heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, hydroxyC₁₋₄alkyl, haloC₁₋₄alkyl, —S(O)nC₁₋₄alkyl, and C(O)—NRaRb, wherein Ra and Rb are each independently selected from hydrogen and methyl,
wherein R₂ choices (2), (3), (4), (5), (6), (7), (8), (9), (10), (11) and (13) are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
wherein R₁₁, R₁₂, R₁₃, R₁₄, R₁₅, R₁₆, R₁₇, R₁₈, R₁₉, are each independently selected from H and C₁₋₄alkyl, optionally substituted with hydroxyl, and
R₂₀ is selected from H and C₁₋₄alkyl optionally substituted with aryl, HET₆, optionally substituted with hydroxyl or 1-4 methyl groups,
or R₁₁ and R₁₂ or R₁₃ and R₁₄ or R₁₉ and R₂₀ can be joined together to form a ring with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl and —S(O)nC₁₋₄alkyl;
R₃ is selected from the group consisting of:
(1) aryl,
(2) HET₇,
(3) —C₁₋₆alkyl,
(4) —C₃₋₆cycloalkyl, and
(5) mono, di or tri-halo C₃₋₆cycloalkyl,
wherein choices (1), (2) and (3) are each optionally mono or di-substituted with substituents independently selected from the group consisting of:
(a) hydroxy,
(b) halo,
(c) —CF₃,
(d) —OCF₃,
(e) methyl, and
(f) methoxy;
R₄, R₅ and R₆ are each independently selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₆cycloalkyl;
wherein choice (7), and the aryl or HET₅ of choices (3), (4), (5) and (6) are optionally mono or di-substituted with substituents selected from hydroxyl, halo, CF₃ and OCH₃;
R₇ is selected from the group consisting of:
(1) hydrogen,
(2) halogen,
(3) HET₈, and
(4) —C₁₋₆alkyl,
wherein choices (3) and (4) are each optionally mono or di-substituted with substituents selected from hydroxyl, C₃₋₆cycloalkyl, —C(O)—NH₂, phenyl and HET₉,
with the proviso that R₇ is other than halogen when X₁ is N.

2. A compound according to claim 1 wherein:
X₁ is N.

3. A compound according to claim 1 wherein:
X₂ is S.

4. A compound according to claim 1 wherein:
R₁ is selected from the group consisting of:
(1) hydrogen, and
(2) C₁₋₄alkyl,
wherein choice (2), is optionally mono or di-substituted with substituents selected from hydroxyl, halo, CF₃ and OCH₃.

5. A compound according to claim 1 wherein:
R₂ is selected from the group consisting of:
(1) hydrogen,
(2) aryl,
(3) (CH₂)-aryl,
(4) (CF₁₂)-HET₂,
(5) —C₁₋₆alkyl,
(6) —C₃₋₆cycloalkyl,
(7) —CH₂—C₃₋₆cycloalkyl,
(8) —C₃₋₆cycloalkenyl,
(9) —CH₂—NH—R₁₉R₂₀,
(10) —NH—C₃₋₆cycloalkyl, and
(11) —C(O)NR₉R₁₀,
wherein R₉ and R₁₀ are each independently selected from the group consisting of
(a) hydrogen,
(c) aryl,
(d) HET₄,
(e) —C₃₋₆cycloalkyl, optionally substituted with 1 to 4 methyl groups,
(f) —OC₃₋₆cycloalkyl,
(g) —C₁₋₄alkyl, optionally mono or di-substituted with hydroxyl, HET₅, or C₃₋₆cycloalkyl, and
(h) —OC₁₋₄alkyl,
or
R₉ and R₁₀ are joined together to form a ring with the atoms to which they are attached there is formed a heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, halo C₁₋₄alkyl, —C(O)—C₁₋₄alkyl, —S(O)ₙC₁₋₄alkyl, and C(O)—NRaRb, wherein Ra and Rb are each independently selected from hydrogen and methyl,
wherein R₂ choices (2), (3), (4), (5), (6), (7), (8), (9) and (10) are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(i) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R_{19g},
(s) hydroxyl, and
(t) oxo,
wherein R₁₁, R₁₂, R₁₃, R₁₄, R₁₅, R₁₆, R₁₇, R₁₈, R₁₉, are each independently selected from H and C₁₋₄alkyl, optionally substituted with hydroxyl, and
R₂₀ is selected from H and C₁₋₄alkyl optionally substituted with aryl, HET₆, optionally substituted with hydroxyl or 1-4 methyl groups,
or R₁₁ and R₁₂ or R₁₃ and R₁₄ or R₁₉ and R₂₀ can be joined together to form a ring with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl and —S(O)nC₁₋₄alkyl.

6. A compound according to claim 5 wherein
R₂ is selected from the group consisting of
(1) phenyl,
(2) —C
(3) —C(O)NR₉R₁₀,
wherein R₉ and R₁₀ are each independently selected from the group consisting of
(a) aryl,
(b) HET₄,
(c) —C₃₋₆cycloalkyl, optionally substituted with 1 to 4 methyl groups,
(d) —C₁₋₄alkyl, optionally mono or di-substituted with hydroxyl, HET₅, or C₃₋₆cycloalkyl,
or
R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
wherein R₂ choices (1), (2) and (3), are each optionally mono or di-substituted with substituents independently selected from the group consisting of:
(a) halo,
(b) —CN,
(e) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino,
(f) —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
wherein R₁₁, R₁₂, R₁₃, R₁₄, R₁₅, R₁₆, R₁₇, R₁₈, are each independently selected from H and C₁₋₄alkyl, optionally substituted with hydroxyl.

7. A compound according to claim 6 wherein
R₂ is selected from the group consisting of:
(1) phenyl,
(2) —C₁₋₆alkyl,
(3) —C(O)NR₉R₁₀,
wherein R₉ and R₁₀ are each independently selected from the group consisting of
(a) aryl,
(b) HET₄,
(c) —C₃₋₆cycloalkyl, optionally substituted with 1 to 4 methyl groups,
(d) —C₁₋₄alkyl, optionally mono or di-substituted with hydroxyl, HET₅, or C₃₋₆cycloalkyl,
or
R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
wherein R₂ choices (1), (2) and (3), are each optionally mono or di-substituted with substituents independently selected from the group consisting of:
(a) halo,
(b) mono, di or tri-halo C₁₋₄ alkyl,
(c) —OC₁₋₄ alkyl, optionally substituted with hydroxyl, halo or amino,
(d) —C₁₋₄alkyl optionally substituted with one or two substituents selected from hydroxyl, CN, —CHF₂, —CF₃, —NH₂, and —OCH₃,
(e) —C(O)—O-aryl,
(f) —C(O)—NR₁₃R₁₄,
(g) —NR₁₇R₁₈, and
(h) hydroxyl,
wherein R₁₃, R₁₄, R₁₇, R₁₈, are each independently selected from H and C₁₋₄alkyl, optionally substituted with hydroxyl.

8. A compound according to claim 1 wherein
R₃ is selected from the group consisting of:
(1) aryl, and
(2) HET₇,
wherein choices (1) and (2) are each optionally mono or di-substituted with substituents independently selected from the group consisting of:
(a) halo, and
(b) methyl.

9. A compound according to claim 8 wherein
R₃ is an optionally substituted:
(1) phenyl,
(2) pyridyl,
(3) pyridazinyl, and
(4) pyrimidyl.

10. A compound according to claim 1 wherein
R₄ and R₅ are each hydrogen.

11. A compound according to claim 1 wherein
R₇ is selected from the group consisting of:
(1) hydrogen,
(2) halogen, and
(3) HET₈,
wherein choice (3) is optionally mono or di-substituted with substituents selected from hydroxyl, C₃₋₆cycloalkyl, —C(O)—NH₂, phenyl and HET₉.

12. A compound according to claim 1 of the formula 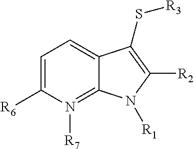
or a pharmaceutically acceptable salt thereof wherein
n is 0, 1 or 2;
R₁ is selected from the group consisting of:
(1) hydrogen, and
(2) C₁₋₄alkyl,
wherein choice (2), is optionally mono or di-substituted with substituents selected from hydroxyl, halo, CF₃ and OCH₃;
R₂ is selected from the group consisting of
(1) phenyl,
(2) —C₁₋₆alkyl, and
(3) —C(O)NR₉R₁₀,
wherein R₉ and R₁₀ are each independently selected from the group consisting of
(a) aryl,
(b) HET₄,
(c) —C₃₋₆cycloalkyl, optionally substituted with 1 to 4 methyl groups,
(d) —C₁₋₄alkyl, optionally mono or di-substituted with hydroxyl, HET₅, or C₃₋₆cycloalkyl,
or
R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
wherein R₂ choices (1), (2) and (3), are each optionally mono or di-substituted with substituents independently selected from the group consisting of:
(a) halo,
(b) —CN,
(c) mono, di or tri-halo C₁₋₄ alkyl,
(d) mono, di or tri-halo OC₁₋₄ alkyl,
(e) —OC₁₋₄ alkyl, optionally substituted with hydroxyl, halo or amino, —C₁₋₄alkyl optionally substituted with one or two substituents selected from hydroxyl, CN, —CHF₂, —CF₃, —NH₂, and —OCH₃,
(g) —C₂₋₆alkenyl optionally substituted with one or two substituents selected from hydroxyl, CN, —CHF₂, —CF₃, —NH₂, and —OCH₃,
(h) —C₃₋₆cycloalkyl optionally substituted with hydroxy, halo or CN,
(i) —S(O)ₙC₁₋₄alkyl,
(j) —S(O)ₙNR₁₁R₁₂,
(k) —C(O)—OH,
(l) —C(O)—OC₁₋₄alkyl, optionally substituted with halo, hydroxy, phenyl or methoxy, wherein the phenyl is optionally substituted with halo, hydroxy, phenyl or methoxy,
(m) —C(O)—O-aryl,
(n) —C(O)—NR₁₃R₁₄,
(o) —C(O)—C₁₋₄alkyl optionally mono, di or tri substituted with halo,
(p) —C₁₋₄alkyl-C(O)—O—C₁₋₄alkyl, whereas the CH₂ may be optionally substituted with C₁₋₄alkyl or hydroxyl,
(q) —CH₂—C(O)NR₁₅R₁₆, whereas the CH₂ may be optionally substituted with C₁₋₄alkyl or hydroxy,
(r) —NR₁₇R₁₈,
(s) hydroxyl, and
(t) oxo,
wherein R₆, R₇, R₈, R₉, R₁₀, R₁₁, R₁₂, R₁₃, R₁₄, R₁₅, R₁₆, R₁₇, R₁₈, are each independently selected from H and C₁₋₄alkyl, optionally substituted with hydroxyl;
R₃ is selected from the group consisting of:
(1) aryl, and
(2) HET₇,
wherein choices (1) and (2) are each optionally mono or di-substituted with substituents independently selected from the group consisting of:
(a) halo, and
(b) methyl;
R₆ is selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₇cycloalkyl;
wherein choice (7), and the aryl or HET₅ of choices (3), (4), (5) and (6) are optionally mono or di-substituted with substituents selected from hydroxyl, halo, CF₃ and OCH₃; and
R₇ is selected from the group consisting of:
(1) hydrogen,
(2) halogen, and
(3) HET₈,
wherein choice (3) is optionally mono or di-substituted with substituents selected from hydroxyl, C₃₋₆cycloalkyl, —C(O)—NH₂, phenyl and HET₉.

13. A compound according to claim 12 of the formula: 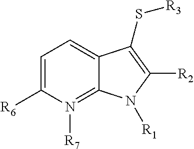
or a pharmaceutically acceptable salt thereof wherein:
is 0, 1 or 2;
R₁ is selected from the group consisting of
(1) hydrogen, and
(2) C₁₋₄alkyl,
wherein choice (2), is optionally mono or di-substituted with substituents selected from hydroxyl, halo, CF₃ and OCH₃;
R₂ is selected from the group consisting of:
(1) phenyl,
(2) —C₁₋₆alkyl,
(3) —C(O)NR₉R₁₀,
wherein R₉ and R₁₀ are each independently selected from the group consisting of
(a) aryl,
(b) HET₄,
(c) —C₃₋₆cycloalkyl, optionally substituted with 1 to 4 methyl groups,
(d) —C₁₋₄alkyl, optionally mono or di-substituted with hydroxyl, HET₅, or C₃₋₆cycloalkyl,
or
R₉ and R₁₀ are joined together to form a ring with the atoms to which they are attached there is formed a heterocyclic ring of 5 or 6 atoms, said ring containing 1, or 2 heteroatoms selected from N, O and S, said ring being optionally mono or di-substituted with substituents independently selected from hydroxyl, —C(O)—C₁₋₄alkyl, and C(O)—NRaRb, wherein Ra and Rb are each independently selected from hydrogen and methyl,
wherein R₂ choices (1), (2) and (3), are each optionally mono or di-substituted with substituents independently selected from the group consisting of
(a) halo,
(b) mono, di or tri-halo C₁₋₄ alkyl,
(c) —OC₁₋₄ alkyl, optionally substituted with hydroxyl, halo or amino,
(d) —C₁₋₄alkyl optionally substituted with one or two substituents selected from hydroxyl, CN, —CHF₂, —CF₃, —NH₂, and —OCH₃,
(e) —C(O)—O-aryl,
(f) —C(O)—NR₁₃R₁₄,
(g) —NR₁₇R₁₈, and
(h) hydroxyl,
wherein R₁₃, R₁₄, R₁₇, R₁₈, are each independently selected from H and C₁₋₄ alkyl, optionally substituted with hydroxyl;
R₃ is selected from
(1) phenyl,
(2) pyridyl,
(3) pyridazinyl, and
(4) pyrimidyl,
wherein R₃ is optionally mono or di substituted with substituents selected from the group consisting of halo and methyl;
R₆ is selected from the group consisting of:
(1) hydrogen,
(2) halogen,
(3) aryl,
(4) HET₅,
(5) (CH₂)-aryl,
(6) (CH₂)-HET₅,
(7) —C₁₋₆alkyl, and
(8) —C₃₋₇cycloalkyl;
wherein choice (7), and the aryl or HET₅ of choices (3), (4), (5) and (6) are optionally mono or di-substituted with substituents selected from hydroxyl, halo, CF₃ and OCH₃; and
R₇ is selected from the group consisting of:
(1) hydrogen,
(2) halogen, and
(3) HET₈,
wherein choice (3) is optionally mono or di-substituted with substituents selected from hydroxyl, C₃₋₆cycloalkyl, —C(O)—NH₂, phenyl and HET₉.

14. A compound according to claim 1 selected from the group consisting of
3-[(4-chlorophenyl)sulfanyl]-2-[4-(methylsulfanyl)phenyl]-1H-
pyrrolo[2,3-b]pyridine,
3-[(4-chlorophenyl)sulfanyl]-2-[4-(methylsulfanyl)phenyl]-1H-
pyrrolo[2,3-b]pyridine,
1-(4-{3-[(4-chlorophenyl)sulfanyl]-1H-pyrrolo[2,3-b]pyridin-2-
yl}phenyl)-2,2,2-trifluoroethanol,
2-(4-{3-[(5-chloropyridin-2-yl)sulfanyl]-1H-pyrrolo[2,3-b]pyridin-2-
yl}phenyl)propan-2-ol,
3-[(5-chloropyridin-2-yl)sulfanyl]-2-[4-(methylsulfanyl)phenyl]-1H-
pyrrolo[2,3-b]pyridine,
3-[(5-chloropyridin-2-yl)sulfanyl]-2-[6-(methylsulfinyl)pyridin-3-yl]-
1H-pyrrolo[2,3-b]pyridine,
3-[(4-chlorophenyl)sulfanyl]-2-[6-(methylsulfanyl)pyridin-3-yl]-1H-
pyrrolo[2,3-b]pyridine,
3-[(4-chlorophenyl)sulfanyl]-2-[4-(propan-2-yloxy)phenyl]-1H-
pyrrolo[2,3-b]pyridine,
3-[(4-chlorophenyl)sulfanyl]-2-[2-(methoxymethyl)phenyl]-1H-
pyrrolo[2,3-b]pyridine,
3-[(5-chloropyridin-2-yl)sulfanyl]-2-[4-(methylsulfanyl)phenyl]-1H-
pyrrolo[2,3-b]pyridine,
N-(4-{3-[(4-chlorophenyl)sulfanyl]-1H-pyrrolo[2,3-b]pyridin-2-
yl}phenyl)acetamide,
3-[(5-chloropyridin-2-yl)sulfanyl]-2-[4-(morpholin-4-ylsulfonyl)phenyl]-
1H-pyrrolo[2,3-b]pyridine,
N-tert-butyl-4-{3-[(4-chlorophenyl)sulfanyl]-1H-pyrrolo[2,3-b]pyridin-
2-yl}benzenesulfonamide,
4-{3-[(4-chlorophenyl)sulfanyl]-1H-pyrrolo[2,3-b]pyridin-2-yl}phenol,
3-[(5-chloropyridin-2-yl)sulfanyl]-2-(6-methoxypyridin-3-yl)-1H-
pyrrolo[2,3-b]pyridine,
1-(4-{3-[(5-chloropyridin-2-yl)sulfanyl]-1H-pyrrolo[2,3-b]pyridin-2-
yl}phenyl)-2,2,2-trifluoroethanol,
methyl 4-{3-[(4-chlorophenyl)sulfanyl]-1H-pyrrolo[2,3-b]pyridin-2-
yl}benzoate,
4-{3-[(4-chlorophenyl)sulfanyl]-1H-pyrrolo[2,3-b]pyridin-2-yl}aniline,
4-{3-[(5-chloropyridin-2-yl)sulfanyl]-1H-pyrrolo[2,3-b]pyridin-2-
yl}cyclohex-3-en-1-ol,
(4-{3-[(4-chlorophenyl)sulfanyl]-1H-pyrrolo[2,3-b]pyridin-2-
yl}phenyl)methanol,
1-(4-{3-[(4-chlorophenyl)sulfanyl]-1H-pyrrolo[2,3-b]pyridin-2-
yl}phenyl)methanamine,
2-(4-{3-[(4-chlorophenyl)sulfanyl]-1H-pyrrolo[2,3-b]pyridin-2-
yl}phenyl)propan-2-ol,
5-{3-[(4-chlorophenyl)sulfanyl]-1H-pyrrolo[2,3-b]pyridin-2-yl}-2,3-
dihydro-1H-isoindol-1-one,
5-{3-[(5-chloropyridin-2-yl)sulfanyl]-1H-pyrrolo[2,3-b]pyridin-2-yl}-
1,3-dihydro-2H-indol-2-one,
5-{3-[(5-chloropyridin-2-yl)sulfanyl]-1H-pyrrolo[2,3-b]pyridin-2-yl}-
2,3-dihydro-1H-isoindol-1-one,
3-[(5-chloropyridin-2-yl)sulfanyl]-2-[2-(methylsulfinyl)pyrimidin-5-yl]-
1H-pyrrolo[2,3-b]pyridine,
3-[(4-chlorophenyl)sulfanyl]-2-[6-(methylsulfinyl)pyridin-3-yl]-1H-
pyrrolo[2,3-b]pyridine,
3-[(4-chlorophenyl)sulfanyl]-2-(2,3-dihydro-1,4-benzodioxin-6-yl)-1H-
pyrrolo[2,3-b]pyridine
3-[(4-chlorophenyl)sulfanyl]-2-(3-methoxyphenyl)-1H-pyrrolo[2,3-
b]pyridine
3-[(4-chlorophenyl)sulfanyl]-2-(4-methoxyphenyl)-1H-pyrrolo[2,3-
b]pyridine
3-[(4-chlorophenyl)sulfanyl]-2-(3,4-dimethoxyphenyl)-1H-pyrrolo[2,3-
b]pyridine
3-[(4-chlorophenyl)sulfanyl]-2-(1H-indol-4-yl)-1H-pyrrolo[2,3-
b]pyridine
3-[(4-chlorophenyl)sulfanyl]-2-(1-methyl-1H-indol-5-yl)-1H-
pyrrolo[2,3-b]pyridine
1-(4-{3-[(4-chlorophenyl)sulfanyl]-1H-pyrrolo[2,3-b]pyridin-2-
yl}phenyl)ethanol,
3-[(5-chloropyridin-2-yl)sulfanyl]-2-(2,3-dihydro-1,4-benzodioxin-6-yl)-
1H-pyrrolo[2,3-b]pyridine,
3-[(4-chlorophenyl)sulfanyl]-2-(2,4-dimethoxypyrimidin-5-yl)-1H-
pyrrolo[2,3-b]pyridine-methane,
3-[(4-chlorophenyl)sulfanyl]-2-(cyclopent-1-en-1-yl)-1H-pyrrolo[2,3-
b]pyridine
4-{3-[(4-chlorophenyl)sulfanyl]-1H-pyrrolo[2,3-b]pyridin-2-
yl}cyclohex-3-ene-1-carbonitrile,
3-[(4-chlorophenyl)sulfanyl]-2-(5,6-dihydro-2H-pyran-3-yl)-1H-
pyrrolo[2,3-b]pyridine,
3-[(4-chlorophenyl)sulfanyl]-2-(2,6-dimethoxypyridin-3-yl)-1H-
pyrrolo[2,3-b]pyridine,
3-[(4-chlorophenyl)sulfanyl]-2-(3,5-dimethyl-1H-pyrazol-4-yl)-1H-
pyrrolo[2,3-b]pyridine,
3-[(4-chlorophenyl)sulfanyl]-2-(1-methyl-1H-pyrazol-4-yl)-1H-
pyrrolo[2,3-b]pyridine,
3-[(4-chlorophenyl)sulfanyl]-1H,1′H-2,4′-bipyrrolo[2,3-b]pyridine,
3-[(4-chlorophenyl)sulfanyl]-2-(thiophen-3-yl)-1H-pyrrolo[2,3-
b]pyridine,
1-(5-{3-[(4-chlorophenyl)sulfanyl]-1H-pyrrolo[2,3-b]pyridin-2-
yl}thiophen-2-yl)ethanone,
3-[(4-chlorophenyl)sulfanyl]-2-(3,5-dimethylisoxazol-4-yl)-1H-
pyrrolo[2,3-b]pyridine,
3-[(4-chlorophenyl)sulfanyl]-2-(5,6-dihydro-4H-pyrrolo[1,2-b]pyrazol-
3-yl)-1H-pyrrolo[2,3-b]pyridine,
4-{3-[(4-chlorophenyl)sulfanyl]-1H-pyrrolo[2,3-b]pyridin-2-
yl}thiophene-3-carbonitrile,
3-[(4-chlorophenyl)sulfanyl]-2-(thiophen-2-yl)-1H-pyrrolo[2,3-
b]pyridine,
(3E)-4-{3-[(4-chlorophenyl)sulfanyl]-1H-pyrrolo[2,3-b]pyridin-2-yl}-2-
methylbut-3-en-2-ol,
3-[(4-chlorophenyl)sulfanyl]-2-(4,5,6,7-tetrahydropyrazolo[1,5-
a]pyridin-3-yl)-1H-pyrrolo[2,3-b]pyridine,
4-{3-[(4-chlorophenyl)sulfanyl]-1H-pyrrolo[2,3-b]pyridin-2-
yl}cyclohex-3-en-1-ol,
3-[(4-chlorophenyl)sulfanyl]-2-(6-cyclopropylpyridin-3-yl)-1H-
pyrrolo[2,3-b]pyridine,
(4E)-5-{3-[(4-chlorophenyl)sulfanyl]-1H-pyrrolo[2,3-6]pyridin-2-
yl}pent-4-en-2-ol,
3-[(4-chlorophenyl)sulfanyl]-2-{(E)-2-[4-
(trifluoromethyl)phenyl]ethenyl}-1H-pyrrolo[2,3-b]pyridine,
3-[(4-chlorophenyl)sulfanyl]-2-(cyclohex-1-en-1-yl)-1H-pyrrolo[2,3-
b]pyridine,
3-[(4-chlorophenyl)sulfanyl]-2-(5,6-dimethoxypyridin-3-yl)-1H-
pyrrolo[2,3-b]pyridine,
5-{3-[(4-chlorophenyl)sulfanyl]-1H-pyrrolo[2,3-b]pyridin-2-yl}-2-
methyl-2H-indazole,
3-[(4-chlorophenyl)sulfanyl]-2-(6-methoxypyridin-3-yl)-1H-pyrrolo[2,3-
b]pyridine,
5-{3-[(5-chloropyridin-2-yl)sulfanyl]-1H-pyrrolo[2,3-b]pyridin-2-yl}-2-
methyl-2H-indazole,
3-[(4-chlorophenyl)sulfanyl]-2-[4-(difluoromethoxy)phenyl]-1H-
pyrrolo[2,3-b]pyridine,
4-{3-[(4-chlorophenyl)sulfanyl]-1H-pyrrolo[2,3-b]pyridin-2-
yl}cyclohex-3-en-1-amine,
4-{3-[(4-chlorophenyl)sulfanyl]-1H-pyrrolo[2,3-b]pyridin-2-
yl}cyclohex-3-ene-1-carboxylic acid,
4-{3-[(4-chlorophenyl)sulfanyl]-1H-pyrrolo[2,3-b]pyridin-2-yl}-N-
ethylcyclohex-3-ene-1-carboxamide,
4-{3-[(4-chlorophenyl)sulfanyl]-1H-pyrrolo[2,3-b]pyridin-2-yl}-N-(2-
hydroxyethyl)cyclohex-3-ene-1-carboxamide,
(4-{3-[(4-chlorophenyl)sulfanyl]-1H-pyrrolo[2,3-b]pyridin-2-
yl}cyclohex-3-en-1-yl)methanol,
(cis-4-{3-[(4-chlorophenyl)sulfanyl]-1H-pyrrolo[2,3-b]pyridin-2-
yl}cyclohexyl)methanol,
(trans-4-{3-[(4-chlorophenyl)sulfanyl]-1H-pyrrolo[2,3-b]pyridin-2-
yl}cyclohexyl)methanol,
3-[(4-chlorophenyl)sulfanyl]-N-(4-methoxyphenyl)-1H-pyrrolo[2,3-
b]pyridine-2-carboxamide,
3-[(4-chlorophenyl)sulfanyl]-N-(6-methoxypyridin-3-yl)-1H-
pyrrolo[2,3-b]pyridine-2-carboxamide,
3-[(4-chlorophenyl)sulfanyl]-N-(4,4-dimethylcyclohexyl)-1H-
pyrrolo[2,3-b]pyridine-2-carboxamide,
3-[(4-chlorophenyl)sulfanyl]-N-(2-cyclohexyl-2-hydroxyethyl)-1H-
pyrrolo[2,3-b]pyridine-2-carboxamide,
3-[(4-chlorophenyl)sulfanyl]-N-(3,4-dihydroxybenzyl)-1H-pyrrolo[2,3-
b]pyridine-2-carboxamide,
{3-[(4-chlorophenyl)sulfanyl]-1H-pyrrolo[2,3-b]pyridin-2-yl}(2,3-
dihydro-1H-pyrrolo[2,3-c]pyridin-1-yl)methanone,
3-[(4-chlorophenyl)sulfanyl]-N-[1-(2,3-dihydro-1,4-benzodioxin-2-
yl)ethyl]-N-methyl-1H-pyrrolo[2,3-b]pyridine-2-carboxamide,
3-[(4-chlorophenyl)sulfanyl]-N-(2,3-dihydro-1,4-benzodioxin-6-yl)-1H-
pyrrolo[2,3-b]pyridine-2-carboxamide,
N-(1,3-benzodioxol-5-yl)-3-[(4-chlorophenyl)sulfanyl]-1H-pyrrolo[2,3-
b]pyridine-2-carboxamide,
3-[(4-chlorophenyl)sulfanyl]-N-(1H-indazol-5-yl)-1H-pyrrolo[2,3-
b]pyridine-2-carboxamide,
3-[(4-chlorophenyl)sulfanyl]-N-cyclohexyl-1H-pyrrolo[2,3-b]pyridine-2-
carboxamide,
3-[(4-chlorophenyl)sulfanyl]-N-(2-hydroxyethyl)-1H-pyrrolo[2,3-
b]pyridine-2-carboxamide,
3-[(4-chlorophenyl)sulfanyl]-N-(3-hydroxypropyl)-1H-pyrrolo[2,3-
b]pyridine-2-carboxamide,
3[(4-chlorophenyl)sulfanyl]-N-[(2R)-1-hydroxypropan-2-yl]-1H-
pyrrolo[2,3-b]pyridine-2-carboxamide,
3-[(4-chlorophenyl)sulfanyl]-N-(4-hydroxybutyl)-1H-pyrrolo[2,3-
b]pyridine-2-carboxamide,
3-[(4-chlorophenyl)sulfanyl]-N-{[4-(hydroxymethyl)tetrahydro-2H-
pyran-4-yl]methyl}-1H-pyrrolo[2,3-b]pyridine-2-carboxamide,
5-{3-[(4-chlorophenyl)sulfanyl]-7-methyl-7H-pyrrolo[2,3-b]pyridin-2-
yl}-1H-indazole,
4-{3-[(4-chlorophenyl)sulfanyl]-7-methyl-7H-pyrrolo[2,3-b]pyridin-2-
yl}cyclohex-3-en-1-ol,
2-(1,3-benzodioxol-5-yl)-3-[(4-chlorophenyl)sulfanyl]-7-methyl-7H-
pyrrolo[2,3-b]pyridine,
trans-4-{3-[(4-chlorophenyl)sulfanyl]-7-methyl-7H-pyrrolo[2,3-
b]pyridin-2-yl}cyclohexanol,
3-[(4-chlorophenyl)sulfanyl]-2-(5,6-dimethoxypyridin-3-yl)-7-methyl-
7H-pyrrolo[2,3-b]pyridine,
5-{3-[(5-chloropyridin-2-yl)sulfanyl]-7-methyl-7H-pyrrolo[2,3-
b]pyridin-2-yl}-1H-indazole,
1-(4-{3-[(4-chlorophenyl)sulfanyl]-7-methyl-7H-pyrrolo[2,3-b]pyridin-
2-yl}phenyl)-2,2,2-trifluoroethanol,
2-(1,3-benzodioxol-5-yl)-3-[(5-chloropyridin-2-yl)sulfanyl]-7-methyl-
7H-pyrrolo[2,3-b]pyridine,
2-{2-(1,3-benzodioxol-5-yl)-3-[(4-chlorophenyl)sulfanyl]-7H-
pyrrolo[2,3-b]pyridin-7-yl}ethanol,
2-(1,3-benzodioxol-5-yl)-3-[(4-chlorophenyl)sulfanyl]-7-ethyl-7H-
pyrrolo[2,3-b]pyridine,
1-(4-{3-[(5-chloropyridin-2-yl)sulfanyl]-7-methyl-7H-pyrrolo[2,3-
b]pyridin-2-yl}phenyl)-2,2,2-trifluoroethanol,
2-(1,3-benzodioxol-5-yl)-3-[(4-chlorophenyl)sulfanyl]-7-
(cyclopropylmethyl)-7H-pyrrolo[2,3-b]pyridine,
trans-4-{3-[(5-chloropyridin-2-yl)sulfanyl]-7-methyl-7H-pyrrolo[2,3-
b]pyridin-2-yl}cyclohexanol,
2-{3-[(5-chloropyridin-2-yl)sulfanyl]-2-(2,3-dihydro-1,4-benzodioxin-6-
yl)-7H-pyrrolo[2,3-b]pyridin-7-yl}ethanol,
3-[(4-chlorophenyl)sulfanyl]-N-(2,3-dihydro-1,4-benzodioxin-6-yl)-7-
methyl-7H-pyrrolo[2,3-b]pyridine-2-carboxamide,
4-{3-[(5-chloropyridin-2-yl)sulfanyl]-7-methyl-7H-pyrrolo[2,3-
b]pyridin-2-yl}cyclohex-3-en-1-ol,
2-{2-(1,3-benzodioxol-5-yl)-3-[(5-chloropyridin-2-yl)sulfanyl]-7H-
pyrrolo[2,3-b]pyridin-7-yl}ethanol,
3-{2-(1,3-benzodioxol-5-yl)-3-[(4-chlorophenyl)sulfanyl]-7H-
pyrrolo[2,3-b]pyridin-7-yl}propanamide,
2-(1,3-benzodioxol-5-yl)-3-[(4-chlorophenyl)sulfanyl]-7-[2-(1H-pyrrol-
1-yl)ethyl]-7H-pyrrolo[2,3-b]pyridine,
cis-4-{3-[(5-chloropyridin-2-yl)sulfanyl]-7-methyl-7H-pyrrolo[2,3-
b]pyridin-2-yl}cyclohexanol,
3-[(4-chlorophenyl)sulfanyl]-N-(1H-indazol-5-yl)-7-methyl-7H-
pyrrolo[2,3-b]pyridine-2-carboxamide,
1-(4-{3-[(4-chlorophenyl)sulfanyl]-7-methyl-7H-pyrrolo[2,3-b]pyridin-
2-yl}phenyl)-2,2-difluoroethanol,
3-[(4-chlorophenyl)sulfanyl]-N-(4-hydroxycyclohexyl)-7-methyl-7H-
pyrrolo[2,3-b]pyridine-2-carboxamide,
(3S)-4-{3-[(4-chlorophenyl)sulfanyl]-7-methyl-7H-pyrrolo[2,3-
b]pyridin-2-yl}-2,3-dimethylbutan-2-ol,
5-{3-[(4-chlorophenyl)sulfanyl]-7-methyl-7H-pyrrolo[2,3-b]pyridin-2-
yl}-2,3-dihydro-1H-isoindol-1-one,
5-{3-[(4-chlorophenyl)sulfanyl]-7-methyl-7H-pyrrolo[2,3-b]pyridin-2-
yl}-2-methyl-2H-indazole,
5-{3-[(5-chloropyridin-2-yl)sulfanyl]-7-methyl-7H-pyrrolo[2,3-
b]pyridin-2-yl}-2-methyl-2H-indazole,
5-{3-[(5-chloropyridin-2-yl)sulfanyl]-7-methyl-7H-pyrrolo[2,3-
b]pyridin-2-yl}-2,3-dihydro-1H-isoindol-1-one,
methyl 4-{3-[(4-chlorophenyl)sulfanyl]-7-methyl-7H-pyrrolo[2,3-
b]pyridin-2-yl}piperidine-1-carboxylate,
1-(4-{3-[(4-chlorophenyl)sulfanyl]-1-methyl-1H-pyrrolo[2,3-b]pyridin-
2-yl}phenyl)-2,2,2-trifluoroethanol,
trans-4-{3-[(4-chlorophenyl)sulfanyl]-1-methyl-1H-pyrrolo[2,3-
b]pyridin-2-yl}cyclohexanol,
2-{2-(1,3-benzodioxol-5-yl)-3-[(4-chlorophenyl)sulfanyl]-1H-
pyrrolo[2,3-b]pyridin-1-yl}ethanol,
ethyl 3-{3-[(4-chlorophenyl)sulfanyl]-1H-pyrrolo[2,3-b]pyridin-2-
yl}propanoate,
3-[(4-chlorophenyl)sulfanyl]-2-(cyclohexylmethyl)-1H-pyrrolo[2,3-
b]pyridine,
methyl (2S)-3-{3-[(4-chlorophenyl)sulfanyl]-1H-pyrrolo[2,3-b]pyridin-
2-yl}-2-methylpropanoate,
4-{3-[(4-chlorophenyl)sulfanyl]-1H-pyrrolo[2,3-b]pyridin-2-
yl}butanenitrile,
3-[(4-chlorophenyl)sulfanyl]-2-(5-methylpyridin-2-yl)-1H-pyrrolo[2,3-
b]pyridine,
2-{3-[(4-chlorophenyl)sulfanyl]-1H-pyrrolo[2,3-b]pyridin-2-
yl}benzonitrile,
3-[(4-chlorophenyl)sulfanyl]-2-(pyridin-2-yl)-1H-pyrrolo[2,3b]pyridine,
ethyl 4-{3-[(4-chlorophenyl)sulfanyl]-1H-pyrrolo[2,3-b]pyridin-2-
yl}butanoate
1-(4-{3-[(4-chlorophenyl)sulfanyl]-1H-pyrrolo[2,3-b]pyridin-2-
yl}piperidin-1-yl)-2-methylpropan-2-ol,
4-{3-[(4-chlorophenyl)sulfanyl]-1H-pyrrolo[2,3-b]pyridin-2-yl}butan-1-
ol,
(2S)-3-{3-[(4-chlorophenyl)sulfanyl]-1H-pyrrolo[2,3-b]pyridin-2-yl}-2-
methylpropan-1-ol,
(3S)-4-{3-[(4-chlorophenyl)sulfanyl]-1H-pyrrolo[2,3-b]pyridin-2-yl}-
2,3-dimethylbutan-2-ol,
N-({3-[(4-chlorophenyl)sulfanyl]-1H-pyrrolo[2,3-b]pyridin-2-
yl}methyl)tetrahydro-2H-pyran-4-amine,
1-({3-[(4-chlorophenyl)sulfanyl]-1H-pyrrolo[2,3-b]pyridin-2-
yl}methyl)piperidin-4-ol,
1,3-benzodioxol-5-yl{3-[(4-chlorophenyl)sulfanyl]-1H-pyrrolo[2,3-
b]pyridin-2-yl}methanol,
{3-[(4-chlorophenyl)sulfanyl]-1H-pyrrolo[2,3-b]pyridin-2-yl}(4-
methoxyphenyl)methanol,
1-{3-[(4-chlorophenyl)sulfanyl]-1H-pyrrolo[2,3-b]pyridin-2-yl}-2-
methylpropan-1-ol, and
N-({3-[(4-chlorophenyl)sulfanyl]-1H-pyrrolo[2,3-b]pyridin-2-yl}methyl)-
2,2,6,6-tetramethyltetrahydro-2H-pyran-4-amine,
or a pharmaceutically acceptable salt thereof.

15. A pharmaceutical composition which comprises an inert carrier and a compound of claim 1 or a pharmaceutically acceptable salt thereof.

16. A method of treating a FAAH mediated disease in a patient in need of such treatment comprising: administration to a patient in need of such treatment of a therapeutically effective amount of a compound of formula I, according to claim 1 and a pharmaceutically acceptable carrier.

17. A method according to claim 14, wherein the disease is selected from osteoarthritis, rheumatoid arthritis, diabetic neuropathy, postherpetic neuralgia, pain, fibromyalgia, pain, migraine, sleep disorder, Alzheimer Disease, and Parkinson's Disease.

18. Use of a compound according to claim 1 or a pharmaceutically acceptable salt thereof for the manufacture of a medicament for the treatment of a physiological disorder associated with an excess of FAAH in a mammal.


SEQUENCE LISTING (3 sequences)

SEQ ID NO: 1 - DNA, 36 bp, Unknown
  Primer
caaggtaccgccaccatggtgctgagcgaagtgtgg

SEQ ID NO: 2 - DNA, 30 bp, Unknown
  Primer
ccggaattctcaagatggccgcttttcagg

SEQ ID NO: 3 - DNA, 30 bp, Unknown
  Primer
ccggaattctcacgatggctgcttttgagg